(19) United States
(12) Patent Application Publication
(10) Pub. No.: US 2025/0002905 A1
(43) Pub. Date: Jan. 2, 2025

(54) MODIFIED CELLS AND METHODS OF USE THEREOF

(71) Applicant: New York Stem Cell Foundation, Inc., New York, NY (US)

(72) Inventors: Frederick J. Monsma, Jr., Summit, NJ (US); Paula Alonso-Guallart, Brooklyn, NY (US); Josephine Wesely, New York, NY (US)

(21) Appl. No.: 18/701,023

(22) PCT Filed: Oct. 14, 2022
(86) PCT No.: PCT/US2022/046790

§ 371 Date: Apr. 12, 2024

Related U.S. Application Data
(60) Provisional application No. 63/255,830, filed on Oct. 14, 2021.

(51) Int. Cl. C12N 15/11; A61K 35/545; A61P 3/10; C12N 5/0735; C12N 9/22; C12N 15/86

(52) U.S. Cl. CPC C12N 15/11; A61K 35/545; A61P 3/10; C12N 5/0606; C12N 9/22; C12N 15/86; C12N 2310/20; C12N 2510/00; C12N 2740/15043

(57) ABSTRACT

The disclosure relates to mammalian cells (e.g., stem cells) that are modified with respect to expression of one or a combination of the following genes: SIGLEC14, MUC15, GPC4, and TEX10. Related methods of making and using such modified mammalian cells are also disclosed.

### CROSS-REFERENCE TO RELATED APPLICATIONS

This application claims the benefit of and priority to U.S. Provisional Patent Application No. 63/255,830, filed Oct. 14, 2021, the disclosure of which is hereby incorporated by reference in its entirety for all purposes.

### FIELD OF THE INVENTION

The invention relates generally to mammalian cells, including stem cells, which have been modified to promote evasion of an immune response, and methods of making and using such modified cells.

### BACKGROUND

Regenerative medicine in the form of cell transplantation is one of the most promising therapeutic approaches for the treatment of intractable medical conditions such as diabetes, heart disease, and neurodegenerative diseases. However, a major hurdle toward implementing cell transplantation in the clinic is immune rejection of donor cells with a disparate genetic background. While it is possible to address immune rejection, in part, by administering immunosuppressant drugs, these typically entail severe adverse side effects.

Organ transplantation provides an opportunity to treat people with certain diseases and can allow an organ recipient to live a full life. For example, in the case of end-stage liver, lung, and heart disease, transplantation is generally the only available therapeutic option. There have been improvements in immunosuppressive drugs and ancillary care that have led to increased short-term patient and graft survival rates. However, this success is hampered by several problems, such as poor long-term graft survival rates, the need for continual immunosuppressive medication and the discrepancy between supply and demand of organs.

Allotransplantations have been developed to provide a supply of donor tissue. However, a major challenge limits this approach: allogeneic transplants fail unless the recipient's immune system is downregulated. The current clinical standard is the use of systemic immunosuppressive medications, which reduce the efficacy of the graft and substantially increase the risk of infections.

Hence, there remains unmet needs in understanding immune surveillance and the ability to genetically modify cells, such as stem cells, to generate cells that evade immune rejection.

### SUMMARY

The present disclosure is based, in part, upon the discovery that expression of one of the genes selected from SIGLEC14, MUC15, GPC4, and TEX10 by a mammalian cell can promote evasion of an immune response. Accordingly, the present disclosure provides new, modified mammalian cells that are modified with respect to expression of, e.g., one of the following genes or a combination of the following genes: SIGLEC14, MUC15, GPC4, TEX10. The disclosure also provides populations of modified mammalian cells, and tissues comprising and/or derived from such modified cells. Related methods of making and using such modified cells, cell populations, and tissues are also provided.

Accordingly, in one aspect, the present disclosure provides a modified mammalian cell in which a gene selected from the group consisting of SIGLEC14, MUC15, GPC4, TEX10, and combinations thereof, is overexpressed. In certain embodiments, at least two genes selected from the group consisting of SIGLEC14, MUC15, GPC4, and TEX10 are overexpressed. In certain embodiments, at least three genes selected from the group consisting of SIGLEC14, MUC15, GPC4, and TEX10 are overexpressed. In certain embodiments, SIGLEC14, MUC15, GPC4, and TEX10 are overexpressed.

In certain embodiments, the modified mammalian cell is further characterized by overexpression of: (a) one or more genes associated with inhibition of macrophage phagocytosis; (b) one or more genes associated with inhibition of complement activation and deposition; (c) one or more genes associated with inhibition of natural killer (NK) cell activation; or (d) any combination of (a), (b), and (c). In certain embodiments, the one or more genes associated with inhibition of macrophage phagocytosis comprises CD47. In certain embodiments, the one or more genes associated with complement activation and deposition comprise a gene selected from the group consisting of CD55, CD59, CD46, and combinations thereof. In certain embodiments, the one or more genes associated with inhibition of NK cell activation comprise a gene selected from the group consisting of HLA-G, HLA-E, and a combination thereof.

In certain embodiments, the modified mammalian cell is characterized by: (a) overexpression of one or more genes associated with inhibition of macrophage phagocytosis; (b) overexpression of one or more genes associated with inhibition of complement activation and deposition; and (c) overexpression of one or more genes associated with inhibition of NK cell activation. In certain embodiments, the modified mammalian cell is characterized by: (a) overexpression of CD47; (b) overexpression of CD55, CD59, and CD46; and (c) overexpression of HLA-G and of HLA-E.

In certain embodiments, the modified mammalian cell is characterized by: (a) overexpression of one or more genes associated with inhibition of macrophage phagocytosis; (b) overexpression of one or more genes associated with inhibition of complement activation and deposition; and (c) normal expression of HLA-G and of HLA-E. In certain embodiments, the modified mammalian cell is characterized by: (a) overexpression of CD47; (b) overexpression of CD55, CD59, and CD46; and (c) normal expression of HLA-G and of HLA-E.

In certain embodiments, the modified mammalian cell is characterized by: (a) normal expression of CD47; (b) overexpression of one or more genes associated with inhibition of complement activation and deposition; and (c) overexpression of one or more genes associated with inhibition of NK cell activation. In certain embodiments, the modified mammalian cell is characterized by: (a) normal expression of CD47; (b) overexpression of CD55, CD59, and CD46; and (c) overexpression of HLA-G and of HLA-E.

In certain embodiments, the modified mammalian cell is characterized by: (a) overexpression of one or more genes associated with inhibition of macrophage phagocytosis; (b) normal expression of CD55, CD59, and CD46; and (c) overexpression of one or more genes associated with inhibition of NK cell activation. In certain embodiments, the modified mammalian cell is characterized by: (a) overexpression of CD47; (b) normal expression of CD55, CD59, and CD46; and (c) overexpression of HLA-G and of HLA-E.

In certain embodiments, the modified mammalian cell is characterized by: (a) overexpression of one or more genes associated with inhibition of macrophage phagocytosis; (b) normal expression of CD55, CD59, and CD46; and (c) normal expression of HLA-G and of HLA-E. In certain embodiments, the modified mammalian cell is characterized by: (a) overexpression of CD47; (b) normal expression of CD55, CD59, and CD46; and (c) normal expression of HLA-G and of HLA-E.

In certain embodiments, the modified mammalian cell is characterized by: (a) normal expression of CD47; (b) overexpression of one or more genes associated with inhibition of complement activation and deposition; and (c) normal expression of HLA-G and of HLA-E. In certain embodiments, the modified mammalian cell is characterized by: (a) normal expression of CD47; (b) overexpression of CD55, CD59, and CD46; and (c) normal expression of HLA-G and of HLA-E.

In certain embodiments of any of the modified mammalian cells disclosed herein, each overexpressed gene is overexpressed by at least 1.5-fold relative to expression in a control cell.

In certain embodiments, the modified mammalian cell comprises one or more exogenous nucleic acid expression constructs, wherein each exogenous nucleic acid expression construct (1) encodes a gene and (2) comprises a promoter operably linked to the gene. In certain embodiments, the exogenous nucleic acid expression construct is a lentivirus construct. In certain embodiments, the exogenous nucleic acid expression construct is an adeno-associated virus (AAV) construct.

In certain embodiments, the modified mammalian cell comprises one or more exogenous gene activation systems, wherein each exogenous gene activation system is capable of driving expression of an endogenous gene. In certain embodiments, the gene activation system is a CRISPR activation system. In certain embodiments, said CRISPR activation system comprises: (a) an endonuclease-inactive Cas9 fused to a transcriptional activator; and (b) a guide RNA (gRNA) that is capable of hybridizing to an endogenous gene.

In certain embodiments, the modified mammalian cell is further characterized by abrogated expression of one or more genes associated with T-cell activation. In certain embodiments, the one or more genes associated with T-cell activation are selected from the group consisting of B2M, TAP1, CD74, CIITA, and combinations thereof.

In certain embodiments, the modified mammalian cell is further characterized by abrogated expression of one or more genes associated with NK cell activation. In certain embodiments, the one or more genes associated with NK cell activation comprise one or more NKG2D ligands. In certain embodiments, the one or more NKG2D ligands are selected from the group consisting of MICA, MICB, and a combination thereof.

In certain embodiments, the modified mammalian cell is characterized by abrogated expression of B2M, TAP1, CD74, CIITA, MICA, and MICB.

In certain embodiments, the modified mammalian cell is a human cell.

In certain embodiments, the modified mammalian cell is a stem cell. In certain embodiments, the stem cell is an induced pluripotent stem cell (iPSC). In certain embodiments, the stem cell is an embryonic stem cell (ESC).

In certain embodiments, the modified mammalian cell is capable of evading immune rejection as determined by an in vitro assay. In certain embodiments, the modified mammalian cell is capable of evading immune rejection as determined by an in vivo assay.

In another aspect, the present disclosure provides a cell or tissue that is differentiated from a modified mammalian cell of any of the foregoing embodiments.

In another aspect, the present disclosure provides a method comprising: administering a modified mammalian cell as disclosed herein to a mammalian subject, or administering a cell or tissue that is differentiated from a modified mammalian cell as disclosed herein to a mammalian subject. In certain embodiments, the mammalian subject is deficient in a cell type, and (1) the method comprises administering a modified mammalian stem cell as disclosed herein, and the modified mammalian stem cell is capable of developing into said cell type; or (2) the method comprises administering a cell or tissue that is differentiated from a modified mammalian cell as disclosed herein, and (i) the cell or tissue is capable of developing into said cell type or (ii) the cell or tissue comprises cells of said cell type.

In certain embodiments of the any of the foregoing methods, the mammalian subject has been diagnosed with or is at risk for diabetes.

In certain embodiments of any of the foregoing methods, the mammalian subject is a human.

### BRIEF DESCRIPTIONS OF THE DRAWINGS

FIG. 1 is a graphical illustration showing that teratoma growth was observed in immunocompetent mice transplanted with stem cells modified to express immune evasion genes. HLAI/IIKO hES cells were lentivirally transduced with the listed mouse immune evasion genes. Approximately 30% of cells were infected with any given lentivirus, leading to a relatively low frequency of cells expressing all 4 genes. These or control cells were transplanted in bulk into 5 WT C57B16/N mice and teratoma growth was measured over 8 weeks. Only cells receiving lentiviruses demonstrated growth.

FIG. 2 is a graph depicting the differentiation efficiency of WT, HM-KO, and HM-KO-Lenti hESCs measured by NKX6.1 expression levels at stages 4 and 7 of the differentiation protocol.

FIG. 3A is a series of images showing that HM-KO-Lenti stem cell-derived pseudo-islet grafts survive in immunocompetent mice 1 week following transplantations, as shown by GFP IHC.

FIG. 3B is a series of images showing that HM-KO-Lenti stem cell-derived pseudo-islet grafts survive in immunocompetent mice 2 months following transplantations, as shown by GFP IHC.

FIG. 3C shows images of native mammary glands from the same section shown in FIG. 3A and FIG. 3B. The absence of GFP highlights specificity of the signal in FIG. 3A and FIG. 3B.

FIGS. 4A-4D depict schematic representations of PiggyBac plasmid vectors designed for the overexpression of a gene of interest: GPC4 (FIG. 4A), MUC15 (FIG. 4B), SIGLEC14 (FIG. 4C), and TEX10 (FIG. 4D). “ampR” represents ampicillin resistance gene; “pUC ori” represents plasmid University of California origin on replication; “5′ ITR” represents the PiggyBac 5′ inverted terminal repeat; “Kozak” represents the Kozak translation initiation sequence; “rBG pA” represents rabbit beta-globin polyadenylation signal; “CMV” represents human cytomegalovirus immediate early enhancer/promoter; “mCherry-Hygromycin” represents mCherry fused with Hygromycin resistance gene; “BGH pA” represents bovine growth hormone polyadenylation signal; “3′ ITR” represents the PiggyBac 3′ inverted terminal repeat.

FIG. 5 depicts the results of a flow cytometry assessment of the stem cell lines which were engineered to express one of SIGLEC14, TEX10, MUC15, and GPC4. mCherry expression was assessed for each gene, and SIGLEC14 and GPC4 membrane expression was assessed for the respective cell lines.

FIG. 6 is a bar graph summarizing expression of GPC4, MUC15, SIGLEC14, and TEX10 in each of cell lines modified to overexpress the indicated protein. Log fold change was normalized to the non-transfected parental cell line BX1 (“CTRL”).

FIGS. 7A-7E depict fluorescence microscopy images of murine kidney capsule grafts. Embryoid bodies derived from stem cell lines modified to overexpress TEX10 (FIG. 7B), GPC4 (FIG. 7C), SIGLEC14 (FIG. 7D), or MUC15 (FIG. 7E) were grafted under a mouse kidney capsule. The parental cell line BX1 was used as a control (FIG. 7A). After 8 weeks, grafts were harvested and analyzed via confocal fluorescence microscopy.

### DETAILED DESCRIPTION

The disclosure is based, in part, upon the discovery that expression of one of the genes selected from SIGLEC14, MUC15, GPC4, and TEX10 by a mammalian cell can promote evasion of an immune response. Accordingly, the present disclosure provides new, modified mammalian cells (e.g., stem cells or differentiated cells derived therefrom) that are modified with respect to expression of, e.g., one of the following genes or a combination of the following genes: SIGLEC14, MUC15, GPC4, TEX10. The disclosure also provides populations of modified mammalian cells, and tissues comprising and/or derived from such modified cells. Related methods of making and using such modified cells, cell populations, and tissues are also provided.

### I. Definitions

As used herein, the singular forms “a,” “an,” and “the” include plural referents, unless the context clearly dictates otherwise.

As used herein, the term “abrogated,” when used in reference to gene expression, refers to expression that is 1) not detectable, e.g., at an RNA and/or protein level; or 2) below a threshold that corresponds to substantially no gene expression. Such thresholds may be determined by persons of skill in the art and may depend on the gene and/or assay used to determine expression. In some embodiments, abrogated expression in a cell or cell line is made stable over cell replication events, e.g., by a change to the cellular genome. (For example, gene expression may be abrogated by deleting or otherwise rendering inactive an endogenous genetic locus.) In some embodiments, “abrogated” expression in a cell or cell line is transient (e.g., an endogenous locus may be left intact or otherwise functional, and abrogation may depend on a particular condition or absence of condition).

As used herein, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

As used herein, “adult” means post-fetal, e.g., an organism from the neonate stage through the end of life, and includes, for example, cells obtained from delivered placenta tissue, amniotic fluid and/or cord blood.

As used herein, the term “adult differentiated cell” encompasses a wide range of differentiated cell types obtained from an adult organism, that are amenable to producing iPSCs using the instantly described automation system. In some embodiments, the adult differentiated cell is a “fibroblast.” Fibroblasts, also referred to as “fibrocytes” in their less active form, are derived from mesenchyme. Their function includes secreting the precursors of extracellular matrix components including, e.g., collagen. Histologically, fibroblasts are highly branched cells, but fibrocytes are generally smaller and are often described as spindle-shaped. Fibroblasts and fibrocytes derived from any tissue may be employed as a starting material for the automated workflow system on the invention.

Wherever embodiments are described with the language “comprising,” otherwise analogous embodiments described in terms of “consisting of” and/or “consisting essentially of” are included.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

As used herein, the terms “decrease,” “decreased,” “increase,” “increased,” or “reduction,” “reduced,” (e.g., in reference to outcomes or effects) have meanings relative to a reference level. In some embodiments, the reference level is a level as determined by use of comparable conditions, e.g., with a control (such as in an assay, experimental model, or clinical trial).

As used herein, the term “differentiate” or “differentiating,” when used in the context of cell ontogeny, is a relative term that refers to a developmental process by which a cell progresses further down a developmental pathway than its immediate precursor cell.

The term “differentiated cell” encompasses any somatic cell that is not, in its native form, pluripotent, as that term is defined herein. Thus, the term a “differentiated cell” also encompasses cells that are partially differentiated, such as multipotent cells, or cells that are stable, non-pluripotent partially reprogrammed, or partially differentiated cells, generated using any of the compositions and methods described herein. In some embodiments, a differentiated cell is a cell that is a stable intermediate cell, such as a non-pluripotent, partially reprogrammed cell. In some embodiments, the term “differentiated cell” also refers to a cell of a more specialized cell type (e.g., decreased developmental potential) derived from a cell of a less specialized cell type (e.g., increased developmental potential) (e.g., from an undifferentiated cell or a reprogrammed cell) where the cell has undergone a cellular differentiation process.

“Expression vector,” “expression construct,” “plasmid,” or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The terms “heterologous” or “exogenous,” when used to refer to nucleic acids such as DNA, each refer to nucleic acids that originate from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

As used herein, the term “immune rejection” refers to a process by which an intact immune system of a subject, e.g., a mammalian subject, identifies a transplanted cell, tissue, or organ as foreign and triggers a response to damage or destroy the transplanted cell, tissue, or organ. As used herein, the terms “evade,” or “avoid,” when used in the context of the evading or avoiding immune rejection, refers to a foreign cell, tissue, or organ (1) failing to trigger an immune response; or (2) triggering a reduced immune response such that cell, tissue, or organ survives, when the foreign cell, tissue, or organ is transplanted into a subject who has (a) an intact immune system and (b) is not being administered an immunosuppressant. In some embodiments, a cell, tissue, or organ that “evades” or “avoids” immune rejection survives at least for a period long enough to provide therapeutic benefit to the organism.

As used herein, the term “induced pluripotent stem cells” or “iPSCs” refers to stem cells that are produced from differentiated adult cells that have been induced or changed, e.g., reprogrammed into cells capable of differentiating into tissues of all three germ or dermal layers: mesoderm, endoderm, and ectoderm. The term “iPSCs” does not refer to cells found in nature.

As used herein, the term “isolated” when used in conjunction with a particular article (e.g., protein, nucleic acid, or cell) is understood to mean that the article has been separated or purified from other components (e.g., other proteins, nucleic acids, cells, or cellular materials) and/or chemicals (e.g., reagents used in manufacture). Also, the term “isolated” is understood to mean that the article may be separated or purified from the environment in which it may exist in nature, for example, a tissue or fluid sample.

As used herein, the term “modified,” when used in reference to a cell, e.g., a mammalian cell, refers to a non-naturally occurring cell in which one or both of the following is manipulated by the hand of man: (1) the cell's genetic content; or (2) expression of one or more genes or polypeptides (e.g., manipulated by overexpression, reduction, and/or abrogation).

As used herein, the term “normal,” when used in reference to gene expression, is used to refer to gene expression that is approximately the same as that of a reference level. In many embodiments, the reference level is the level of gene expression that is typical from an endogenous locus for the gene, for a similar cell (e.g., of a parental cell line) that has not been artificially modified (e.g., genetically or otherwise manipulated to modulate gene expression). Persons of ordinary skill in the art will typically have access to or be readily able to determine reference levels for various genes.

“Operably-linked” or “functionally linked” refers to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules can be part of a single contiguous nucleic acid molecule and can be adjacent. For example, a promoter is operably-linked to a gene of interest if the promoter regulates or mediates transcription of the gene of interest in a cell.

As used herein, the term “overexpression” is used to refer to gene expression that is increased relative to a reference level. In many embodiments, the reference level is the level of gene expression that is typical from an endogenous locus for the gene, for a similar cell (e.g., of a parental cell line) that has not been artificially modified (e.g., genetically or otherwise manipulated to modulate gene expression). In some embodiments, a gene that is not present in a host cell is overexpressed; thus, in these situations, there is no corresponding endogenous locus for the gene, and the reference level is zero. Persons of ordinary skill in the art will typically have access to or be readily able to determine reference levels for various genes. In some embodiments, overexpression of a gene is made stable through cell replication events, e.g., by a change to the cellular genome. (For example, a gene may be stably overexpressed via an exogenous nucleic acid expression construct that may or may not integrate into the cellular genome and stably replicates with the cell.) In some embodiments, overexpression in a cell or cell line is transient.

In some embodiments, a gene or protein that is overexpressed is overexpressed by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, at least 10-fold, at least 12-fold, at least 14-fold, at least 16-fold, at least 18-fold, at least 20-fold, at least 25-fold, at least 30-fold, at least 40-fold, at least 50-fold, at least 75-fold, at least 100-fold, at least 200-fold, at least 300-fold, at least 400-fold, at least 500-fold, or at least 1,000-fold relative to a reference level.

In some embodiments, a gene or protein that is overexpressed is not normally expressed in the host cell (e.g., the gene may not even be present in the host cell), and the reference level may be approximately zero. In these contexts, “overexpression” refers to any amount of gene expression that is greater than zero.

Within a modified mammalian cell disclosed herein, when multiple genes are overexpressed, each gene that is overexpressed need not be overexpressed by the same amount.

A “promoter” is generally understood as a nucleic acid control sequence that directs transcription of a nucleic acid. An inducible promoter is generally understood as a promoter that mediates transcription of an operably-linked gene in response to a particular stimulus or activating agent (e.g., a doxycycline- or tetracycline-inducible promoter). A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

Exemplary promoters which may be employed in an expression vector of the disclosure include, but are not limited to, the retroviral LTR, the SV40 promoter, the human cytomegalovirus (CMV) promoter, the U6 promoter, and variants thereof. Any other promoter (e.g., cellular promoters such as eukaryotic cellular promoters including, but not limited to, the histone, pol III, j-actin promoters, and variants thereof) can also be employed. Other viral promoters which can be employed include, but are not limited to, adenovirus promoters, TK promoters, B19 parvovirus promoters, and variants thereof.

In certain embodiments, a promoter is an inducible promoter. The use of an inducible promoter allows for expression of an operatively-linked polynucleotide sequence to be turned on or off when desired. In certain embodiments, the promoter is induced in the presence of an exogenous molecule or activity (e.g., a metallothionine promoter, a glucocorticoid promoter, a progesterone promoter, and a tetracycline promoter).

In certain embodiments, a promoter mediates rapid, sustained expression, measured in days (e.g., a CD69 promoter). In certain embodiments, a promoter mediates delayed, late-inducible expression (e.g., a VLA1 promoter). In certain embodiments, a promoter mediates rapid, transient expression (e.g., a TNF promoter, an immediate early response gene promoter and others).

The selection of a promoter (e.g., strong, weak, inducible, tissue-specific, developmental-specific) having specific kinetics of activation (e.g., early and/or late activation), and/or having specific kinetics of expression of an induced gene (e.g., short or long expression) is within the ordinary skill of the artisan and will be apparent to those skilled in the art.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

The terms “stem cell” or “undifferentiated cell,” as used herein, refer to a cell in an undifferentiated or partially differentiated state that has the property of self-renewal and has the developmental potential to differentiate into multiple cell types, without a specific implied meaning regarding developmental potential (e.g., totipotent, pluripotent, and multipotent). A stem cell is capable of proliferation and giving rise to more such stem cells while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

“Wild-type” refers to a gene, protein, cell, virus, or organism found in nature without any known mutation.

### II. Modulated Genes

In various embodiments, one or more genes in mammalian cells disclosed herein are modulated, e.g., such that their expression is overexpressed, reduced, or abrogated. In some embodiments, mammalian cells disclosed herein may be modulated to overexpress one or more genes and have reduced or abrogated expression in one or more other genes.

### Overexpressed Genes

In certain embodiments, modified mammalian cells are modulated to overexpress a gene selected from the group consisting of MUC15, SIGLEC14, TEX10, GPC4, and combinations thereof.

Overexpression of any of the genes or proteins described herein (e.g., MUC15, SIGLEC14, TEX10, GPC4, CD47, CD24, CR1, CD55, CD59, CD46, HLA-G, and/or HLA-E) may be transient, inducible, or stable. Methods of overexpression are known in the art and include, e.g., gene activation systems (e.g., CRISPR-based activation (e.g., using a guide RNA or guide RNAs to target a gene of interest along with a nuclease-inactive Cas enzyme (e.g., dCas9) fused with a transcriptional activator), transfection or transformation with gene expression constructs (e.g., comprising a gene of interest and an operably linked promoter), etc. Viral constructs (e.g., lentiviral constructs, retroviral constructs, or AAV constructs) may be used to effect overexpression of one or more genes.

### MUC15

MUC15 (Mucin 15, Cell Surface Associated) is also known as PAS3 or PASIII. MUC15 encodes Mucin-15, a membrane-bound mucin protein. Mucins are a family of high molecular weight, heavily glycosylated proteins expressed by epithelial cells. Membrane-bound mucins (e.g., Mucin-15) generally have an extracellular domain, a transmembrane domain, and a cytoplasmic domain. The biological function of Mucin-15 is not conventionally understood, although the protein may play a role in cell adhesion to the extracellular matrix. Information regarding MUC15 and exemplary sequences are available, e.g., in the public database from the National Center for Biotechnology Information under Gene ID 143662 (human MUC15).

As used herein, the terms “MUC15”, “MUC15 gene” and related terms are understood to refer to a nucleic acid encoding Mucin-15 protein. Accordingly, as used herein, these terms encompass, e.g., the native MUC15 gene, as well as a protein-coding nucleic acid derived therefrom and/or a cDNA derived therefrom. The terms also encompass, for example, a Mucin-15-encoding nucleic acid that is a component of a vector, e.g., an isolated vector or a vector present in a mammalian cell (optionally wherein the vector is integrated into the genome of the mammalian cell).

In certain embodiments, the present disclosure provides a modified mammalian cell, e.g., a stem cell, which overexpresses MUC15. In certain embodiments, the mammalian cell is modified to overexpress MUC15. In certain embodiments, the mammalian cell comprises a modification (e.g., a genetic modification) for the overexpression of MUC15. In certain embodiments, the cell exogenously expresses MUC15, e.g., from a heterologous locus or DNA segment. In certain embodiments, the cell comprises an expression vector comprising MUC15. In certain embodiments, the expression vector is integrated into the genome of the modified mammalian cell. In certain embodiments, the expression vector is configured for expression, e.g., overexpression, of MUC15 (optionally in combination with one or more other genes). In certain embodiments, MUC15 is human MUC15.

In certain embodiments, the modified mammalian cell (e.g., stem cell) of the disclosure overexpresses Mucin-15. In certain embodiments, the mammalian cell is modified to overexpress Mucin-15. In certain embodiments, the mammalian cell comprises a modification (e.g., a genetic modification) for the overexpression Mucin-15. In certain embodiments, the cell comprises an expression vector encoding Mucin-15. In certain embodiments, the expression vector is configured for expression, e.g., overexpression, of Mucin-15 (optionally in combination with one or more other polypeptides). In certain embodiments, Mucin-15 is human Mucin-15.

In certain embodiments, the modified mammalian cell comprises a Mucin-15-encoding nucleic acid (e.g., a heterologous DNA segment) comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of any of SEQ ID NOs: 1, 3, and 5. In certain embodiments, the modified mammalian cell comprises a Mucin-15-encoding nucleic acid (e.g., a heterologous DNA segment) comprising the nucleotide sequence of any of SEQ ID NOs: 1, 3, and 5. In certain embodiments, the modified mammalian cell comprises a plurality of Mucin-15-encoding nucleic acids, e.g., wherein each of the nucleic acids comprises a nucleotide sequence that is independently selected from the nucleotide sequences of any of SEQ ID NOs: 1, 3, and 5.

In certain embodiments, the modified mammalian cell comprises a nucleic acid (e.g., a heterologous DNA segment) encoding a Mucin-15 polypeptide. In certain embodiments, the modified mammalian cell comprises a nucleic acid (e.g., a heterologous DNA segment) encoding a Mucin-15 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 2, 4, 6, and 7. In certain embodiments, the modified mammalian cell comprises a nucleic acid (e.g., a heterologous DNA segment) encoding a Mucin-15 polypeptide comprising the amino acid sequence of any one of SEQ ID NOs: 2, 4, 6, and 7. In certain embodiments, the modified mammalian cell comprises a plurality of nucleic acids encoding a Mucin-15 polypeptide, e.g., wherein each of the nucleic acids encodes a Mucin-15 polypeptide comprising an amino acid sequence that is independently selected from the amino acid sequences of any of SEQ ID NOs: 2, 4, 6, and 7.

In certain embodiments, the modified mammalian cell comprises an exogenous Mucin-15 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 2, 4, 6, and 7. In certain embodiments, the exogenous polypeptide comprises the amino acid sequence of any one of SEQ ID NOs: 2, 4, 6, and 7. In certain embodiments, the modified mammalian cell comprises a plurality of exogenous Mucin-15 polypeptides, e.g., wherein each of the exogenous Mucin-15 polypeptides comprises an amino acid sequence that is independently selected from the amino acid sequences of any of SEQ ID NOs: 2, 4, 6, and 7.

The disclosure also provides an expression vector comprising a Mucin-15 encoding nucleic acid. In certain embodiments, the expression vector comprises a Mucin-15 encoding nucleic acid comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of any of SEQ ID NOs: 1, 3, and 5. In certain embodiments, the expression vector comprises a Mucin-15-encoding nucleic acid comprising the nucleotide sequence of any of SEQ ID NOs: 1, 3, and 5. In certain embodiments, the expression vector comprises a plurality of Mucin-15-encoding nucleic acids, e.g., wherein each of the Mucin-15-encoding nucleic acids comprises a nucleotide sequence that is independently selected from the nucleotide sequences of any of SEQ ID NOs: 1, 3, and 5. In certain embodiments, the expression vector encodes a Mucin-15 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 2, 4, 6, and 7. In certain embodiments, the expression vector encodes a Mucin-15 polypeptide comprising the amino acid sequence of any one of SEQ ID NOs: 2, 4, 6, and 7. In certain embodiments, the expression vector encodes a plurality of Mucin-15 polypeptides, e.g., wherein each of the Mucin-15 polypeptides comprises an amino acid sequence that is independently selected from the amino acid sequences of any of SEQ ID NOs: 2, 4, 6, and 7.

### SIGLEC14

SIGLEC14 (Sialic Acid Binding Ig Like Lectin 14) encodes the protein Siglec-14. Siglecs are a family of cell surface proteins that bind sialic acid. In general, Siglecs may be found on the surface of immune cells. Siglec-14 has an extracellular domain with three Ig-like domains, and a transmembrane domain with an arginine residue which interacts with ITAM-containing DAP10 and DAP12 proteins. Ligand-binding by Siglec-14 leads to DAP10/12-mediated activation of signaling pathways (e.g., pathways involved in cellular activation). Information regarding SIGLEC14 and exemplary sequences are available, e.g., in the public database from the National Center for Biotechnology Information under Gene ID 100049587 (human SIGLEC14).

As used herein, the terms “SIGLEC14”, “SIGLEC14 gene” and related terms are understood to refer to a nucleic acid encoding Siglec-14 protein. Accordingly, as used herein, these terms encompass, e.g., the native SIGLEC14 gene, as well as a protein-coding nucleic acid derived therefrom and/or a cDNA derived therefrom. The terms also encompass, for example, a Siglec-14-encoding nucleic acid that is a component of a vector, e.g., an isolated vector or a vector present in a mammalian cell (optionally wherein the vector is integrated into the genome of the mammalian cell).

In certain embodiments, the present disclosure provides a modified mammalian cell, e.g., a stem cell, which overexpresses SIGLEC14. In certain embodiments, the mammalian cell is modified to overexpress SIGLEC14. In certain embodiments, the mammalian cell comprises a modification (e.g., a genetic modification) for the overexpression of SIGLEC14. In certain embodiments, the cell exogenously expresses SIGLEC14, e.g., from a heterologous locus or DNA segment. In certain embodiments, the cell comprises an expression vector comprising SIGLEC14. In certain embodiments, the expression vector is integrated into the genome of the modified mammalian cell. In certain embodiments, the expression vector is configured for expression, e.g., overexpression, of SIGLEC14 (optionally in combination with one or more other genes). In certain embodiments, SIGLEC14 is human SIGLEC14.

In certain embodiments, the modified mammalian cell (e.g., stem cell) of the disclosure overexpresses Siglec-14. In certain embodiments, the mammalian cell is modified to overexpress Siglec-14. In certain embodiments, the mammalian cell comprises a modification (e.g., a genetic modification) for the overexpression Siglec-14. In certain embodiments, the cell comprises an expression vector encoding Siglec-14. In certain embodiments, the expression vector is configured for expression, e.g., overexpression, of Siglec-14 (optionally in combination with one or more other polypeptides). In certain embodiments, Siglec-14 is human Siglec-14.

In certain embodiments, the modified mammalian cell comprises a Siglec-14-encoding nucleic acid (e.g., a heterologous DNA segment) comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of SEQ ID NO: 8. In certain embodiments, the modified mammalian cell comprises a Siglec-14-encoding nucleic acid (e.g., a heterologous DNA segment) comprising the nucleotide sequence of SEQ ID NO: 8. In certain embodiments, the modified mammalian cell comprises a plurality of Siglec-14-encoding nucleic acids.

In certain embodiments, the modified mammalian cell comprises a nucleic acid (e.g., a heterologous DNA segment) encoding a Siglec-14 polypeptide. In certain embodiments, the modified mammalian cell comprises a nucleic acid (e.g., a heterologous DNA segment) encoding a Siglec-14 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 9 and 10. In certain embodiments, the modified mammalian cell comprises a nucleic acid (e.g., a heterologous DNA segment) encoding a Siglec-14 polypeptide comprising the amino acid sequence of any one of SEQ ID NOs: 9 and 10. In certain embodiments, the modified mammalian cell comprises a plurality of nucleic acids encoding a Siglec-14 polypeptide, e.g., wherein each of the nucleic acids encodes a Siglec-14 polypeptide comprising an amino acid sequence that is independently selected from the amino acid sequences of any of SEQ ID NOs: 9 and 10.

In certain embodiments, the modified mammalian cell comprises an exogenous Siglec-14 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 9 and 10. In certain embodiments, the exogenous Siglec-14 polypeptide comprises the amino acid sequence of any one of SEQ ID NOs: 9 and 10. In certain embodiments, the modified mammalian cell comprises a plurality of exogenous Siglec-14 polypeptides, e.g., wherein each of the exogenous Siglec-14 polypeptides comprises an amino acid sequence that is independently selected from the amino acid sequences of any of SEQ ID NOs: 9 and 10. The disclosure also provides an expression vector comprising a Siglec-14-encoding nucleic acid. In certain embodiments, the expression vector comprises a nucleic acid comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of SEQ ID NO: 8. In certain embodiments, the expression vector comprises a Siglec-14-encoding nucleic acid comprising the nucleotide sequence of SEQ ID NO: 8. In certain embodiments, the expression vector comprises a plurality of Siglec-14-encoding nucleic acids. In certain embodiments, the expression vector encodes a Siglec-14 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 9 or 10. In certain embodiments, the expression vector encodes a Siglec-14 polypeptide comprising the amino acid sequence of any one of SEQ ID NOs: 9 and 10. In certain embodiments, the expression vector encodes a plurality of Siglec-14 polypeptides, e.g., wherein each of the Siglec-14 polypeptides comprises an amino acid sequence that is independently selected from the amino acid sequences of any of SEQ ID NOs: 9 and 10.

### TEX10

TEX10 (Testis Expressed 10) is also known as Ipi1 and BA208F1.2. TEX10 encodes the protein Testis-expressed Sequence 10 Protein (i.e., Testis-expressed 10 Protein, referred to herein as “Tex10”). Tex10 is conventionally understood to be a component of the Five Friends of Methylatde CHTOP (5FMC) protein complex, which is involved in transcriptional regulation. Tex10 may also be involved in, e.g., RNA degradation, ribosomal RNA (rRNA) processing, and ribosome biogenesis. Information regarding TEX10 and exemplary sequences are available, e.g., in the public database from the National Center for Biotechnology Information under Gene ID 54881 (human TEX10).

As used herein, the terms “TEX10”, “TEX10 gene”, and related terms are understood to refer to a nucleic acid encoding Tex10. Accordingly, as used herein, these terms encompass, e.g., the native TEX10 gene, as well as a protein-coding nucleic acid derived therefrom and/or a cDNA derived therefrom. The terms also encompass, for example, a Tex10-encoding nucleic acid that is a component of a vector, e.g., an isolated vector or a vector present in a mammalian cell (optionally wherein the vector is integrated into the genome of the mammalian cell).

In certain embodiments, the present disclosure provides a modified mammalian cell, e.g., a stem cell, which overexpresses TEX10. In certain embodiments, the mammalian cell is modified to overexpress TEX10. In certain embodiments, the mammalian cell comprises a modification (e.g., a genetic modification) for the overexpression of TEX10. In certain embodiments, the cell exogenously expresses TEX10, e.g., from a heterologous locus or DNA segment. In certain embodiments, the cell comprises an expression vector comprising TEX10. In certain embodiments, the expression vector is integrated into the genome of the modified mammalian cell. In certain embodiments, the expression vector is configured for expression, e.g., overexpression, of TEX10 (optionally in combination with one or more other genes). In certain embodiments, TEX10 is human TEX10.

In certain embodiments, the modified mammalian cell (e.g., stem cell) of the disclosure overexpresses Tex10. In certain embodiments, the mammalian cell is modified to overexpress Tex10. In certain embodiments, the mammalian cell comprises a modification (e.g., a genetic modification) for the overexpression Tex10. In certain embodiments, the cell comprises an expression vector encoding Tex10. In certain embodiments, the expression vector is configured for expression, e.g., overexpression, of Tex10 (optionally in combination with one or more other polypeptides). In certain embodiments, Tex10 is human Tex10.

In certain embodiments, the modified mammalian cell comprises a Tex10-encoding nucleic acid (e.g., a heterologous DNA segment) comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of any of SEQ ID NOs: 11 and 13. In certain embodiments, the modified mammalian cell comprises a Tex10-encoding nucleic acid (e.g., a heterologous DNA segment) comprising the nucleotide sequence of any of SEQ ID NOs: 11 and 13. In certain embodiments, the modified mammalian cell comprises a plurality of Tex10-encoding nucleic acids, e.g., wherein each of the nucleic acids comprises a nucleotide sequence that is independently selected from the nucleotide sequences of any of SEQ ID NOs: 11 and 13.

In certain embodiments, the modified mammalian cell comprises a nucleic acid (e.g., a heterologous DNA segment) encoding a Tex10 polypeptide. In certain embodiments, the modified mammalian cell comprises a nucleic acid (e.g., a heterologous DNA segment) encoding a Tex10 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 12 and 14. In certain embodiments, the modified mammalian cell comprises a nucleic acid (e.g., a heterologous DNA segment) encoding a Tex10 polypeptide comprising the amino acid sequence of any of SEQ ID NOs: 12 and 14. In certain embodiments, the modified mammalian cell comprises a plurality of nucleic acids encoding a Tex10 polypeptide, e.g., wherein each of the nucleic acids encodes a Tex10 polypeptide comprising an amino acid sequence that is independently selected from the amino acid sequences of any of SEQ ID NOs: 12 and 14.

In certain embodiments, the modified mammalian cell comprises an exogenous Tex10 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 12 and 14. In certain embodiments, the exogenous polypeptide comprises the amino acid sequence of any of SEQ ID NOs: 12 and 14. In certain embodiments, the modified mammalian cell comprises a plurality of exogenous Tex10 polypeptides, e.g., wherein each of the exogenous Tex10 polypeptides comprises an amino acid sequence that is independently selected from the amino acid sequences of any of SEQ ID NOs: 12 and 14.

The disclosure also provides an expression vector comprising a Tex10-encoding nucleic acid. In certain embodiments, the expression vector comprises a nucleic acid comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of any of SEQ ID NOs: 11 and 13. In certain embodiments, the expression vector comprises a Tex10-encoding nucleic acid comprising the nucleotide sequence of any of SEQ ID NOs: 11 and 13. In certain embodiments, the expression vector comprises a plurality of Tex10-encoding nucleic acids, e.g., wherein each of the Tex10-encoding nucleic acids comprises a nucleotide sequence that is independently selected from the nucleotide sequences of any of SEQ ID NOs: 11 and 13. In certain embodiments, the expression vector encodes a Tex10 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 12 and 14. In certain embodiments, the expression vector encodes a Tex10 polypeptide comprising the amino acid sequence of any one of SEQ ID NOs: 12 and 14. In certain embodiments, the expression vector encodes a plurality of Tex10 polypeptides, e.g., wherein each of the Tex10 polypeptides comprises an amino acid sequence that is independently selected from the amino acid sequences of any of SEQ ID NOs: 12 and 14.

### GPC4

GPC4 (glypican 4) is also known as K-Glypican and KPTS. GPC4 encodes the protein Glypican-4, a cell-surface heparan sulfate proteoglycan. Cell surface heparan sulfate proteoglycans include a membrane-associated protein core substituted with heparan sulfate chains. Members of the glypican-related integral membrane proteoglycan family (GRIPS) contain a core protein anchored to the cytoplasmic membrane via a glycosyl phosphatidylinositol linkage. GRIPS proteins may play a role in the control of cell division and growth regulation. Information regarding GPC4 and exemplary sequences are available, e.g., in the public database from the National Center for Biotechnology Information under Gene ID 2239 (human GPC4).

As used herein, the terms “GPC4”, “GPC4 gene” and related terms are understood to refer to a nucleic acid encoding Glypican-4 protein. Accordingly, as used herein, these terms encompass, e.g., the native GPC4 gene, as well as a protein-coding nucleic acid derived therefrom and/or a cDNA derived therefrom. The terms also encompass, for example, a Glypican-4-encoding nucleic acid that is a component of a vector, e.g., an isolated vector or a vector present in a mammalian cell (optionally wherein the vector is integrated into the genome of the mammalian cell).

In certain embodiments, the present disclosure provides a modified mammalian cell, e.g., a stem cell, which overexpresses GPC4. In certain embodiments, the mammalian cell is modified to overexpress GPC4. In certain embodiments, the mammalian cell comprises a modification (e.g., a genetic modification) for the overexpression of GPC4. In certain embodiments, the cell exogenously expresses GPC4, e.g., from a heterologous locus or DNA segment. In certain embodiments, the cell comprises an expression vector comprising GPC4. In certain embodiments, the expression vector is integrated into the genome of the modified mammalian cell. In certain embodiments, the expression vector is configured for expression, e.g., overexpression, of GPC4 (optionally in combination with one or more other genes). In certain embodiments, GPC4 is human GPC4.

In certain embodiments, the modified mammalian cell (e.g., stem cell) of the disclosure overexpresses Glypican-4. In certain embodiments, the mammalian cell is modified to overexpress Glypican-4. In certain embodiments, the mammalian cell comprises a modification (e.g., a genetic modification) for the overexpression Glypican-4. In certain embodiments, the cell comprises an expression vector encoding Glypican-4. In certain embodiments, the expression vector is configured for expression, e.g., overexpression, of Glypican-4 (optionally in combination with one or more other polypeptides). In certain embodiments, Glypican-4 is human Glypican-4.

In certain embodiments, the modified mammalian cell comprises a Glypican-4-encoding nucleic acid (e.g., a heterologous DNA segment) comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of SEQ ID NO: 15. In certain embodiments, the modified mammalian cell comprises a Glypican-4-encoding nucleic acid (e.g., a heterologous DNA segment) comprising the nucleotide sequence of SEQ ID NO: 15. In certain embodiments, the modified mammalian cell comprises a plurality of Glypican-4-encoding nucleic acids.

In certain embodiments, the modified mammalian cell comprises a nucleic acid (e.g., a heterologous DNA segment) encoding a Glypican-4 polypeptide. In certain embodiments, the modified mammalian cell comprises a nucleic acid (e.g., a heterologous DNA segment) encoding a Glypican-4 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 16 and 17. In certain embodiments, the modified mammalian cell comprises a nucleic acid (e.g., a heterologous DNA segment) encoding a Glypican-4 polypeptide comprising the amino acid sequence of any of SEQ ID NOs: 16 and 17. In certain embodiments, the modified mammalian cell comprises a plurality of nucleic acids encoding a Glypican-4 polypeptide, e.g., wherein each of the nucleic acids encodes a Glypican-4 polypeptide comprising an amino acid sequence that is independently selected from the amino acid sequences of any of SEQ ID NOs: 16 and 17.

In certain embodiments, the modified mammalian cell comprises an exogenous Glypican-4 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 16 and 17. In certain embodiments, the exogenous polypeptide comprises the amino acid sequence of any of SEQ ID NOs: 16 and 17. In certain embodiments, the modified mammalian cell comprises a plurality of exogenous Glypican-4 polypeptides, e.g., wherein each of the exogenous Glypican-4 polypeptides comprises an amino acid sequence that is independently selected from the amino acid sequences of any of SEQ ID NOs: 16 and 17.

The disclosure also provides an expression vector comprising a Glypican-4-encoding nucleic acid. In certain embodiments, the expression vector comprises a nucleic acid comprising a nucleotide sequence having at least 85% identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) to the nucleotide sequence of SEQ ID NO: 15. In certain embodiments, the expression vector comprises a Glypican-4-encoding nucleic acid comprising the nucleotide sequence of SEQ ID NO: 15. In certain embodiments, the expression vector comprises a plurality of Glypican-4-encoding nucleic acids. In certain embodiments, the expression vector encodes a Glypican-4 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 16 and 17. In certain embodiments, the expression vector encodes a Glypican-4 polypeptide comprising the amino acid sequence of any of SEQ ID NOs: 16 and 17. In certain embodiments, the expression vector encodes a plurality of Glypican-4 polypeptides, e.g., wherein each of the Glypican-4 polypeptides comprises an amino acid sequence that is independently selected from the amino acid sequences of any of SEQ ID NOs: 16 and 17.

### Additional Overexpressed Genes

In certain embodiments, mammalian genes are modulated to overexpress one or more genes associated with inhibition of macrophage phagocytosis (e.g., CD47 and/or CD24), one or more genes associated with inhibition of complement activation and deposition (e.g., CD55, CD59, CD46, and/or CR1), and/or one or more genes associated with inhibition of NK cell activation (e.g., HLA-G and/or HLA-E).

### CD47

CD47 (CD47 Molecule) is also known as IAP, OA3, or MER6. CD47 encodes the protein CD47 (i.e., Cluster of Differentiation 47 or Leukocyte surface antigen CD47, or Integrin Associated Protein), which is believed to have a role in cell proliferation, cell death, cell migration, angiogenesis, and inflammation. Additionally, without wishing to be bound by theory, CD47 is also understood to inhibit phagocytosis by macrophages. Information regarding CD47 and exemplary sequences are available, e.g., in the public database from the National Center for Biotechnology Information under Gene ID 961 (human CD47).

As used herein, the terms “CD47”, “CD47 gene”, and related terms are understood to refer to a nucleic acid encoding CD47 protein. Accordingly, as used herein, these terms encompass, e.g., the native CD47 gene, as well as a protein-coding nucleic acid derived therefrom and/or a cDNA derived therefrom. The terms also encompass, for example, a CD47-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CD47. In certain embodiments, the mammalian cell is modified to overexpress CD47. In certain embodiments, the mammalian cell comprises a modification (e.g., a genetic modification) for the overexpression of CD47. In certain embodiments, the cell exogenously expresses CD47, e.g., from a heterologous locus or DNA segment. In certain embodiments, the cell comprises an expression vector comprising CD47. In certain embodiments, the expression vector is integrated into the genome of the modified mammalian cell. In certain embodiments, the expression vector is configured for expression, e.g., overexpression, of CD47 (optionally in combination with one or more other genes). In certain embodiments, CD47 is human CD47.

In certain embodiments, the modified mammalian cell (e.g., stem cell) of the disclosure overexpresses CD47 polypeptide. In certain embodiments, the mammalian cell is modified to overexpress CD47 polypeptide. In certain embodiments, the mammalian cell comprises a modification (e.g., a genetic modification) for the overexpression CD47 polypeptide. In certain embodiments, the cell comprises an expression vector encoding CD47 polypeptide. In certain embodiments, the expression vector is configured for expression, e.g., overexpression, of CD47 polypeptide (optionally in combination with one or more other polypeptides). In certain embodiments, the CD47 polypeptide is human CD47.

In certain embodiments, the modified mammalian cell comprises a nucleic acid (e.g., a heterologous DNA segment) encoding a CD47 polypeptide. In certain embodiments, the modified mammalian cell comprises a nucleic acid (e.g., a heterologous DNA segment) encoding a CD47 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 18-25. In certain embodiments, the modified mammalian cell comprises a nucleic acid (e.g., a heterologous DNA segment) encoding a CD47 polypeptide comprising the amino acid sequence of any of SEQ ID NOs: 18-25.

In certain embodiments, the modified mammalian cell comprises an exogenous CD47 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 18-25. In certain embodiments, exogenous polypeptide comprises the amino acid sequence of any of SEQ ID NOs: 18-25.

The disclosure also provides an expression vector comprising a CD47-encoding nucleic acid. In certain embodiments, the expression vector encodes a CD47 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 18-25. In certain embodiments, the expression vector encodes a CD47 polypeptide comprising the amino acid sequence of any of SEQ ID NOs: 18-25.

### CD24

CD24 (CD24 Molecule) is also known as CD24A. CD24 encodes the protein CD24 (i.e., Cluster of Differentiation 24) which is believed to have a role in cell adhesion. Additionally, without wishing to be bound by theory, CD24 is also believed to have a role in inhibiting phagocytosis by macrophages. Information regarding CD24 and exemplary sequences are available, e.g., in the public database from the National Center for Biotechnology Information under Gene ID 100133941 (human CD24).

As used herein, the terms “CD24”, “CD24 gene”, and related terms are understood to refer to a nucleic acid encoding CD24 protein. Accordingly, as used herein, these terms encompass, e.g., the native CD24 gene, as well as a protein-coding nucleic acid derived therefrom and/or a cDNA derived therefrom. The terms also encompass, for example, a CD24-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CD24. In certain embodiments, the mammalian cell is modified to overexpress CD24. In certain embodiments, the mammalian cell comprises a modification (e.g., a genetic modification) for the overexpression of CD24. In certain embodiments, the cell exogenously expresses CD24, e.g., from a heterologous locus or DNA segment. In certain embodiments, the cell comprises an expression vector comprising CD24. In certain embodiments, the expression vector is integrated into the genome of the modified mammalian cell. In certain embodiments, the expression vector is configured for expression, e.g., overexpression, of CD24 (optionally in combination with one or more other genes). In certain embodiments, CD24 is human CD24.

In certain embodiments, the modified mammalian cell (e.g., stem cell) of the disclosure overexpresses CD24 polypeptide. In certain embodiments, the mammalian cell is modified to overexpress CD24 polypeptide. In certain embodiments, the mammalian cell comprises a modification (e.g., a genetic modification) for the overexpression CD24 polypeptide. In certain embodiments, the cell comprises an expression vector encoding CD24 polypeptide. In certain embodiments, the expression vector is configured for expression, e.g., overexpression, of CD24 polypeptide (optionally in combination with one or more other polypeptides). In certain embodiments, the CD24 polypeptide is human CD24.

In certain embodiments, the modified mammalian cell comprises a nucleic acid (e.g., a heterologous DNA segment) encoding a CD24 polypeptide. In certain embodiments, the modified mammalian cell comprises a nucleic acid (e.g., a heterologous DNA segment) encoding a CD24 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 26-28. In certain embodiments, the modified mammalian cell comprises a nucleic acid (e.g., a heterologous DNA segment) encoding a CD24 polypeptide comprising the amino acid sequence of any of SEQ ID NOs: 26-28.

In certain embodiments, the modified mammalian cell comprises an exogenous CD24 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 26-28. In certain embodiments, exogenous polypeptide comprises the amino acid sequence of any of SEQ ID NOs: 26-28.

The disclosure also provides an expression vector comprising a CD24-encoding nucleic acid. In certain embodiments, the expression vector encodes a CD24 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 26-28. In certain embodiments, the expression vector encodes a CD24 polypeptide comprising the amino acid sequence of any of SEQ ID NOs: 26-28.

### CR1

CR1 (Complement C3b C4b Receptor 1 (Knops Blood Group)) is also known as, e.g., KN, C3BR, C4BR, and CD35. CR1 encodes the protein CR1 (Complement Receptor 1, or CD35) which is understood to have a role in, e.g., inhibiting the complement cascade. Information regarding CR1 and exemplary sequences are available, e.g., in the public database from the National Center for Biotechnology Information under Gene ID 1378 (human CR1).

As used herein, the terms “CR1”, “CR1 gene”, and related terms are understood to refer to a nucleic acid encoding CR1 protein. Accordingly, as used herein, these terms encompass, e.g., the native CR1 gene, as well as a protein-coding nucleic acid derived therefrom and/or a cDNA derived therefrom. The terms also encompass, for example, a CR1-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CR1. In certain embodiments, the mammalian cell is modified to overexpress CR1. In certain embodiments, the mammalian cell comprises a modification (e.g., a genetic modification) for the overexpression of CR1. In certain embodiments, the cell exogenously expresses CR1, e.g., from a heterologous locus or DNA segment. In certain embodiments, the cell comprises an expression vector comprising CR1. In certain embodiments, the expression vector is integrated into the genome of the modified mammalian cell. In certain embodiments, the expression vector is configured for expression, e.g., overexpression, of CR1 (optionally in combination with one or more other genes). In certain embodiments, CR1 is human CR1.

In certain embodiments, the modified mammalian cell (e.g., stem cell) of the disclosure overexpresses CR1 polypeptide. In certain embodiments, the mammalian cell is modified to overexpress CR1 polypeptide. In certain embodiments, the mammalian cell comprises a modification (e.g., a genetic modification) for the overexpression of CR1 polypeptide. In certain embodiments, the cell comprises an expression vector encoding CR1 polypeptide. In certain embodiments, the expression vector is configured for expression, e.g., overexpression, of CR1 polypeptide (optionally in combination with one or more other polypeptides). In certain embodiments, the CR1 polypeptide is human CR1.

In certain embodiments, the modified mammalian cell comprises a nucleic acid (e.g., a heterologous DNA segment) encoding a CR1 polypeptide. In certain embodiments, the modified mammalian cell comprises a nucleic acid (e.g., a heterologous DNA segment) encoding a CR1 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 39 and 40. In certain embodiments, the modified mammalian cell comprises a nucleic acid (e.g., a heterologous DNA segment) encoding a CR1 polypeptide comprising the amino acid sequence of any of SEQ ID NOs: 39 and 40.

In certain embodiments, the modified mammalian cell comprises an exogenous CR1 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 39 and 40. In certain embodiments, exogenous polypeptide comprises the amino acid sequence of any of SEQ ID NOs: 39 and 40.

The disclosure also provides an expression vector comprising a CR1-encoding nucleic acid. In certain embodiments, the expression vector encodes a CR1 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 39 and 40. In certain embodiments, the expression vector encodes a CR1 polypeptide comprising the amino acid sequence of any of SEQ ID NOs: 39 and 40.

### CD55

CD55 (CD55 Molecule (Cromer blood group)) is also known as CR, TC, DAF, CROM, and CHAPLE. CD55 encodes the protein CD55 (i.e., Complement Decay-accelerating Factor, or DAF), which is believed to interact with complement proteins to disrupt the complement cascade and inhibit formation of the membrane attack complex. Information regarding CD55 and exemplary sequences are available, e.g., in the public database from the National Center for Biotechnology Information under Gene ID 1604 (human CD55).

As used herein, the terms “CD55”, “CD55 gene”, and related terms are understood to refer to a nucleic acid encoding CD55 protein. Accordingly, as used herein, these terms encompass, e.g., the native CD55 gene, as well as a protein-coding nucleic acid derived therefrom and/or a cDNA derived therefrom. The terms also encompass, for example, a CD55-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CD55. In certain embodiments, the mammalian cell is modified to overexpress CD55. In certain embodiments, the mammalian cell comprises a modification (e.g., a genetic modification) for the overexpression of CD55. In certain embodiments, the cell exogenously expresses CD55, e.g., from a heterologous locus or DNA segment. In certain embodiments, the cell comprises an expression vector comprising CD55. In certain embodiments, the expression vector is integrated into the genome of the modified mammalian cell. In certain embodiments, the expression vector is configured for expression, e.g., overexpression, of CD55 (optionally in combination with one or more other genes). In certain embodiments, CD55 is human CD55.

In certain embodiments, the modified mammalian cell (e.g., stem cell) of the disclosure overexpresses CD55 polypeptide. In certain embodiments, the mammalian cell is modified to overexpress CD55 polypeptide. In certain embodiments, the mammalian cell comprises a modification (e.g., a genetic modification) for the overexpression of CD55 polypeptide. In certain embodiments, the cell comprises an expression vector encoding CD55 polypeptide. In certain embodiments, the expression vector is configured for expression, e.g., overexpression, of CD55 polypeptide (optionally in combination with one or more other polypeptides). In certain embodiments, the CD55 polypeptide is human CD55.

In certain embodiments, the modified mammalian cell comprises a nucleic acid (e.g., a heterologous DNA segment) encoding a CD55 polypeptide. In certain embodiments, the modified mammalian cell comprises a nucleic acid (e.g., a heterologous DNA segment) encoding a CD55 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 29 and 30 or to the amino acid sequence set forth in any of NCBI Ref Nos. NP_001108224.1, NP_001287832.1, NP_001287833.1, and NP_001287831.1. In certain embodiments, the modified mammalian cell comprises a nucleic acid (e.g., a heterologous DNA segment) encoding a CD55 polypeptide comprising the amino acid sequence of any of SEQ ID NOs: 29 and 30 or the amino acid sequence set forth in any of NCBI Ref. Nos. NP_001108224.1, NP_001287832.1, NP_001287833.1, and NP_001287831.1.

In certain embodiments, the modified mammalian cell comprises an exogenous CD55 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 29 and 30 or to the amino acid sequence set forth in any of NCBI Ref Nos. NP_001108224.1, NP_001287832.1, NP_001287833.1, and NP_001287831.1. In certain embodiments, the exogenous polypeptide comprises the amino acid sequence of any of SEQ ID NOs: 29 and 30 or the amino acid sequence set forth in any of NCBI Ref. Nos. NP_001108224.1, NP_001287832.1, NP_001287833.1, and NP_001287831.1.

The disclosure also provides an expression vector comprising a CD55-encoding nucleic acid. In certain embodiments, the expression vector encodes a CD55 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 29 and 30 or to the amino acid sequence set forth in any of NCBI Ref. Nos. NP_001108224.1, NP 001287832.1, NP 001287833.1, and NP_001287831.1. In certain embodiments, the expression vector encodes a CD55 polypeptide comprising the amino acid sequence of any of SEQ ID NOs: 29 and 30 or the amino acid sequence set forth in any of NCBI Ref. Nos. NP_001108224.1, NP 001287832.1, NP_001287833.1, and NP_001287831.1.

### CD59

CD59 (CD59 Molecule (CD59 blood group)) is also known as, e.g., MACIF and MAC-IP. CD59 encodes the protein CD59, which is believed to inhibit formation of the complement membrane attack complex. Information regarding CD59 and exemplary sequences are available, e.g., in the public database from the National Center for Biotechnology Information under Gene ID 966 (human CD59).

As used herein, the terms “CD59”, “CD59 gene”, and related terms are understood to refer to a nucleic acid encoding CD59 protein. Accordingly, as used herein, these terms encompass, e.g., the native CD59 gene, as well as a protein-coding nucleic acid derived therefrom and/or a cDNA derived therefrom. The terms also encompass, for example, a CD59-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CD59. In certain embodiments, the mammalian cell is modified to overexpress CD59. In certain embodiments, the mammalian cell comprises a modification (e.g., a genetic modification) for the overexpression of CD59. In certain embodiments, the cell exogenously expresses CD59, e.g., from a heterologous locus or DNA segment. In certain embodiments, the cell comprises an expression vector comprising CD59. In certain embodiments, the expression vector is integrated into the genome of the modified mammalian cell. In certain embodiments, the expression vector is configured for expression, e.g., overexpression, of CD59 (optionally in combination with one or more other genes). In certain embodiments, CD59 is human CD59.

In certain embodiments, the modified mammalian cell (e.g., stem cell) of the disclosure overexpresses CD59 polypeptide. In certain embodiments, the mammalian cell is modified to overexpress CD59 polypeptide. In certain embodiments, the mammalian cell comprises a modification (e.g., a genetic modification) for the overexpression of CD59 polypeptide. In certain embodiments, the cell comprises an expression vector encoding CD59 polypeptide. In certain embodiments, the expression vector is configured for expression, e.g., overexpression, of CD59 polypeptide (optionally in combination with one or more other polypeptides). In certain embodiments, the CD59 polypeptide is human CD59.

In certain embodiments, the modified mammalian cell comprises a nucleic acid (e.g., a heterologous DNA segment) encoding a CD59 polypeptide. In certain embodiments, the modified mammalian cell comprises a nucleic acid (e.g., a heterologous DNA segment) encoding a CD59 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 31 and 32. In certain embodiments, the modified mammalian cell comprises a nucleic acid (e.g., a heterologous DNA segment) encoding a CD59 polypeptide comprising the amino acid sequence of any of SEQ ID NOs: 31 and 32.

In certain embodiments, the modified mammalian cell comprises an exogenous CD59 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 31 and 32. In certain embodiments, exogenous polypeptide comprises the amino acid sequence of any of SEQ ID NOs: 31 and 32.

The disclosure also provides an expression vector comprising a CD59-encoding nucleic acid. In certain embodiments, the expression vector encodes a CD59 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 31 and 32. In certain embodiments, the expression vector encodes a CD59 polypeptide comprising the amino acid sequence of any of SEQ ID NOs: 31 and 32.

### CD46

CD46 (CD46 Molecule) is also known as MCP, TLX, AHUS2, MIC10, and TRA2.10. CD46 encodes the protein CD46 (i.e., Cluster of Differentiation 46, or Membrane Cofactor Protein), which is believed to be an inhibitory complement receptor. Information regarding CD46 and exemplary sequences are available, e.g., in the public database from the National Center for Biotechnology Information under Gene ID 4179 (human CD46). The murine gene Crry is a homolog of CD46.

As used herein, the terms “CD46”, “CD46 gene”, and related terms are understood to refer to a nucleic acid encoding CD46 protein. Accordingly, as used herein, these terms encompass, e.g., the native CD46 gene, as well as a protein-coding nucleic acid derived therefrom and/or a cDNA derived therefrom. The terms also encompass, for example, a CD46-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses CD46. In certain embodiments, the mammalian cell is modified to overexpress CD46. In certain embodiments, the mammalian cell comprises a modification (e.g., a genetic modification) for the overexpression of CD46. In certain embodiments, the cell exogenously expresses CD46, e.g., from a heterologous locus or DNA segment. In certain embodiments, the cell comprises an expression vector comprising CD46. In certain embodiments, the expression vector is integrated into the genome of the modified mammalian cell. In certain embodiments, the expression vector is configured for expression, e.g., overexpression, of CD46 (optionally in combination with one or more other genes). In certain embodiments, CD46 is human CD46.

In certain embodiments, the modified mammalian cell (e.g., stem cell) of the disclosure overexpresses CD46 polypeptide. In certain embodiments, the mammalian cell is modified to overexpress CD46 polypeptide. In certain embodiments, the mammalian cell comprises a modification (e.g., a genetic modification) for the overexpression of CD46 polypeptide. In certain embodiments, the cell comprises an expression vector encoding CD46 polypeptide. In certain embodiments, the expression vector is configured for expression, e.g., overexpression, of CD46 polypeptide (optionally in combination with one or more other polypeptides). In certain embodiments, the CD46 polypeptide is human CD46.

In certain embodiments, the modified mammalian cell comprises a nucleic acid (e.g., a heterologous DNA segment) encoding a CD46 polypeptide. In certain embodiments, the modified mammalian cell comprises a nucleic acid (e.g., a heterologous DNA segment) encoding a CD46 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 33 and 34 or to the amino acid sequence set forth in any of NCBI Ref. Nos. XP_011507865.1, XP_047276844.1, XP_047276850.1, XP_047276857.1, XP 047276865.1, NP_758869.1, NP 758861.1, NP_722548.1, NP 758868.1, NP_758865.1, NP_758866.1, NP 758862.1, NP_758863.1, NP 758860.1, NP_758867.1, and NP_758871.1. In certain embodiments, the modified mammalian cell comprises a nucleic acid (e.g., a heterologous DNA segment) encoding a CD46 polypeptide comprising the amino acid sequence of any of SEQ ID NOs: 33 and 34 or the amino acid sequence set forth in any of NCBI Ref. Nos. XP 011507865.1, XP_047276844.1, XP_047276850.1, XP 047276857.1, XP 047276865.1, NP_758869.1, NP 758861.1, NP_722548.1, NP_758868.1, NP_758865.1, NP_758866.1, NP_758862.1, NP_758863.1, NP_758860.1, NP_758867.1, and NP_758871.1.

In certain embodiments, the modified mammalian cell comprises an exogenous CD46 polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 33 and 34 or to the amino acid sequence set forth in any of NCBI Ref. Nos. XP_011507865.1, XP_047276844.1, XP 047276850.1, XP 047276857.1, XP_047276865.1, NP 758869.1, NP_758861.1, NP_722548.1, NP 758868.1, NP_758865.1, NP_758866.1, NP_758862.1, NP_758863.1, NP_758860.1, NP_758867.1, and NP_758871.1. In certain embodiments, the exogenous polypeptide comprises the amino acid sequence of any of SEQ ID NOs: 33 and 34 or the amino acid sequence set forth in any of NCBI Ref. Nos. XP_011507865.1, XP_047276844.1, XP 047276850.1, XP 047276857.1, XP_047276865.1, NP 758869.1, NP_758861.1, NP_722548.1, NP 758868.1, NP_758865.1, NP_758866.1, NP_758862.1, NP_758863.1, NP_758860.1, NP_758867.1, and NP_758871.1.

The disclosure also provides an expression vector comprising a CD46-encoding nucleic acid. In certain embodiments, the expression vector encodes a CD46 polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 33 and 34 or to the amino acid sequence set forth in any of NCBI Ref. Nos. XP_011507865.1, XP 047276844.1, XP 047276850.1, XP_047276857.1, XP_047276865.1, NP_758869.1, NP_758861.1, NP_722548.1, NP_758868.1, NP_758865.1, NP_758866.1, NP_758862.1, NP_758863.1, NP_758860.1, NP_758867.1, and NP_758871.1. In certain embodiments, the expression vector encodes a CD55 polypeptide comprising the amino acid sequence of any of SEQ ID NOs: 33 and 34 or the amino acid sequence set forth in any of NCBI Ref Nos. XP 011507865.1, XP 047276844.1, XP_047276850.1, XP_047276857.1, XP_047276865.1, NP_758869.1, NP_758861.1, NP_722548.1, NP 758868.1, NP_758865.1, NP_758866.1, NP_758862.1, NP_758863.1, NP_758860.1, NP_758867.1, and NP_758871.1.

### HLA-G

HLA-G (Major Histocompatibility Complex, Class I, G) is also known as MHCG. HLA-G encodes the protein HLA-G (i.e., HLA class I histocompatibility antigen, alpha chain G), which is understood to be a nonclassical HLA heavy chain protein, which can function as an immune checkpoint protein and inhibit NK cell activation. Information regarding HLA-G and exemplary sequences are available, e.g., in the public database from the National Center for Biotechnology Information under Gene ID 3135 (human HLA-G). The murine gene K^{b} is a homolog of HLA-G.

As used herein, the terms “HLA-G”, “HLA-G gene”, and related terms are understood to refer to a nucleic acid encoding HLA-G protein. Accordingly, as used herein, these terms encompass, e.g., the native HLA-G gene, as well as a protein-coding nucleic acid derived therefrom and/or a cDNA derived therefrom. The terms also encompass, for example, a nucleic acid encoding an HLA-G single-chain trimer, e.g., as described in Hansen et al., “Translational and basic applications of peptide-MHCI single chain trimers”, Trends Immunol. (2010) 31(10): 363-369, which is hereby incorporated by reference in its entirety. The terms also encompass, for example, an HLA-G-encoding nucleic acid or an HLA-G single-chain trimer-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses HLA-G. In certain embodiments, the mammalian cell is modified to overexpress HLA-G. In certain embodiments, the mammalian cell comprises a modification (e.g., a genetic modification) for the overexpression of HLA-G. In certain embodiments, the cell exogenously expresses HLA-G, e.g., from a heterologous locus or DNA segment. In certain embodiments, the cell comprises an expression vector comprising HLA-G. In certain embodiments, the expression vector is integrated into the genome of the modified mammalian cell. In certain embodiments, the expression vector is configured for expression, e.g., overexpression, of HLA-G (optionally in combination with one or more other genes). In certain embodiments, HLA-G is human HLA-G.

In certain embodiments, the modified mammalian cell (e.g., stem cell) of the disclosure overexpresses HLA-G polypeptide or an HLA-G single-chain trimer. In certain embodiments, the mammalian cell is modified to overexpress HLA-G polypeptide or an HLA-G single-chain trimer. In certain embodiments, the mammalian cell comprises a modification (e.g., a genetic modification) for the overexpression HLA-G polypeptide or an HLA-G single-chain trimer. In certain embodiments, the cell comprises an expression vector encoding HLA-G polypeptide or an HLA-G single-chain trimer. In certain embodiments, the expression vector is configured for expression, e.g., overexpression, of HLA-G polypeptide or an HLA-G single-chain trimer (optionally in combination with one or more other polypeptides). In certain embodiments, the HLA-G polypeptide is human HLA-G. In certain embodiments, the HLA-G single-chain trimer comprises human HLA-G.

In certain embodiments, the modified mammalian cell comprises a nucleic acid (e.g., a heterologous DNA segment) encoding an HLA-G polypeptide or an HLA-G single-chain trimer polypeptide. In certain embodiments, the modified mammalian cell comprises a nucleic acid (e.g., a heterologous DNA segment) encoding a HLA-G polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 35 and 36. In certain embodiments, the modified mammalian cell comprises a nucleic acid (e.g., a heterologous DNA segment) encoding an HLA-G polypeptide comprising the amino acid sequence of any of SEQ ID NOs: 35 and 36.

In certain embodiments, the modified mammalian cell comprises an exogenous HLA-G polypeptide or an exogenous HLA-G single-chain trimer polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 35 and 36. In certain embodiments, exogenous polypeptide comprises the amino acid sequence of any of SEQ ID NOs: 35 and 36.

The disclosure also provides an expression vector comprising an HLA-G-encoding or HLA-G single-chain trimer-encoding nucleic acid. In certain embodiments, the expression vector encodes an HLA-G polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 35 and 36. In certain embodiments, the expression vector encodes an HLA-G polypeptide comprising the amino acid sequence of any of SEQ ID NOs: 35 and 36.

### HLA-E

HLA-E (Major Histocompatibility Complex, Class I, E) is also known as QA1 and HLA-6.2. HLA-E encodes the protein HLA-E (i.e., HLA class I histocompatibility antigen, alpha chain E), which is understood to be a nonclassical HLA heavy chain protein that can inhibit NK cell activation. Information regarding HLA-E and exemplary sequences are available, e.g., in the public database from the National Center for Biotechnology Information under Gene ID 3133 (human HLA-E). The murine gene Qa1 is a homolog of HLA-E.

As used herein, the terms “HLA-E”, “HLA-E gene”, and related terms are understood to refer to a nucleic acid encoding HLA-E protein. Accordingly, as used herein, these terms encompass, e.g., the native HLA-E gene, as well as a protein-coding nucleic acid derived therefrom and/or a cDNA derived therefrom. The terms also encompass, for example, a nucleic acid encoding an HLA-E single-chain trimer, e.g., as described in Hansen et al., supra. The terms also encompass, for example, an HLA-E-encoding nucleic acid or an HLA-E single-chain trimer-encoding nucleic acid that is a component of an expression vector, e.g., an isolated expression vector or an expression vector present in a mammalian cell (optionally wherein the expression vector is integrated into the genome of the mammalian cell).

In certain embodiments, the present disclosure provides a modified mammalian cell, e.g., a stem cell, which overexpresses HLA-E. In certain embodiments, the mammalian cell is modified to overexpress HLA-E. In certain embodiments, the mammalian cell comprises a modification (e.g., a genetic modification) for the overexpression of HLA-E. In certain embodiments, the cell exogenously expresses HLA-E, e.g., from a heterologous locus or DNA segment. In certain embodiments, the cell comprises an expression vector comprising HLA-G. In certain embodiments, the expression vector is integrated into the genome of the modified mammalian cell. In certain embodiments, the expression vector is configured for expression, e.g., overexpression, of HLA-E (optionally in combination with one or more other genes). In certain embodiments, HLA-E is human HLA-E.

In certain embodiments, the modified mammalian cell (e.g., stem cell) of the disclosure overexpresses HLA-E polypeptide or an HLA-E single-chain trimer. In certain embodiments, the mammalian cell is modified to overexpress HLA-E polypeptide or an HLA-E single-chain trimer. In certain embodiments, the mammalian cell comprises a modification (e.g., a genetic modification) for the overexpression HLA-E polypeptide or an HLA-E single-chain trimer. In certain embodiments, the cell comprises an expression vector encoding HLA-E polypeptide or an HLA-E single-chain trimer. In certain embodiments, the expression vector is configured for expression, e.g., overexpression, of HLA-E polypeptide or an HLA-E single-chain trimer (optionally in combination with one or more other polypeptides). In certain embodiments, the HLA-E polypeptide is human HLA-E. In certain embodiments, HLA-E single-chain trimer comprises human HLA-E.

In certain embodiments, the modified mammalian cell comprises a nucleic acid (e.g., a heterologous DNA segment) encoding an HLA-E polypeptide or an HLA-E single-chain trimer polypeptide. In certain embodiments, the modified mammalian cell comprises a nucleic acid (e.g., a heterologous DNA segment) encoding a HLA-E polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 37 and 38. In certain embodiments, the modified mammalian cell comprises a nucleic acid (e.g., a heterologous DNA segment) encoding an HLA-G polypeptide comprising the amino acid sequence of any of SEQ ID NOs: 37 and 38.

In certain embodiments, the modified mammalian cell comprises an exogenous HLA-G polypeptide or an exogenous HLA-G single-chain trimer polypeptide. In certain embodiments, the exogenous polypeptide comprises an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any one of SEQ ID NOs: 35 and 36. In certain embodiments, exogenous polypeptide comprises the amino acid sequence of any of SEQ ID NOs: 37 and 38.

The disclosure also provides an expression vector comprising an HLA-G-encoding or HLA-G single-chain trimer-encoding nucleic acid. In certain embodiments, the expression vector encodes an HLA-G polypeptide comprising an amino acid sequence having at least 90% identity (e.g., at least 91%, at least 92%, at least 93%, at least 94%, at least 95%, at least 96%, at least 97%, at least 98%, or at least 99% sequence identity) to the amino acid sequence of any of SEQ ID NOs: 37 and 38. In certain embodiments, the expression vector encodes an HLA-G polypeptide comprising the amino acid sequence of any of SEQ ID NOs: 37 and 38.

Overexpression may be transient, inducible, or stable. Methods of overexpression are known in the art and include, e.g., gene activation systems (e.g., CRISPR-based activation (e.g., using a guide RNA or guide RNAs to target a gene of interest along with a nuclease-inactive Cas enzyme (e.g., dCas9) fused with a transcriptional activator), transfection or transformation with gene expression constructs (e.g., comprising a gene of interest and an operably linked promoter), etc. Viral constructs (e.g., lentiviral constructs, retroviral constructs, or AAV constructs) may be used to effect overexpression of one or more genes.

### Genes Whose Expression May be Reduced or Abrogated

In certain embodiments, a mammalian cell is modulated such that expression of one or more genes is reduced or abrogated.

For example, mammalian cells disclosed herein may be modulated to have reduced or abrogated expression in one or more genes associated with T-cell activation. Examples of such genes include, but are not limited to, B2M, TAP1, CD74, and CIITA. In some embodiments, mammalian cells are modulated to have reduced or abrogated expression in one or more genes associated with natural killer (NK) cell activation, e.g., one more NKG2D ligands, non-limiting examples of which include MICA and MICB.

In some embodiments, mammalian cells are modulated to have reduced or abrogated expression in one or more genes associated with T-cell activation and one or more genes associated with NK cell activation.

Reduction or abrogation of gene expression of any of the genes described herein (e.g., B2M, TAP1, CD74, CIITA, MICA, and/or MICB) may be transient, inducible, or stable. Methods of reducing or abrogating gene expression are known in the art and include, but are not limited to, CRISPR-based interference systems (e.g., using a guide RNA or guide RNAs to target a gene of interest along with a nuclease-inactive Cas enzyme (e.g., dCas9) fused with a transcriptional inactivator), gene knockdown systems (e.g., using siRNAs), gene knockout systems (e.g., via homologous recombination), CRISPR-mediated gene deletion, etc.

### B2M

B2M (beta-2-microglobulin) is also known as IMD43. B2M encodes the protein B2M (i.e., β₂ microglobulin), which is a component MHC class I molecules, and thus has a role in antigen presentation. Reduction or abrogation of B2M expression can prevent assembly of MHC class I molecules at the cell surface. Information regarding B2M and exemplary sequences are available, e.g., in the public database from the National Center for Biotechnology Information under Gene ID 567 (human B2M).

In certain embodiments, the present disclosure provides a modified mammalian cell, e.g., a stem cell, wherein B2M gene expression and/or B2M protein expression is reduced, inhibited, or abrogated. In certain embodiments, the modified cell comprises a genetic modification (e.g., an inactivating mutation) in one or all copies of the B2M gene in the cell genome. In certain embodiments, the genetic modification is made via a genomic editing system as known in the art and/or as described herein. In certain embodiments, a genetic modification of one, two, or all copies of the B2M gene in the cell genome are made or introduced via a CRISPR-Cas genomic editing system.

### TAP1

TAP1 (transporter 1, ATP binding cassette subfamily B member) is also known as, e.g., APT1, PSF1, and ABC17. TAP1 encodes the protein TAP1 (i.e., Transporter associated with antigen processing 1), which is an ABC transporter which is understood to have a role in transporting peptides across the endoplasmic reticulum, where they can be loaded onto MHC class I molecules. Accordingly, reduction or abrogation of TAP1 expression can interfere with antigen presentation. Information regarding TAP1 and exemplary sequences are available, e.g., in the public database from the National Center for Biotechnology Information under Gene ID 6890 (human TAP1).

In certain embodiments, the present disclosure provides a modified mammalian cell, e.g., a stem cell, wherein TAP1 gene expression and/or TAP1 protein expression is reduced, inhibited, or abrogated. In certain embodiments, the modified cell comprises a genetic modification in one or all copies of the TAP1 gene in the cell genome. In certain embodiments, the genetic modification is made via a genomic editing system as known in the art and/or as described herein. In certain embodiments, a genetic modification of one or all copies of the TAP1 gene in the cell genome are made or introduced via a CRISPR-Cas genomic editing system.

### CD74

CD74 (CD74 Molecule) is also known as, e.g., p33, CLIP, and HLADG. CD74 encodes the protein CD74 (i.e., Cluster of Differentiation 74 or HLA-DR antigens-associated invariant chain), which is understood to function as an MHC class II chaperone protein. Accordingly, reduction or abrogation of CD74 expression can interfere with antigen presentation. Information regarding CD74 and exemplary sequences are available, e.g., in the public database from the National Center for Biotechnology Information under Gene ID 972 (human CD74).

In certain embodiments, the present disclosure provides a modified mammalian cell, e.g., a stem cell, wherein CD74 gene expression and/or CD74 protein expression is reduced, inhibited, or abrogated. In certain embodiments, the modified cell comprises a genetic modification in one or all copies of the CD74 gene in the cell genome. In certain embodiments, the genetic modification is made via a genomic editing system as known in the art and/or as described herein. In certain embodiments, a genetic modification of one or all copies of the CD74 gene in the cell genome are made or introduced via a CRISPR-Cas genomic editing system.

### CIITA

CIITA (Class II Major Histocompatibility Complex Transactivator) is also known as C2TA, NLRA, MHC2TA, and CIITAIV. CIITA encodes the protein MHC Class II Transactivator (referred to herein as “CIITA”). CIITA is understood to be a transcriptional co-activator of MHC class II gene transcription. Accordingly, reduction or abrogation of CIITA expression can interfere with antigen presentation. Information regarding CIITA and exemplary sequences are available, e.g., in the public database from the National Center for Biotechnology Information under Gene ID 4261 (human CIITA).

In certain embodiments, the present disclosure provides a modified mammalian cell, e.g., a stem cell, wherein CIITA gene expression and/or CIITA protein expression is reduced, inhibited, or abrogated. In certain embodiments, the modified cell comprises a genetic modification in one or all copies of the CIITA gene in the cell genome. In certain embodiments, the genetic modification is made via a genomic editing system as known in the art and/or as described herein. In certain embodiments, a genetic modification of one or all copies of the CIITA gene in the cell genome are made or introduced via a CRISPR-Cas genomic editing system.

### MICA

MICA (MHC Class I Polypeptide-Related Sequence A) is also known as MIC-A and PERB11.1. MICA encodes the protein MICA (MHC Class I Polypeptide-related sequence A), which is understood to be a ligand of the protein receptor NKG2D, and interaction between MICA and NKG2D can stimulate activation of immune cells expressing the receptor (e.g., NK cells). Accordingly, reduction or abrogation of MICA expression can promote evasion of NK cell recognition. Information regarding MICA and exemplary sequences are available, e.g., in the public database from the National Center for Biotechnology Information under Gene ID 100507436 (human MICA).

In certain embodiments, the present disclosure provides a modified mammalian cell, e.g., a stem cell, wherein MICA gene expression and/or MICA protein expression is reduced, inhibited, or abrogated. In certain embodiments, the modified cell comprises a genetic modification in one or all copies of the MICA gene in the cell genome. In certain embodiments, the genetic modification is made via a genomic editing system as known in the art and/or as described herein. In certain embodiments, a genetic modification of one or all copies of the MICA gene in the cell genome are made or introduced via a CRISPR-Cas genomic editing system.

### MICB

MICB (MHC Class I Polypeptide-Related Sequence B) is also known as PERB1.2. MICB encodes the protein MICB (MHC Class I Polypeptide-related sequence B), which is understood to be a ligand of the protein receptor NKG2D, and interaction between MICB and NKG2D can stimulate activation of immune cells expressing the receptor (e.g., NK cells). Accordingly, reduction or abrogation of MICB expression can promote evasion of NK cell recognition. Information regarding MICB and exemplary sequences are available, e.g., in the public database from the National Center for Biotechnology Information under Gene ID 4277 (human MICB).

In certain embodiments, the present disclosure provides a modified mammalian cell, e.g., a stem cell, wherein MICB gene expression and/or MICB protein expression is reduced, inhibited, or abrogated. In certain embodiments, the modified cell comprises a genetic modification in one or all copies of the MICB gene in the cell genome. In certain embodiments, the genetic modification is made via a genomic editing system as known in the art and/or as described herein. In certain embodiments, a genetic modification of one or all copies of the MICB gene in the cell genome are made or introduced via a CRISPR-Cas genomic editing system.

### III. Modified Cells

The disclosure provides modified mammalian cells comprising one or more modifications that enhance or enable evasion of an immune response (e.g., an immune response of a host organism). In certain embodiments, the one or more modifications result in a substantially non-immunogenic, minimally immunogenic, and/or less immunogenic cell, e.g., as compared to a comparable cell that does not comprise the one or more modifications.

In certain embodiments, the modified mammalian cell of the disclosure is a mouse cell. In certain embodiments, the modified mammalian cell is a primate cell. In certain embodiments, the modified mammalian cell is a non-human primate cell. In certain embodiments, the modified mammalian cell is a human cell.

In certain embodiments, the modified mammalian cell of the disclosure is a stem cell, e.g., an adult stem cell, a pluripotent stem cell, an induced pluripotent stem cell, a multipotent stem cell, and/or an embryonic stem cell. In certain embodiments, the modified mammalian cell is derived from a stem cell, e.g., derived from an adult stem cell, a pluripotent stem cell, an induced pluripotent stem cell, a multipotent stem cell, and/or an embryonic stem cell. In certain embodiments, the modified mammalian cell is the progeny of a stem cell, e.g., the progeny of an adult stem cell, a pluripotent stem cell, an induced pluripotent stem cell, a multipotent stem cell, and/or an embryonic stem cell.

In certain embodiments, the modified mammalian cell of the disclosure is a differentiated cell. The disclosure also provides populations of any such modified differentiated mammalian cells, or differentiated tissues, grafts, or organs comprising, consisting essentially of, or consisting of such modified differentiated mammalian cells. In certain embodiments, the differentiated cell, population of cells, or tissue is generated by differentiating a mammalian stem cell comprising one or more modifications disclosed herein that enable or enhance evasion of an immune response. For example, in certain embodiments, stem cells (e.g., PSCs, e.g., iPSCs or ESCs) obtained or derived from a mammal are modified (e.g., modified ex vivo or in vitro) to comprise one or more of the modifications described herein that enable immune evasion, and, in certain embodiments, said modified stem cells are subsequently differentiated into a particular cell type or tissue. Methods of differentiating stem cells (e.g., pluripotent stem cells such as induced pluripotent stem cells or embryonic stem cells) are known in the art.

In certain embodiments, the modified mammalian cell is a pancreatic beta cell. Methods of differentiating stem cells (e.g., pluripotent stem cells such as induced pluripotent stem cells or embryonic stem cells) into pancreatic beta cells are known in the art, e.g., as described in WO2002086107A2.

In certain embodiments, a modified mammalian cell of the disclosure is an isolated cell.

In certain embodiments, a modified mammalian cell of the disclosure overexpresses MUC15, SIGLEC14, TEX10, and/or GPC4. In certain embodiments, a modified mammalian cell of the disclosure is further characterized by: (a) overexpression of one or more genes associated with inhibition of macrophage phagocytosis; (b) overexpression of one or more genes associated with inhibition of complement activation and deposition; (c) overexpression of one or more genes associated with inhibition of NK cell activation; (d) reduced or abrogated expression of one or more genes associated with T cell activation, (e) reduced or abrogated expression of genes associated with NK cell activation, or (f) any combination of (a), (b), (c), (d), and (e).

In certain embodiments, a modified mammalian cell of the disclosure overexpresses MUC15. In certain embodiments, a modified mammalian cell of the disclosure is further characterized by: (a) overexpression of one or more genes associated with inhibition of macrophage phagocytosis; (b) overexpression of one or more genes associated with inhibition of complement activation and deposition; (c) overexpression of one or more genes associated with inhibition of NK cell activation; (d) reduced or abrogated expression of one or more genes associated with T cell activation, (e) abrogated expression of genes associated with NK cell activation, or (f) any combination of (a), (b), (c), (d), and (e).

In certain embodiments, the modified mammalian cell overexpresses MUC15, and is characterized by overexpression of one or more genes associated with inhibition of macrophage phagocytosis (e.g., CD47 and/or CD24). In certain embodiments, the modified mammalian cell overexpresses MUC15 and CD47. In certain embodiments, the modified mammalian cell overexpresses MUC15 and CD24.

In certain embodiments, the modified mammalian cell overexpresses MUC15, and is characterized by overexpression of one or more genes associated with complement inhibition and deposition (e.g., CD55, CD59, CD46, and/or CR1). In certain embodiments, the modified mammalian cell overexpresses MUC15, CD55, CD59, and CD46.

In certain embodiments, the modified mammalian cell overexpresses MUC15, and is characterized by overexpression of one or more genes associated with inhibition of NK cell activation (e.g., HLA-G and/or HLA-E). In certain embodiments, the modified mammalian cell overexpresses MUC15, HLA-G, and HLA-E.

In certain embodiments, the modified mammalian cell overexpresses MUC15, and is characterized by reduction or abrogation of expression of one or more genes associated with T cell activation (e.g., B2M, TAP1, CD74, and/or CIITA). In certain embodiments, the modified mammalian cell overexpresses MUC15, and expression of B2M, TAP1, CD74, and CIITA in the modified cell is reduced or abrogated.

In certain embodiments, the modified mammalian cell overexpresses MUC15, and is characterized by reduction or abrogation of expression of one or more genes associated with NK cell activation, e.g., one or more genes encoding NKG2D ligands (e.g., MICA and/or MICB). In certain embodiments, the modified mammalian cell overexpresses MUC15, and expression of MICA and MICB in the modified cell is reduced or abrogated.

In certain embodiments, the modified mammalian cell of the disclosure overexpresses MUC15, and is further characterized by: (a) overexpression of one or more genes associated with inhibition of macrophage phagocytosis; (b) overexpression of one or more genes associated with inhibition of complement activation and deposition; (c); overexpression of one or more genes associated with inhibition of NK cell activation; (d) reduced or abrogated expression of one or more genes associated with T cell activation, and (e) reduced or abrogated expression of genes associated with NK cell activation. In certain embodiments, the modified mammalian cell overexpresses MUC15, CD47, CD55, CD59, CD46, HLA-G, and HLA-E. In certain embodiments, the modified mammalian cell overexpresses MUC15, and expression of B2M, TAP1, CD74, CIITA, MICA, and MICB is reduced or abrogated. In certain embodiments, the modified mammalian cell overexpresses MUC15, CD47, CD55, CD59, CD46, HLA-G, and HLA-E, and expression of B2M, TAP1, CD74, CIITA, MICA, and MICB is reduced or abrogated.

In certain embodiments, the modified mammalian cell of the disclosure overexpresses MUC15, and is further characterized by: (a) overexpression of one or more genes associated with inhibition of macrophage phagocytosis; (b) overexpression of one or more genes associated with inhibition of complement activation and deposition; (c) reduced or abrogated expression of one or more genes associated with T cell activation; and (d) reduced or abrogated expression of genes associated with NK cell activation. In certain embodiments, the modified mammalian cell is further characterized by (e) normal expression of HLA-A and HLA-G. In certain embodiments, the modified mammalian cell overexpresses MUC15, CD47, CD55, CD59, and CD46. In certain embodiments, the modified mammalian cell overexpresses MUC15, and expression of B2M, TAP1, CD74, CIITA, MICA, and MICB is reduced or abrogated. In certain embodiments, the modified mammalian cell overexpresses MUC15, CD47, CD55, CD59, and CD46, and expression of B2M, TAP1, CD74, CIITA, MICA, and MICB is reduced or abrogated. In certain further embodiments, expression of HLA-A and HLA-G is normal.

In certain embodiments, a modified mammalian cell of the disclosure overexpresses SIGLEC14. In certain embodiments, a modified mammalian cell of the disclosure is further characterized by: (a) overexpression of one or more genes associated with inhibition of macrophage phagocytosis; (b) overexpression of one or more genes associated with inhibition of complement activation and deposition; (c) overexpression of one or more genes associated with inhibition of NK cell activation; (d) reduced or abrogated expression of one or more genes associated with T cell activation, (e) reduced or abrogated expression of genes associated with NK cell activation, or (f) any combination of (a), (b), (c), (d), and (e).

In certain embodiments, the modified mammalian cell overexpresses SIGLEC14, and is characterized by overexpression of one or more genes associated with inhibition of macrophage phagocytosis (e.g., CD47 and/or CD24). In certain embodiments, the modified mammalian cell overexpresses SIGLEC14 and CD47. In certain embodiments, the modified mammalian cell overexpresses SIGLEC14 and CD24.

In certain embodiments, the modified mammalian cell overexpresses SIGLEC14, and is characterized by overexpression of one or more genes associated with complement inhibition and deposition (e.g., CD55, CD59, CD46, and/or CR1). In certain embodiments, the modified mammalian cell overexpresses SIGLEC14, CD55, CD59, and CD46.

In certain embodiments, the modified mammalian cell overexpresses SIGLEC14, and is characterized by overexpression of one or more genes associated with inhibition of NK cell activation (e.g., HLA-G and/or HLA-E). In certain embodiments, the modified mammalian cell overexpresses SIGLEC14, HLA-G, and HLA-E.

In certain embodiments, the modified mammalian cell overexpresses SIGLEC14, and is characterized by reduced or abrogated expression of one or more genes associated with T cell activation (e.g., B2M, TAP1, CD74, and/or CIITA). In certain embodiments, the modified mammalian cell overexpresses SIGLEC14, and expression of B2M, TAP1, CD74, and CIITA in the modified cell is reduced or abrogated.

In certain embodiments, the modified mammalian cell overexpresses SIGLEC14, and is characterized by reduced or abrogated expression of one or more genes associated with NK cell activation, e.g., one or more genes encoding NKG2D ligands (e.g., MICA and/or MICB). In certain embodiments, the modified mammalian cell overexpresses SIGLEC14, and expression of MICA and MICB in the modified cell is reduced or abrogated.

In certain embodiments, the modified mammalian cell of the disclosure overexpresses SIGLEC14, and is further characterized by: (a) overexpression of one or more genes associated with inhibition of macrophage phagocytosis; (b) overexpression of one or more genes associated with inhibition of complement activation and deposition; (c); overexpression of one or more genes associated with inhibition of NK cell activation; (d) reduced or abrogated expression of one or more genes associated with T cell activation, and (e) reduced or abrogated expression of genes associated with NK cell activation. In certain embodiments, the modified mammalian cell overexpresses SIGLEC14, CD47, CD55, CD59, CD46, HLA-G, and HLA-E. In certain embodiments, the modified mammalian cell overexpresses SIGLEC14, and expression of B2M, TAP1, CD74, CIITA, MICA, and MICB is reduced or abrogated. In certain embodiments, the modified mammalian cell overexpresses SIGLEC14, CD47, CD55, CD59, CD46, HLA-G, and HLA-E, and expression of B2M, TAP1, CD74, CIITA, MICA, and MICB is reduced or abrogated.

In certain embodiments, the modified mammalian cell of the disclosure overexpresses SIGLEC14, and is further characterized by: (a) overexpression of one or more genes associated with inhibition of macrophage phagocytosis; (b) overexpression of one or more genes associated with inhibition of complement activation and deposition; (c) reduced or abrogated expression of one or more genes associated with T cell activation; and (d) reduced or abrogated expression of genes associated with NK cell activation. In certain embodiments, the modified mammalian cell is further characterized by (e) normal expression of HLA-A and HLA-G. In certain embodiments, the modified mammalian cell overexpresses SIGLEC14, CD47, CD55, CD59, and CD46. In certain embodiments, the modified mammalian cell overexpresses SIGLEC14, and expression of B2M, TAP1, CD74, CIITA, MICA, and MICB is reduced or abrogated. In certain embodiments, the modified mammalian cell overexpresses SIGLEC14, CD47, CD55, CD59, and CD46, and expression of B2M, TAP1, CD74, CIITA, MICA, and MICB is reduced or abrogated. In certain further embodiments, expression of HLA-A and HLA-G is normal.

In certain embodiments, a modified mammalian cell of the disclosure overexpresses TEX10. In certain embodiments, a modified mammalian cell of the disclosure is further characterized by: (a) overexpression of one or more genes associated with inhibition of macrophage phagocytosis; (b) overexpression of one or more genes associated with inhibition of complement activation and deposition; (c) overexpression of one or more genes associated with inhibition of NK cell activation; (d) reduced or abrogated expression of one or more genes associated with T cell activation; (e) reduced or abrogated expression of genes associated with NK cell activation; or (f) any combination of (a), (b), (c), (d), and (e).

In certain embodiments, the modified mammalian cell overexpresses TEX10, and is characterized by overexpression of one or more genes associated with inhibition of macrophage phagocytosis (e.g., CD47 and/or CD24). In certain embodiments, the modified mammalian cell overexpresses TEX10 and CD47. In certain embodiments, the modified mammalian cell overexpresses TEX10 and CD24.

In certain embodiments, the modified mammalian cell overexpresses TEX10, and is characterized by overexpression of one or more genes associated with complement inhibition and deposition (e.g., CD55, CD59, CD46, and/or CR1). In certain embodiments, the modified mammalian cell overexpresses TEX10, CD55, CD59, and CD46.

In certain embodiments, the modified mammalian cell overexpresses TEX10, and is characterized by overexpression of one or more genes associated with inhibition of NK cell activation (e.g., HLA-G and/or HLA-E). In certain embodiments, the modified mammalian cell overexpresses TEX10, HLA-G, and HLA-E.

In certain embodiments, the modified mammalian cell overexpresses TEX10, and is characterized by reduced or abrogation of expression of one or more genes associated with T cell activation (e.g., B2M, TAP1, CD74, and/or CIITA). In certain embodiments, the modified mammalian cell overexpresses TEX10, and expression of B2M, TAP1, CD74, and CIITA in the modified cell is reduced or abrogated.

In certain embodiments, the modified mammalian cell overexpresses TEX10, and is characterized by reduced or abrogated of expression of one or more genes associated with NK cell activation, e.g., one or more genes encoding NKG2D ligands (e.g., MICA and/or MICB). In certain embodiments, the modified mammalian cell overexpresses TEX10, and expression of MICA and MICB in the modified cell is reduced or abrogated.

In certain embodiments, the modified mammalian cell of the disclosure overexpresses TEX10, and is further characterized by: (a) overexpression of one or more genes associated with inhibition of macrophage phagocytosis; (b) overexpression of one or more genes associated with inhibition of complement activation and deposition; (c) overexpression of one or more genes associated with inhibition of NK cell activation; (d) reduced or abrogated expression of one or more genes associated with T cell activation, and (e) reduced or abrogated expression of genes associated with NK cell activation. In certain embodiments, the modified mammalian cell overexpresses TEX10, CD47, CD55, CD59, CD46, HLA-G, and HLA-E. In certain embodiments, the modified mammalian cell overexpresses TEX10, and expression of B2M, TAP1, CD74, CIITA, MICA, and MICB is reduced or abrogated. In certain embodiments, the modified mammalian cell overexpresses TEX10, CD47, CD55, CD59, CD46, HLA-G, and HLA-E, and expression of B2M, TAP1, CD74, CIITA, MICA, and MICB is reduced or abrogated.

In certain embodiments, the modified mammalian cell of the disclosure overexpresses TEX10, and is further characterized by: (a) overexpression of one or more genes associated with inhibition of macrophage phagocytosis; (b) overexpression of one or more genes associated with inhibition of complement activation and deposition; (c) reduced or abrogated expression of one or more genes associated with T cell activation; and (d) reduced or abrogated expression of genes associated with NK cell activation. In certain embodiments, the modified mammalian cell is further characterized by (e) normal expression of HLA-A and HLA-G. In certain embodiments, the modified mammalian cell overexpresses TEX10, CD47, CD55, CD59, and CD46. In certain embodiments, the modified mammalian cell overexpresses TEX10, and expression of B2M, TAP1, CD74, CIITA, MICA, and MICB is reduced or abrogated. In certain embodiments, the modified mammalian cell overexpresses TEX10, CD47, CD55, CD59, and CD46, and expression of B2M, TAP1, CD74, CIITA, MICA, and MICB is reduced or abrogated. In certain further embodiments, expression of HLA-A and HLA-G is normal.

In certain embodiments, a modified mammalian cell of the disclosure overexpresses GPC4. In certain embodiments, a modified mammalian cell of the disclosure is further characterized by: (a) overexpression of one or more genes associated with inhibition of macrophage phagocytosis; (b) overexpression of one or more genes associated with inhibition of complement activation and deposition; (c); overexpression of one or more genes associated with inhibition of NK cell activation; (d) reduced or abrogated expression of one or more genes associated with T cell activation; (e) reduced or abrogated expression of genes associated with NK cell activation; or (f) any combination of (a), (b), (c), (d), and (e).

In certain embodiments, the modified mammalian cell overexpresses GPC4, and is characterized by overexpression of one or more genes associated with inhibition of macrophage phagocytosis (e.g., CD47 and/or CD24). In certain embodiments, the modified mammalian cell overexpresses GPC4 and CD47. In certain embodiments, the modified mammalian cell overexpresses GPC4 and CD24.

In certain embodiments, the modified mammalian cell overexpresses GPC4, and is characterized by overexpression of one or more genes associated with complement inhibition and deposition (e.g., CD55, CD59, CD46, and/or CR1). In certain embodiments, the modified mammalian cell overexpresses GPC4, CD55, CD59, and CD46.

In certain embodiments, the modified mammalian cell overexpresses GPC4, and is characterized by overexpression of one or more genes associated with inhibition of NK cell activation (e.g., HLA-G and/or HLA-E). In certain embodiments, the modified mammalian cell overexpresses GPC4, HLA-G, and HLA-E.

In certain embodiments, the modified mammalian cell overexpresses GPC4, and is characterized by reduced or abrogated expression of one or more genes associated with T cell activation (e.g., B2M, TAP1, CD74, and/or CIITA). In certain embodiments, the modified mammalian cell overexpresses GPC4, and expression of B2M, TAP1, CD74, and CIITA in the modified cell is reduced or abrogated.

In certain embodiments, the modified mammalian cell overexpresses GPC4, and is characterized by reduced or abrogated expression of one or more genes associated with NK cell activation, e.g., one or more genes encoding NKG2D ligands (e.g., MICA and/or MICB). In certain embodiments, the modified mammalian cell overexpresses GPC4, and expression of MICA and MICB in the modified cell is reduced or abrogated.

In certain embodiments, the modified mammalian cell of the disclosure overexpresses GPC4, and is further characterized by: (a) overexpression of one or more genes associated with inhibition of macrophage phagocytosis; (b) overexpression of one or more genes associated with inhibition of complement activation and deposition; (c); overexpression of one or more genes associated with inhibition of NK cell activation; (d) reduced or abrogated expression of one or more genes associated with T cell activation, and (e) reduced or abrogated expression of genes associated with NK cell activation. In certain embodiments, the modified mammalian cell overexpresses GPC4, CD47, CD55, CD59, CD46, HLA-G, and HLA-E. In certain embodiments, the modified mammalian cell overexpresses GPC4, and expression of B2M, TAP1, CD74, CIITA, MICA, and MICB is reduced or abrogated. In certain embodiments, the modified mammalian cell overexpresses GPC4, CD47, CD55, CD59, CD46, HLA-G, and HLA-E, and expression of B2M, TAP1, CD74, CIITA, MICA, and MICB is reduced or abrogated.

In certain embodiments, the modified mammalian cell of the disclosure overexpresses GPC4, and is further characterized by: (a) overexpression of one or more genes associated with inhibition of macrophage phagocytosis; (b) overexpression of one or more genes associated with inhibition of complement activation and deposition; (c) reduced or abrogated expression of one or more genes associated with T cell activation; and (d) reduced or abrogated expression of genes associated with NK cell activation. In certain embodiments, the modified mammalian cell is further characterized by (e) normal expression of HLA-A and HLA-G. In certain embodiments, the modified mammalian cell overexpresses GPC4, CD47, CD55, CD59, and CD46. In certain embodiments, the modified mammalian cell overexpresses GPC4, and expression of B2M, TAP1, CD74, CIITA, MICA, and MICB is reduced or abrogated. In certain embodiments, the modified mammalian cell overexpresses GPC4, CD47, CD55, CD59, and CD46, and expression of B2M, TAP1, CD74, CIITA, MICA, and MICB is reduced or abrogated. In certain further embodiments, expression of HLA-A and HLA-G is normal.

In certain embodiments, modifications to cells described in the present disclosure can result in a significant reduction in expression of one or more genes (e.g., B2M, TAP1, CD74, CIITA, MICA, and/or MICB) in the modified cell or in a population of modified cells (e.g., a reduction of at least 5%, at least 10%, at least 20%, at least 30%, at least 40%, at least 50%, at least 60%, at least 70%, at least 80%, at least 90%, at least 95%, or greater reduction). In certain embodiments, expression of a gene (e.g., B2M, TAP1, CD74, CIITA, MICA, or MICB) in the modified cell or population is reduced from about 40% to about 100% (for example, from 40% to 95%, from 40% to 90%, from 40% to 85%, from 40% to 80%, from 40% to 75%, from 40% to 70%, from 40% to 65%, from 40% to 60%, from 40% to 55%, from 40% to 50%, from 40% to 45%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, or from 95% to 100%).

In certain embodiments, modifications to cells described in the present disclosure can result in a significant reduction in activity of a protein expressed by one or more genes (e.g., B2M, TAP1, CD74, CIITA, MICA, and/or MICB) in the modified cell or in a population of modified cells (e.g., a reduction of at least 5%, at least 10%, at least 20%, at least 30%, at least 40%, at least 50%, at least 60%, at least 70%, at least 80%, at least 90%, at least 95%, or greater reduction). In certain embodiments, activity of a protein expressed by a gene (e.g., B2M, TAP1, CD74, CIITA, MICA, and/or MICB) in the modified cell or population of cells is reduced from about 40% to about 100% (for example, from 40% to 95%, from 40% to 90%, from 40% to 85%, from 40% to 80%, from 40% to 75%, from 40% to 70%, from 40% to 65%, from 40% to 60%, from 40% to 55%, from 40% to 50%, from 40% to 45%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, or from 95% to 100%).

In certain embodiments, modifications to cells described in the present disclosure can result in a significant reduction in expression of a polypeptide (e.g., B2M, TAP1, CD74, CIITA, MICA, and/or MICB) in the modified cell or in a population of modified cells (e.g., a reduction of at least 5%, at least 10%, at least 20%, at least 30%, at least 40%, at least 50%, at least 60%, at least 70%, at least 80%, at least 90%, at least 95%, or greater reduction). In certain embodiments, expression of the polypeptide (e.g., B2M, TAP1, CD74, CIITA, MICA, and/or MICB) in the modified cell or population of cells is reduced from about 40% to about 100% (for example, from 40% to 95%, from 40% to 90%, from 40% to 85%, from 40% to 80%, from 40% to 75%, from 40% to 70%, from 40% to 65%, from 40% to 60%, from 40% to 55%, from 40% to 50%, from 40% to 45%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, or from 95% to 100%).

In certain embodiments, modifications to cells described in the present disclosure can result in a significant increase in expression of one or more genes (e.g., MUC15, SIGLEC14, TEX10, GPC4, CD47, CD55, CD59, CD46, HLA-G, and/or HLA-E) in the modified cell or population of modified cells (e.g., an increase of at least 1.1-fold, at least 1.5-fold, at least 2-fold, at least 5-fold, at least 10-fold, at least 25-fold, at least 50-fold, at least 100-fold, at least 500-fold, or at least 1000-fold). In certain embodiments, expression of a gene (e.g., MUC15, SIGLEC14, TEX10, GPC4, CR1, CD24, CD47, CD55, CD59, CD46, HLA-G, and/or HLA-E) in the modified cell or population of cells is increased by 1.1-fold to 1000-fold, by 1.1-fold to 500-fold, by 1.1-fold to 100-fold, by 1.1-fold to 50-fold, by 1.1-fold to 25-fold, by 1.1-fold to 10-fold, by 1.1-fold to 5-fold, by 1.1-fold to 2-fold, by 1.1-fold to 1.5-fold, by 1.5-fold to 1000-fold, by 1.5-fold to 500-fold, by 1.5-fold to 100-fold, by 1.5-fold to 50-fold, by 1.5-fold to 25-fold, by 1.5-fold to 10-fold, by 1.5-fold to 5-fold, by 1.5-fold to 2-fold, by 2-fold to 1000-fold, by 2-fold to 500-fold, by 2-fold to 100-fold, by 2-fold to 50-fold, by 2-fold to 25-fold, by 2-fold to 10-fold, by 2-fold to 5-fold, by 5-fold to 1000-fold, by 5-fold to 500-fold, by 5-fold to 100-fold, by 5-fold to 50-fold, by 5-fold to 25-fold, by 5-fold to 10-fold, by 10-fold to 1000-fold, by 10-fold to 500-fold, by 10-fold to 100-fold, by 10-fold to 50-fold, by 10-fold to 25-fold, by 25-fold to 1000-fold, by 25-fold to 500-fold, by 25-fold to 100-fold, by 25-fold to 50-fold, by 50-fold to 1000-fold, by 50-fold to 500-fold, by 50-fold to 100-fold, by 100-fold to 1000-fold, by 100-fold to 500-fold, or by 500-fold to 1000-fold.

In certain embodiments, modifications to cells described in the present disclosure can result in a significant increase in expression of a polypeptide (e.g., MUC15, Siglec-14, Tex10, Glypican-4, CD47, CR1, CD24, CD55, CD59, CD46, HLA-G, and/or HLA-E) in the modified cell or population of modified cells (e.g., an increase of at least 1.1-fold, at least 1.5-fold, at least 2-fold, at least 5-fold, at least 10-fold, at least 25-fold, at least 50-fold, at least 100-fold, at least 500-fold, or at least 1000-fold). In certain embodiments, expression of a polypeptide (e.g., MUC15, Siglec-14, Tex10, Glypican-4, CR1, CD24, CD47, CD55, CD59, CD46, HLA-G, and/or HLA-E) in the modified cell or population of cells is increased by 1.1-fold to 1000-fold, by 1.1-fold to 500-fold, by 1.1-fold to 100-fold, by 1.1-fold to 50-fold, by 1.1-fold to 25-fold, by 1.1-fold to 10-fold, by 1.1-fold to 5-fold, by 1.1-fold to 2-fold, by 1.1-fold to 1.5-fold, by 1.5-fold to 1000-fold, by 1.5-fold to 500-fold, by 1.5-fold to 100-fold, by 1.5-fold to 50-fold, by 1.5-fold to 25-fold, by 1.5-fold to 10-fold, by 1.5-fold to 5-fold, by 1.5-fold to 2-fold, by 2-fold to 1000-fold, by 2-fold to 500-fold, by 2-fold to 100-fold, by 2-fold to 50-fold, by 2-fold to 25-fold, by 2-fold to 10-fold, by 2-fold to 5-fold, by 5-fold to 1000-fold, by 5-fold to 500-fold, by 5-fold to 100-fold, by 5-fold to 50-fold, by 5-fold to 25-fold, by 5-fold to 10-fold, by 10-fold to 1000-fold, by 10-fold to 500-fold, by 10-fold to 100-fold, by 10-fold to 50-fold, by 10-fold to 25-fold, by 25-fold to 1000-fold, by 25-fold to 500-fold, by 25-fold to 100-fold, by 25-fold to 50-fold, by 50-fold to 1000-fold, by 50-fold to 500-fold, by 50-fold to 100-fold, by 100-fold to 1000-fold, by 100-fold to 500-fold, or by 500-fold to 1000-fold.

In certain embodiments, modifications to cells described in the present disclosure can result in a significant increase in in activity of a protein expressed by one or more genes (e.g., MUC15, Siglec-14, Tex10, Glypican-4, CD47, CR1, CD24, CD55, CD59, CD46, HLA-G, and/or HLA-E) in the modified cell or population of modified cells (e.g., an increase of at least 1.1-fold, at least 1.5-fold, at least 2-fold, at least 5-fold, at least 10-fold, at least 25-fold, at least 50-fold, at least 100-fold, at least 500-fold, or at least 1000-fold). In certain embodiments, activity of a protein (e.g., MUC15, Siglec-14, Tex10, Glypican-4, CR1, CD24, CD47, CD55, CD59, CD46, HLA-G, and/or HLA-E) in the modified cell or population of cells is increased by 1.1-fold to 1000-fold, by 1.1-fold to 500-fold, by 1.1-fold to 100-fold, by 1.1-fold to 50-fold, by 1.1-fold to 25-fold, by 1.1-fold to 10-fold, by 1.1-fold to 5-fold, by 1.1-fold to 2-fold, by 1.1-fold to 1.5-fold, by 1.5-fold to 1000-fold, by 1.5-fold to 500-fold, by 1.5-fold to 100-fold, by 1.5-fold to 50-fold, by 1.5-fold to 25-fold, by 1.5-fold to 10-fold, by 1.5-fold to 5-fold, by 1.5-fold to 2-fold, by 2-fold to 1000-fold, by 2-fold to 500-fold, by 2-fold to 100-fold, by 2-fold to 50-fold, by 2-fold to 25-fold, by 2-fold to 10-fold, by 2-fold to 5-fold, by 5-fold to 1000-fold, by 5-fold to 500-fold, by 5-fold to 100-fold, by 5-fold to 50-fold, by 5-fold to 25-fold, by 5-fold to 10-fold, by 10-fold to 1000-fold, by 10-fold to 500-fold, by 10-fold to 100-fold, by 10-fold to 50-fold, by 10-fold to 25-fold, by 25-fold to 1000-fold, by 25-fold to 500-fold, by 25-fold to 100-fold, by 25-fold to 50-fold, by 50-fold to 1000-fold, by 50-fold to 500-fold, by 50-fold to 100-fold, by 100-fold to 1000-fold, by 100-fold to 500-fold, or by 500-fold to 1000-fold.

In certain embodiments, reduction or abrogation in expression and/or activity of one or more genes (e.g., B2M, TAP1, CD74, CIITA, MICA, or MICB) or protein products thereof in a modified cell, population of cells, or modified cell line is maintained for at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 4 months, at least 5 months, at least 6 months, at least 8 months, at least 10 months, at least 1 year, at least 2 years, at least 5 years, or at least 10 years. In certain embodiments, reduction in expression and/or activity of one or more genes (e.g., B2M, TAP1, CD74, CIITA, MICA, or MICB) in a modified cell, population of cells, or cell line is intended to be maintained indefinitely or permanently, e.g., through the use of a gene disruption or a partial or complete gene deletion.

In certain embodiments, an increase in expression and/or activity of one or more genes (e.g., MUC15, SIGLEC14, TEX10, GPC4, CR1, CD24, CD47, CD55, CD59, CD46, HLA-G, and/or HLA-E) or protein products thereof in a modified cell, population of cells, or modified cell line is maintained for at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 4 months, at least 5 months, at least 6 months, at least 8 months, at least 10 months, at least 1 year, at least 2 years, at least 5 years, or at least 10 years. In certain embodiments, an increase in expression and/or activity of one or more genes (e.g., B2M, TAP1, CD74, CIITA, MICA, or MICB) in a modified cell, population of cells, or cell line is intended to be maintained indefinitely or permanently, e.g., through the introduction of heterologous nucleic acids to the host genome.

Gene expression or protein expression may be measured using any suitable technique known in the art, including, but not limited to, reverse transcriptase polymerase chain reactions (RT-PCT, e.g., quantitative RT-PCR), western blot, flow cytometry, FACS, ELISA, immunoassays, etc.

### V. Methods of Modifying Cells

Also provided herein are compositions and methods for modifying cells as described herein.

In general, cells can be genetically modified through the introduction of mutations (e.g., insertions or deletions) to the genome. Additionally or alternatively, cells can be genetically modified through introduction (i.e., delivery) of one or more polynucleotides described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be modified or engineered.

### Viral-Mediated Delivery

Viral expression vectors or systems can be used to modify cells. In general, viral expression vectors can be used to modify a cell through introducing (i.e., delivering) a nucleic acid into a host cell (e.g., a mammalian stem cell). For example, a viral vector-based delivery platform can modify a cell through introducing any of the heterologous nucleic acids described herein. Alternatively or additionally, a viral vector can also be used to reduce or inhibit expression of one or more genes of interest (e.g., through disruption of a native gene or associated regulatory sequence, or through expression of a negative regulatory element). A viral expression vector can be a nucleic acid, and as such, an engineered, heterologous nucleic acid of the disclosure can also encompass an engineered viral expression vector. Such vectors can also be referred to as recombinant viruses or engineered viruses.

A viral expression vector can encode more than one heterologous nucleic acid, gene, or transgene within the same nucleic acid. For example, a viral expression vector, e.g., a recombinant virus or an engineered virus, can encode one or more genes or proteins of interest described herein including, but not limited to, MUC15, SIGLEC14, TEX10, GPC4, CD46, CD59, CD55, CD47, HLA-G, HLA-E, CD24, CR1 and combinations thereof. A viral expression vector can encode one or more genes in addition to the one or more genes or proteins of interest, such as viral genes needed for viral infectivity and/or viral production (e.g., capsid proteins, envelope proteins, viral polymerases, viral transcriptases, etc.), referred to as cis-acting elements or genes.

A viral expression vector system can comprise more than one viral vector, such as separate viral vectors encoding the one or more genes or proteins of interest. For example, a helper-dependent viral vector-based delivery platform can provide additional genes needed for viral infectivity and/or viral production on one or more additional separate vectors in addition to the vector encoding the one or more genes or proteins of interest. One viral vector can deliver more than one heterologous nucleic acids, such as one vector that delivers heterologous nucleic acids that are configured to encode or express two or more genes/proteins of interest. More than one viral vector can deliver more than one heterologous nucleic acids, such as more than one vector that delivers one or more heterologous nucleic acid configured to encode or express one or more genes or proteins of interest. The number of viral vectors used can depend on the packaging capacity of the above-mentioned viral vector-based vaccine platforms, and one skilled in the art can select the appropriate number of viral vectors.

In general, any of the viral vector-based systems can be used e.g., for in vivo delivery of the heterologous nucleic acids encoding one or more genes or proteins of interest. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., a modified cell) can express the one or more genes of interest encoded by the engineered viral vector. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of heterologous nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., a heterologous nucleic acid) encoding one or more genes or proteins of interest. The transgenes encoding the one or more genes or proteins of interest can be configured to express the one or more genes or proteins of interest.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more heterologous nucleic acids (e.g., nucleic acids encoding the one or more genes or proteins of interest) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immunodeficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that can transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with heterologous nucleic acids (e.g., any of the heterologous nucleic acids described herein). AAV systems can be used for the in vitro expression of genes or proteins, or used for in vivo and ex vivo gene therapy procedures, e.g., for in vivo delivery of the heterologous nucleic acids encoding one or more genes or proteins of interest (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the heterologous nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Where heterologous nucleic acids described herein are incorporated into a viral vector comprising an RNA genome, it is contemplated that any thymine in the heterologous nucleic acid sequence may be replaced with a uracil, as appropriate.

### Lipid Structure Delivery Systems

Heterologous nucleic acids of the present disclosure (e.g., any of the heterologous nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the heterologous nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., a heterologous nucleic acid, such as any of the heterologous nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers, and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the heterologous nucleic acids encoding genes or proteins of interest described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the heterologous nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the heterologous nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the heterologous nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the heterologous nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethylene glycol (PEG) conjugates (PEGylated lipids), waxes, oils, glycerides, and fat-soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

### Nanoparticle Delivery

Nanomaterials can be used to deliver heterologous nucleic acids (e.g., any of the heterologous nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

### Genomic Editing Systems

A genomic editing system can be used to engineer a host genome, e.g., to modify a target nucleic acid in a cell. Certain genomic editing systems may be used to introduce mutations into a cell genome (e.g., by introducing one or more substitutions, insertions, or deletions, into one or more copies of a target gene or an associated regulatory region, and/or by partially or completely deleting one or more copies of a gene). Such mutations may result in the abrogation of gene or protein expression. Certain genomic editing systems may also be used to introduce heterologous nucleic acids into the genome of a modified cell. The introduction of heterologous nucleic acids into the genome can be used to disrupt gene or protein expression, e.g., via the introduction of a nucleic acid that disrupts the transcription, translation, or function of a target gene. Additionally or alternatively, the introduction of heterologous DNA via a genomic editing system may be used to introduce a nucleic acid encoding one or more genes or proteins of interest, thereby resulting in the overexpression of said gene or protein. The introduction of heterologous regulatory elements into certain genomic sites (or, conversely, the disruption of native regulatory elements) may likewise be used to alter expression of a gene or protein (e.g., by abrogating expression or by increasing expression). Genomic editing systems include, but are not limited to, transposon systems and nuclease genomic editing systems (e.g., rare-cutting endonucleases, e.g., CRISPR-Cas systems).

A transposon system can be used to integrate a heterologous nucleic acid, such as a heterologous nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., a heterologous nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode a heterologous nucleic acid, such as a heterologous nucleic acid of the present disclosure. Without wishing to be bound by theory, in general, the nuclease-mediated gene editing systems take advantage of a cell's natural DNA repair mechanisms. Briefly, following an insult to genomic DNA (typically a double-stranded break), the cell can resolve the insult either by error-prone non-homologous end-joining (NHEJ), or through homologous recombination (HR) repair pathway. NHEJ is error-prone, and can be useful for introducing mutations, e.g., insertions or deletions, which can reduce or abrogate expression and/or activity of the target gene or protein. A cell can also resolve the insult by using another DNA source that has identical, or substantially identical, sequences at both its 5′ and 3′ ends as a template during DNA synthesis to repair the lesion. In a natural context, HDR can use the other chromosome present in a cell as a template. In genomic editing systems, exogenous polynucleotides can be introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the heterologous nucleic acids described herein, such as any of the heterologous nucleic acids encoding one or more genes or proteins of interest).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to introduce mutations (e.g., insertions or deletions) to a target nucleic acid. Additionally or alternatively, a CRISPR-mediated gene editing system can be used to engineer a host genome to encode a heterologous nucleic acid, such as a heterologous nucleic acid encoding one or more genes or proteins of interest described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). The design of particular gRNAs (e.g., sgRNAs) is within the capabilities of those skilled in the art. While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a stem cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofector/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The modified cells described herein can be modified using non-viral methods, e.g., the nuclease and/or CRISPR mediated gene editing systems described herein can be delivered to a cell using non-viral methods. The modified cells described herein can be modified using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease FokI. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other CRISPR-Cas Gene Expression Modulation Systems

A variation of the CRISPR-Cas system comprising a nuclease with reduced or no catalytic activity may also be used to modulate gene expression in a cell.

In embodiments described herein, the Cas nuclease in a CRISPR-Cas system (e.g., Cas9) can be mutated to render the enzyme catalytically inactive, or less catalytically active than the wild type Cas9. dCas9 refers to a Cas9 protein, or functional fragment thereof, that has decreased nuclease activity relative to a Cas9 protein found in nature. In certain embodiments, the dCas9 is a mutant form of Cas9 whose endonuclease activity is reduced or removed through mutations (e.g., point mutations) in its endonuclease domains. Cas9 ordinarily has 2 endonuclease domains called the RuvC and HNH domains. In certain embodiments, the point mutations D10A and H840A deactivates the normal Cas9 endonuclease activity.

dCas9 may be used in conjunction with gRNAs (e.g., an sgRNA) to target the catalytically dead enzyme to specific genes. As described above, the sgRNA provides targeted nucleic acid binding activity to Cas9. Although dCas9 lacks endonuclease activity relative to Cas9, it is still capable of binding to its guide RNA (e.g., sgRNA). Through the targeting capability conferred by the guide RNA, the dCas9 enzyme can interact with the targeted DNA strand. dCas9 can then attenuate or block transcription of the targeted gene, thereby reducing or abrogating expression of the targeted gene. This method is known in the art as “CRISPR inhibition” or “CRISPRi”.

In embodiments, the ability of dCas9 to bind DNA can also be exploited for gene activation, e.g., by modifying dCas9 to be fused or attached to transcriptional activators, or by modifying dCas9 to recruit transcriptional activators. In certain embodiments, a transcriptional activator can be, e.g., fused to the N-terminus or C-terminus of dCas9. This method is known int the art as “CRISPR activation” or “CRISPRa”. In certain embodiments, dCas9 is fused to the activation domain VP64. Additional suitable activation domains are described in, e.g., Casas-Mollano et al., “CRISPR-Cas Activators for Engineering Gene Expression in Higher Eukaryotes”, CRISPR J. (2020) 3(5): 350-364.

### Other Delivery Systems

Various additional means to introduce heterologous nucleic acids (e.g., any of the heterologous nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the heterologous nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing heterologous nucleic acids (e.g., any of the heterologous nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60), each herein incorporated by reference for all purposes.

Double Stranded RNA (dsRNA) Molecules for Modulating Expression

In certain embodiments, double-stranded RNA (dsRNA) molecules may be used to modulate expression of one or more genes in a cell or cell line described herein. dsRNA molecules can be designed to antagonize one or more genes by sequence homology-based targeting of the corresponding RNA sequence. Such dsRNAs can be, but are not limited to, small interfering RNAs (siRNAs), small hairpin RNAs (shRNAs), or micro-RNAs (miRNAs). The sequence of such dsRNAs will comprise a complementary portion of the mRNA encoding the one or more genes to be modulated. This portion can be 100% complementary to the target portion within the mRNA, but lower levels of complementarity (e.g., 90% or more or 95% or more) can also be used. Typically, the percent complementarity is determined over a length of contiguous nucleic acid residues. A dsRNA molecule of the disclosure may, for example, have at least 80% complementarity to the target portion within the mRNA measured over at least 10, at least 20, at least 30, at least 40, at least 50, at least 60, at least 70, at least 80, at least 90, or more nucleic acid residues. In some instances, dsRNA molecule has at least 80% complementarity to the target portion of mRNA over the entire length of the dsRNA molecule.

Another gene targeting reagent that uses RNA interference (RNAi) pathways is small hairpin RNA, also referred to as shRNA. shRNAs delivered to cells via, e.g., expression constructs (e.g., plasmids, lentiviruses) have the ability to provide long term reduction of gene expression in a constitutive or regulated manner, depending upon the type of promoter employed. In one embodiment, the genome of a lentiviral particle is modified to include one or more shRNA expression cassettes that target a gene (or genes) of interest. Such lentiviruses can infect a cell, stably integrate their viral genome into the host genome, and express a shRNA in a constitutive, regulated, or (in the case where multiple shRNA are being expressed) constitutive and regulated fashion. Thus, in some embodiments shRNA can be designed to target individual variants of a single gene or multiple closely related gene family members. Individual shRNA can modulate collections of targets having similar or redundant functions or sequence motifs. The skilled person will recognize that lentiviral constructs can also incorporate cloned DNA, or ORF expression constructs.

In certain embodiments described herein, gene targeting reagents including small interfering RNAs (siRNA) as well as microRNAs (miRNA) can be used to modulate gene function. siRNAs and miRNAs can incorporate a wide range of chemical modifications, levels of complementarity to the target transcript of interest, and designs (see U.S. Pat. No. 8,188,060) to enhance stability, cellular delivery, specificity, and functionality. In addition, such reagents can be designed to target diverse regions of a gene (including the 5′ UTR, the open reading frame, the 3′ UTR of the mRNA), or (in some cases) the promoter/enhancer regions of the genomic DNA encoding the gene of interest. Gene modulation (e.g., reduction of gene expression, knockdown) can be achieved by introducing (into a cell) a single siRNA or miRNA or multiple siRNAs or pools of miRNAs targeting different regions of the same mRNA transcript. Synthetic siRNA/miRNA delivery can be achieved by any number of methods including but not limited to 1) self-delivery, 2) lipid-mediated delivery, 3) electroporation, or 4) vector/plasmid-based expression systems. An introduced RNA molecule may be referred to as an exogenous nucleotide sequence or polynucleotide. In some embodiments, siRNA can be designed to target individual variants of a single gene or multiple closely related gene family members.

### Modulation of Expression at the Protein Level

In another embodiment, modulation of expression and/or activity of a gene or protein of interest takes place at the protein (e.g., polypeptide) level. By way of example, reduction of gene function at the protein level can be achieved by methods including, but not limited to, targeting the protein with a small molecule, a peptide, an aptamer, destabilizing domains, or other methods that can e.g., reduce or inhibit the activity or enhance the rate of degradation of a gene product. Alternatively, the expressed protein may be modified to reduce or eliminate biological activity through site-directed mutagenesis and/or the incorporation of missense or nonsense mutations. In some embodiments, a small molecule that binds, e.g., an active site and inhibits the function of a target protein can be added to, e.g., the cell culture media and thereby be introduced into a packaging and/or producer cell. Alternatively, target protein function can be modulated by introducing, e.g., a peptide into a cell (e.g., a packaging and/or producer cell) that for instance prevents protein-protein interactions (see Shangary et. al., (2009) Annual Review of Pharmacology and Toxicology 49:223). Such peptides can be introduced into a cell (e.g., a packaging and/or producer cell) by, for example, transfection or electroporation, or via an expression construct. Alternatively, peptides can be introduced into a cell (e.g., a packaging and/or producer cell) by adding (e.g., through conjugation) one or more moieties that facilitate cellular delivery, or supercharging molecules to enhance self-delivery. Techniques for expressing a peptide include, but are not limited to, fusion of the peptide to a scaffold, or attachment of a signal sequence, to stabilize or direct the peptide to a position or compartment of interest, respectively.

### VI. Methods of Assessing Immune Evasion

The ability of a cell or population of cells (such as a modified mammalian cell disclosed herein, or a cell or tissue differentiated therefrom) to evade immune rejection (e.g., in a host organism) can be tested using any of a variety of methods known in the art or adapted therefrom. For example, methods for testing an immune response to a cell or tissue can be adapted for use as methods to assess immune evasion.

Both in vitro and in vivo assays for immune evasion are available. Non-limiting examples of in vitro assays for immune evasion include macrophage phagocytosis assays, complement deposition assays, and natural killer (NK) cell assays. A non-limiting example of an in vivo assay for immune evasion is a xenotransplantation assay in which cells are transplanted into immune competent mice whose immune systems are typically capable of rejecting foreign cells and tissues (e.g., as exemplified in Example 5 hereinbelow). The ability of transplanted cells and tissues to proliferate in such mice may indicate an ability to evade an immune response. In some assays, the ability of transplanted cells or tissues to form teratomas in certain immune competent mice indicates the ability to evade immune rejection.

### VII. Methods of Treatment and Formulations

The disclosure also provides a method of treating a disease or disorder in a subject in need thereof. In some aspects, the method comprises administering to the subject a modified mammalian cell of the present disclosure. In certain embodiments, the modified cell is a stem cell (e.g., an induced pluripotent stem cell or an embryonic stem cell). In certain embodiments, the modified mammalian cell is derived from a stem cell, e.g., derived from an adult stem cell, a pluripotent stem cell, an induced pluripotent stem cell, a multipotent stem cell, and/or an embryonic stem cell. In certain embodiments, the modified mammalian cell is the progeny of a stem cell, e.g., the progeny of an adult stem cell, a pluripotent stem cell, an induced pluripotent stem cell, a multipotent stem cell, and/or an embryonic stem cell. In certain embodiments, the modified cell is a differentiated cell, e.g., a cell that is the progeny of a mammalian stem cell that was modified as described herein.

In certain embodiments, a method comprises administering to the subject a modified cell that is allogeneic to the subject. In certain embodiments, the modifications to the mammalian cell allow the administered cell to evade an immune response of the subject.

In certain embodiments, the method comprises administering progeny of a genetically modified stem cell of the disclosure, e.g., a partially or terminally differentiated cell or tissue. In certain embodiments, the progeny is a progenitor cell generated from a modified stem cell of the present disclosure. A progenitor cell is a biological cell that, like a stem cell, has a tendency to differentiate into a specific type of cell, but is already more specific than a SC and is pushed to differentiate into its “target” cell.

In certain embodiments of a therapeutic method of the disclosure, the disease or disorder to be treated is type 1 diabetes (T1D).

In certain embodiments of a therapeutic method of the disclosure, the subject is a mammal. In certain embodiments, the subject is a human. In certain embodiments, the subject has T1D, has been diagnosed with T1D, or is at risk for T1D.

In certain embodiments of a therapeutic method of the disclosure, the modified cell to be administered to the subject in need thereof is a pancreatic islet cell. In certain embodiments, the modified cell is a pancreatic islet progenitor cell, an immature pancreatic islet cell, or a mature pancreatic islet cell. In certain embodiments, the modified cell is a pancreatic beta cell. In certain embodiments, the modified cell is derived from (e.g., is the progeny of) a modified stem cell of the disclosure. The modified stem cells of the disclosure may be used to generate pancreatic islet cells using any appropriate differentiation technique known in the art. Exemplary beta cell markers or beta cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Soxl7, and FoxA2. In certain embodiments, the modified pancreatic beta cells of the disclosure produce insulin, e.g., in response to glucose.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4.sup.th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

For therapeutic use, the agents and compositions described herein preferably are combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see, e.g., Adeboye Adejare, Remington: The Science and Practice of Pharmacy (23d ed. 2020). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining, or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants see, e.g., Adeboye Adejare, Remington: The Science and Practice of Pharmacy (23d ed. 2020).

The formulation should suit the mode of administration. The agents of use with the present disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal.

Agents or compositions described herein can also be used in combination with other therapeutic modalities. Thus, in addition to the therapies described herein, one may also provide to the subject other therapies known to be efficacious for treatment of the disease, disorder, or condition.

### EXAMPLES

The following Examples are merely illustrative and are not intended to limit the scope or content of the invention in any way.

### Example 1. Generation of Modified Stem Cells Deficient for HLA and MICA/B

Genetically modified H1 human embryonic stem cells (hESCs) were generated through CRISPR-based targeted mutations and lentiviral expression. A Cas9-expression construct and different gRNA-encoding vectors, targeting the β2m, TAP1, and CD74 genes, were co-transfected into H1 hESCs. Individual colonies were manually picked and subjected to MiSeq™ analysis of targeted genes. Candidate clones were then plated at 1 cell/well by fluorescence-activated cell sorting (FACS), and MiSeq™ analysis was performed to confirm the mutations and lack of mosaicism. One identified clone carried inactivating mutations in both alleles of β2m and TAP1 and in one allele of CD74, which was abrogated with interferon-gamma-induced HLA-I expression and confirmed a normal karyotype. This clone was subsequently re-targeted using CRISPR to ablate the remaining allele of CD74 and both alleles of CIITA, a transcription factor required for expression of HLA-II. As such, an HLA-deficient line was obtained.

To evade potential NK cell-mediated cytolysis of the HLA-deficient line, NKG2D ligands MICA and MICB were targeted via CRISPR/Cas9. Sequence analysis of the nucleofected clones revealed one clone carrying frameshift mutations in all 4 alleles of MICA and MICB. The clone was then validated for normal karyotype and lack of MICA/MICB expression. This HLA-, MICA/B-deficient hESC line is also referred to as “KG-modified” or “BM-KO” herein (see TABLE 1).

Thus, the KG-modified cell line generated in this Example is deficient for β2m, TAP1, CD74, CIITA, MICA, and MICB.

TABLE 1
Cell line information

| Cell Line | KO-Modified Genes | Overexpressed Gene(s) |
|---|---|---|
| HM-KO | β2m, TAP1, CD74, | |
| | CIITA, MICA, MICB | |
| HM-KO-Lenti | β2m, TAP1, CD74, | CD46/Crry, CD55, CD59, HLA-G/K^{b} |
| | CIITA, MICA, MICB | single chain trimers, CD47 |
| HM-KO-6KI | β2m, TAP1, CD74, | CD46/Crry, CD55, CD59, HLA-G/K^{b} |
| | CIITA, MICA, MICB | single chain trimers, CD47, HLA-E/Qa1 |
| | | single chain trimers ¹ |
| HM-KO-Complement | β2m, TAP1, CD74, | CD46/Crry, CD55, CD59, CD47 |
| and Phagocytosis | CIITA, MICA, MICB | |
| (noNK) | | |
| HM-KO-Complement | β2m, TAP1, CD74, | CD46/Crry, CD55, CD59, HLA-G/K^{b}- |
| and NK | CIITA, MICA, MICB | single chain trimers, HLA-E/Qa1 single |
| (noPhagocytosis) | | chain trimers |
| HM-KO-NK and | B2m, TAP1, CD74, | HLA-G/K^{b} single chain trimers, CD47, |
| Phagocytosis | CIITA, MICA, MICB | HLA-E/Qa1 single chain trimers |
| (noComplement) | | |
| HM-KO-Phagocytosis | β2m, TAP1, CD74, | CD47 |
| | CIITA, MICA, MICB | |
| HM-KO-Complement | β2m, TAP1, CD74, | CD46/Crry, CD55, CD59 |
| | CIITA, MICA, MICB | |

¹ HLA-E (or mouse homolog Qa1) single chain trimers may engage inhibitory receptors on NK cells.

### Example 2. Modified Stem Cells with Increased Expression of Certain Genes May Evade Immune Rejection

To evaluate factors that may mediate graft rejection, the HM-KO hESC line was modified to express genes predicted to alleviate mechanisms of graft rejection, such as those mediated by T cells, B cells, NK cells, macrophage phagocytosis, and complement deposition.

Individual lentiviral constructs, each encoding a GFP marker and one of Crry (mouse homolog of CD46), CD55, CD59, and K^{b} (mouse homolog of HLA-G) single chain trimer were generated. CD46, CD55, and CD59 may inhibit complement activation and deposition; HLA-G single chain trimer may engage inhibitory receptors on NK cells. HM-KO cells were transduced with the lentiviral constructs, such that each of Crry, CD59, CD55, and K^{b} single chain trimer genes are present in ˜30% cells (i.e., about 30% cells were infected with lentiviruses carrying a given gene), yielding a cell mixture.

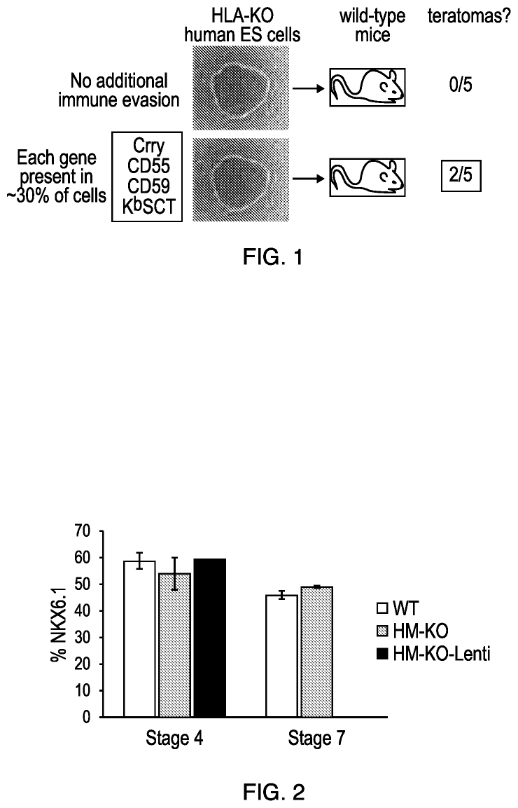

The cell mixture was transplanted into immunocompetent C57B16/J mice, and the growth of teratomas was monitored. As a control, HM-KO cells were transplanted, and the teratoma growth was monitored (FIG. 1). During the monitoring period, several mice transplanted with the cell mixture with increased expression of CD46, CD55, CD59, and K^{b} single chain trimer showed transient teratomas growth. After 8 weeks, 2 out of 5 mice transplanted with the cell mixture had clear teratomas. No teratoma growth was detected at any timepoint in mice transplanted with HM-KO.

Further, the cell mixture was sorted using FACS for cells that 100% express Crry, CD59, and K^{b} single chain trimer, approximately 50% of which also expressed CD55. Subsequently, the sorted cells were additionally transduced with CD47-encoding lentiviral construct, and about 20% of the resulting cells expressed CD47. CD47 may prevent phagocytosis and antigen presentation to T cells by macrophages. The yielding mixture of cells with increased expression of Crry, CD59, K^{b} single chain trimer, CD55, and CD47 is also referred to as “HM-KO-Lenti” herein. The HM-KO-Lenti cells were then differentiated into pancreatic β cells using automated procedures.

These experiments and data suggested that expressing certain genes do not affect the differentiation efficiency of stem cells (FIG. 2) but allow stem cells to avoid rejection and grow in xenogeneic recipients.

### Example 3. Modified Stem Cells with Increased Expression of Certain Genes May Evade Immune Rejection

To evaluate factors that may mediate graft rejection, the HM-KO hESC line was modified to express genes predicted to alleviate mechanisms of graft rejection, such as those mediated by T cells, B cells, NK cells, macrophage phagocytosis, and complement deposition.

100 stem cell derived pseudo-islets derived from HM-KO and HM-KO-Lenti cells were transplanted subcutaneously into 6-8-week-old female mice (n=4). As a positive control, 100 pseudo-islets were also transplanted into immunodeficient NSG mice (n=2).

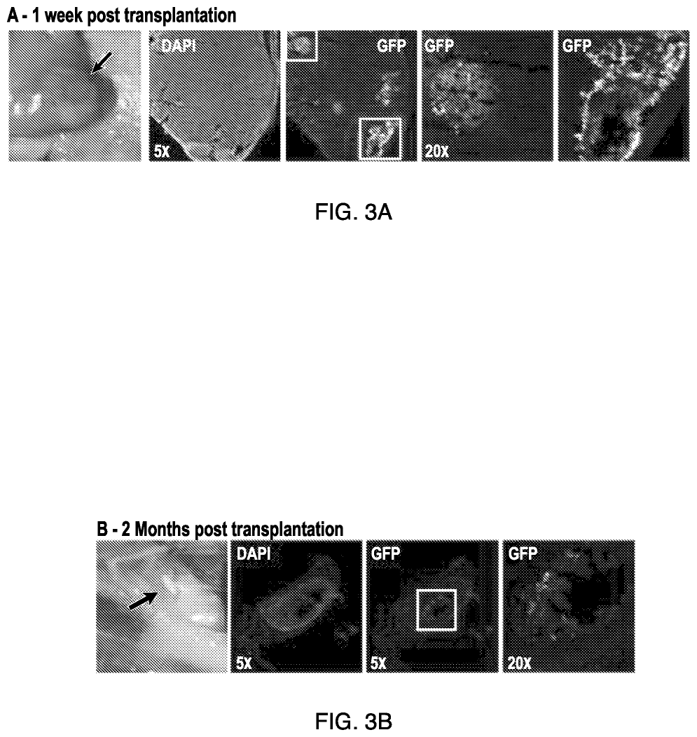

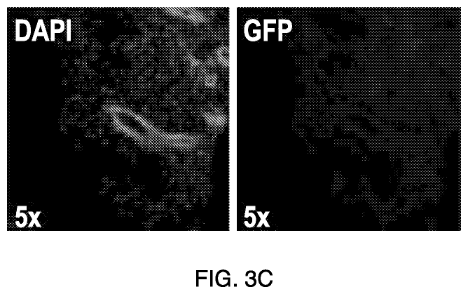

One week after transplantation, one mouse per experimental group was sacrificed to evaluate the survival of the pseudo-islets. In the mouse transplanted with HM-KO-Lenti derived pseudo-islets, grafts were visible at the transplantation site (FIG. 3A), and anti-GFP antibody stain confirmed their human origin through immunohistochemistry (IHC). Two months after transplantation, the remaining mice were sacrificed. None of the mice transplanted with HM-KO cells had visible grafts; one of the three mice transplanted with HM-KO-Lenti cells had a small but clearly defined graft (FIG. 3B). The human stem cell origin of this tissue was also confirmed with GFP IHC (FIG. 3B). FIG. 3C shows staining of a neighboring mammary gland that is negative for GFP.

These data suggest that expressing certain genes allows stem cell-derived tissues to avoid xenorejection.

### Example 4 Generation of MUC15-, SIGLEC14-, TEX10-, and GPC4-Overexpressing Stem Cell Lines

This example describes the generation of human stem cell lines that have been genetically modified to overexpress one of the genes selected from MUC15, SIGLEC14, TEX10, GPC4.

H1 human embryonic stem cells from the “HM-KO” hESC line described in Example 1 (see Table 1) were transfected with the lentiviral vectors described in Example 2 encoding CD55, CD59, CD47, and Crry (mouse homolog for CD46). This cell line is referred to herein as either “HM-KO-Complement and Phagocytosis (noNK)”, “No NK”, or “BX1” (see TABLE 1).

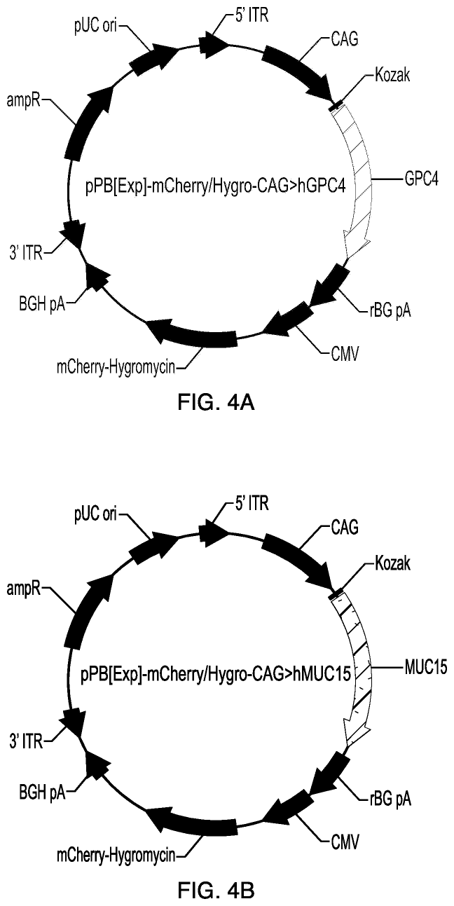

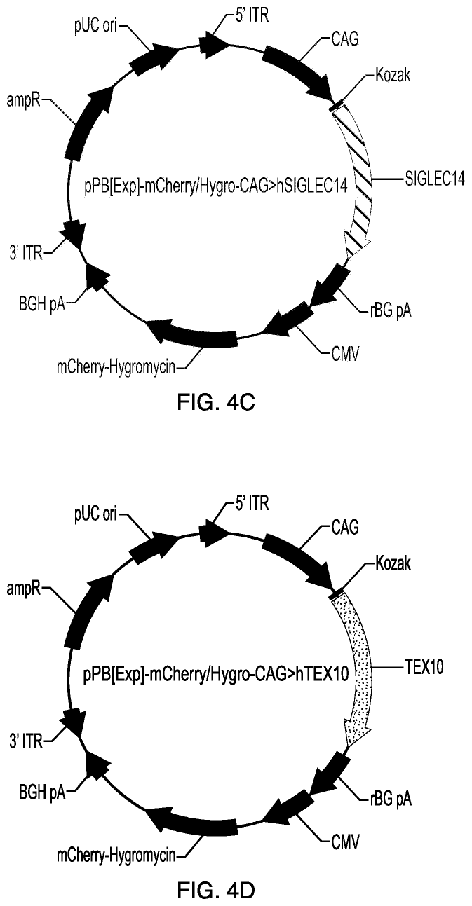

PiggyBac expression vectors were constructed which placed one of each of the genes (MUC15, SIGLEC14, TEX10, GPC4) under the control of the CAG promoter. Maps of the PiggyBac vectors are summarized in FIGS. 4A-4D. Briefly, each vector comprised the following elements interposed between flanking 5′ and 3′ PiggyBac inverted terminal repeat (ITR) sequences (listed in the 5′ to 3′ direction): a CAG promoter, a Kozak translation initiation sequence, one of the four indicated genes (as encoded by SEQ ID NOs: 1, 8, 11, and 15), a bovine growth hormone (BGH) polyadenylation signal, and mCherry and hygromycin marker genes under the control of the human cytomegalovirus immediate early promoter.

H1 human embryonic stem cells from the “HM-KO-Complement and Phagocytosis” (“No NK”) hESC line (see TABLE 1) were transfected with one of the four vectors described above. Briefly, 4×10⁵ accutase-dissociated “No NK” hESCs were plated onto a Cultrex (CTX; R&D Systems) pre-coated 12-well plate in Freedom Media (FRD1; ThermoFisher Custom) containing Thiazovivin (THZ; Sigma-Aldrich). A 200 μl transfection reaction was made by adding 1.6 μg hyPase (ET-11947), 2 μg of each respective plasmid, 2 μl of lipofectamine 2000 (STEMCELL Technologies) and OptiMEM I Reduced Serum Medium (Gibco, Cat #31985062). After incubating for 15 minutes at room temperature, the transfection reaction was added to the plated hESCs. After 48 hours, transfected cells were selected for via treatment with 20 μg/mL of hygromycin, which was increased stepwise to a final concentration of 90 μg/mL.

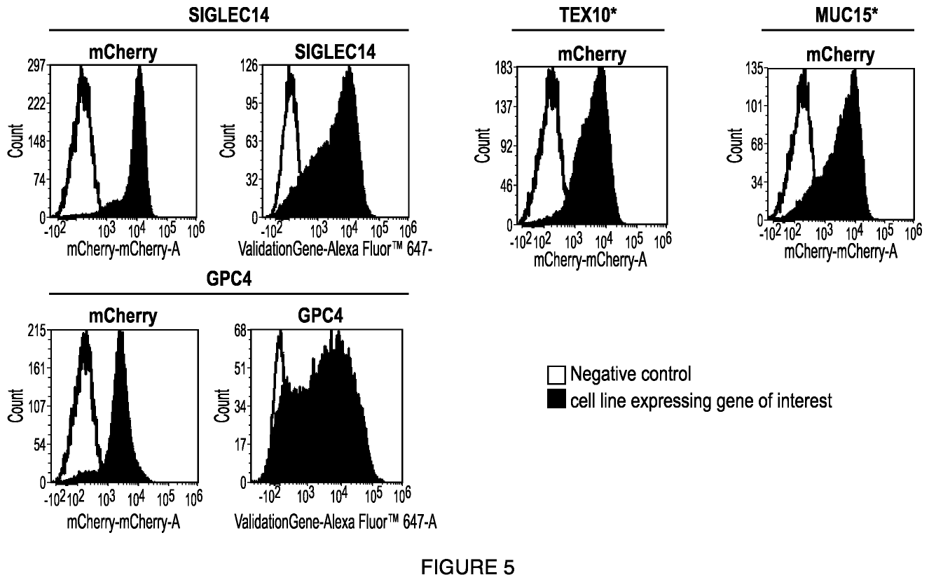

2-weeks post-selection, integration, and expression of the marker gene mCherry was validated in the four cell lines via flow cytometry. Additionally, membrane expression of SIGLEC14 and GPC4 was also validated in the respective modified cell lines via flow cytometry. Non-transfected cells from the parental “No NK” cell line were used as negative controls. Briefly, 500,000 cells were harvested and incubated in MACS buffer containing antibodies specific for SIGLEC5/SIGLEC14 (194128, BD) or GPC4 (FAB9195R, R&D Systems) and SytoxBlue live/dead stain. Analysis was performed on an Attune NxT. As shown in FIG. 5, mCherry expression was verified in all four cell lines, and membrane expression of SIGLEC14 and GPC4 was confirmed in the respective cell lines. The four cell lines are summarized in TABLE 2 below.

TABLE 2
Cell line information: BX1 derivatives
with an additional overexpressed gene

| | | Additional |
|---|---|---|
| Cell Line | Base Overexpressed Genes | Overexpressed Gene(s) |
| BX1.1 | CD46/Crry, CD55, CD59, CD47 | MUC15 |
| BX1.2 | CD46/Crry, CD55, CD59, CD47 | SIGLEC14 |
| BX1.3 | CD46/Crry, CD55, CD59, CD47 | TEX10 |
| BX1.4 | CD46/Crry, CD55, CD59, CD47 | GPC4 |

Expression of the four genes MUC15, SIGLEC14, TEX10, and GPC4 was analyzed via qRT-PCR in the four cell lines modified with the respective overexpression vectors. RNA was extracted from cells using RNeasy Micro Kit (Qiagen; Cat #74004) according to the manufacturer's protocol. 100 ng of RNA was used for reverse transcription using TaqPath-1 Multiplex Master Mix (Thermo Scientific; #A28525) according to the manufacturer's protocol. Amplification of GAPDH, TEX10, MUC15, GPC4 and SIGLEC14 was achieved by using ThermoFisher Scientific's TaqMan assays: GPC4 Hs00155059 TaqMan™ Gene Expression Assay (FAM), MUC15 Hs04971585_ml TaqMan™ Gene Expression Assay (FAM), SIGLEC14 Hs01592899_ml TaqMan™ Gene Expression Assay (FAM), and TEX10 Hs01076950_ml TaqMan™ Gene Expression Assay (FAM), respectively. Cycle threshold values were normalized with GAPDH and log fold change was calculated and normalized to that of the parental cell line (“No NK”).

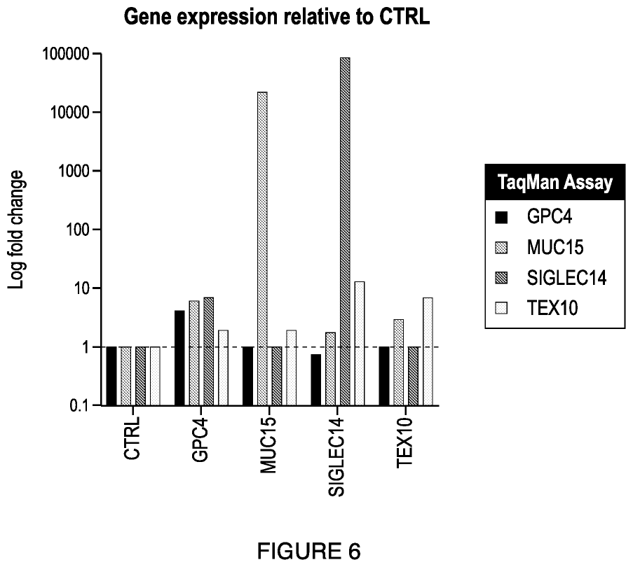

As summarized in FIG. 6, the cell lines modified to overexpress MUC15 and SIGLEC14 had higher expression of MUC15 and SIGLEC14, respectively. GPC4 and TEX10 expression was also increased in each line expressing these two genes, although overall expression was observed in the four lines, suggestive of endogenous expression of these two genes in the parental cell line.

### Example 5. Evaluation of Additional Immune Evasion Genes

This example evaluates the ability of stem cells modified to overexpress MUC15, SIGLEC14, TEX10, or GPC4 to evade immune rejection.

The four cell lines described in Example 4 were used to generate embryoid bodies (EBs), and the parental cell line BX1 was used as a control. 8,000 cells from each cell line were plated per well in a 96-well plate in STEMdiffTM APELTM2 Medium (STEMCELL Technologies) containing bFGF, BMP4, VEGF (R&D Systems), SCF (PeproTech), and Y-27632 (Reprocell). The cultures were left untouched for six days, at which point EBs were collected.

The embryoid bodies derived from the genetically modified cell lines were transplanted into mice. Briefly, C57B16/J mice were anesthetized with 1-3% isoflurane by inhalation and maintained during the surgical procedure. Eye lube was applied to prevent drying. The surgical site was shaved and disinfected with betadine and alcohol swabs. 80 EBs were loaded into a syringe with a silicone catheter. A 5 mm skin and peritoneum incision was performed on the lower left flank of mice. The left kidney was isolated, and EBs were transplanted under the capsule. Carprofen was injected intraperitoneal during surgery to reduce post-operation discomfort. The peritoneal layer was closed with a 4-0 absorbable suture. The skin incision was closed with three surgical staples. Mice were injected with a second dose of carprofen 1 day after the procedure to alleviate any post-operation discomfort. Mice were inspected immediately following the procedure and 1-day post-procedure to ensure they had made a full recovery.

8 weeks following the procedure, mice were euthanized with CO₂ gas. Necropsy was performed on a dissection tray using surgical instruments. To obtain samples for immunohistochemistry (IHC) analysis, mice were bled through the inferior vena cava and perfused with PBS and 4% PFA through the left ventricle of the heart. Kidneys were excised and fixed in 4% PFA for 48-72 hours. Both left and right kidneys were harvested. The left kidney was used as the experimental sample, and the right kidney was used as the control sample.

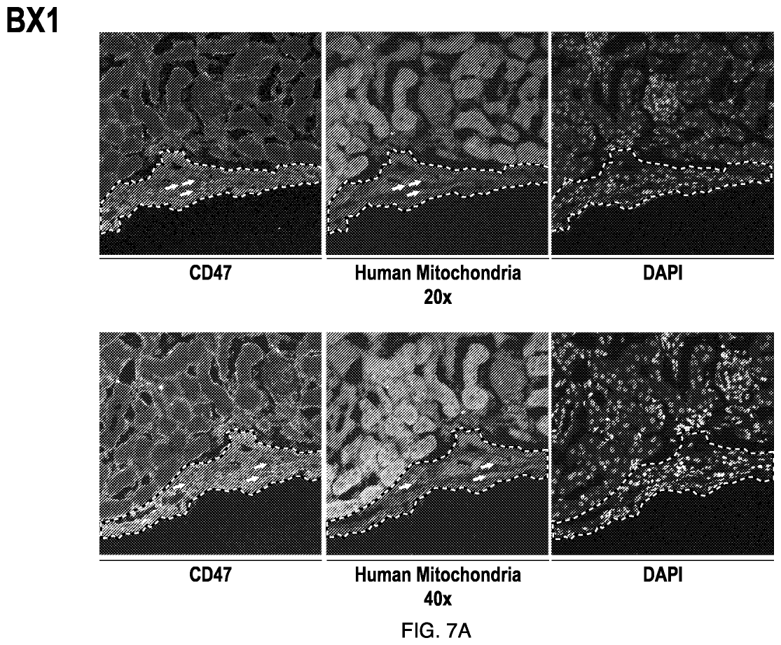

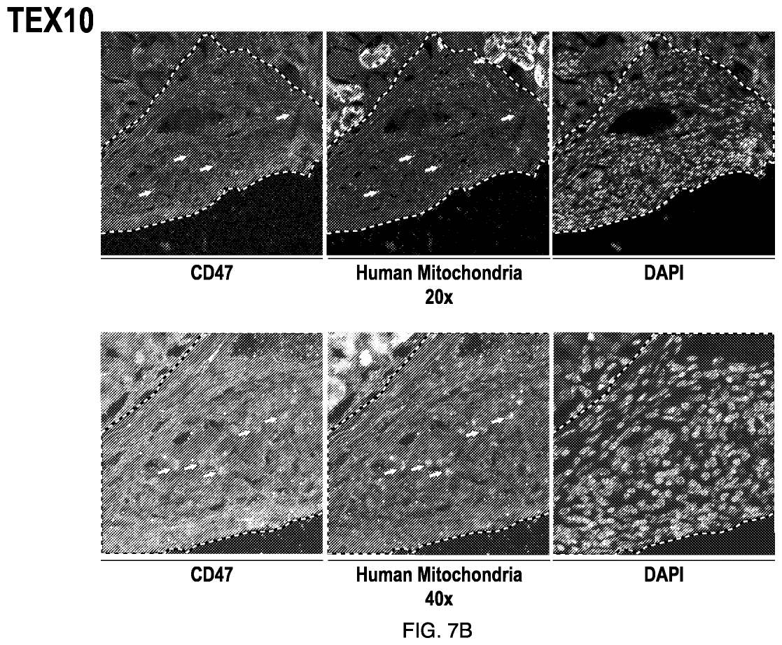

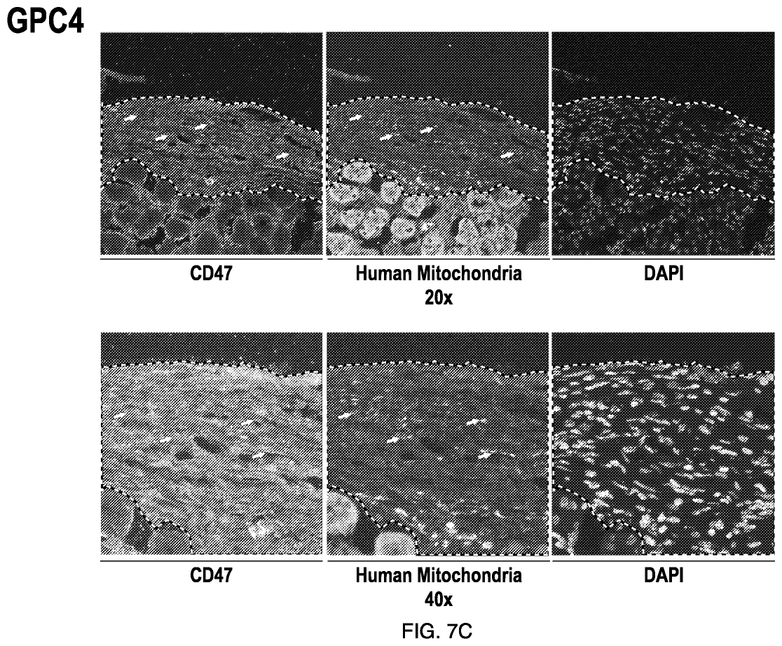

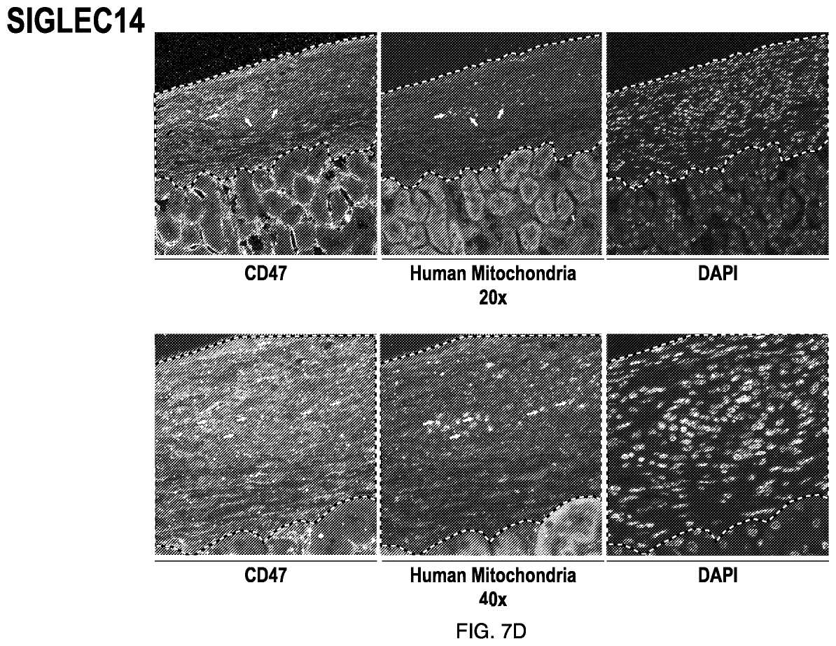

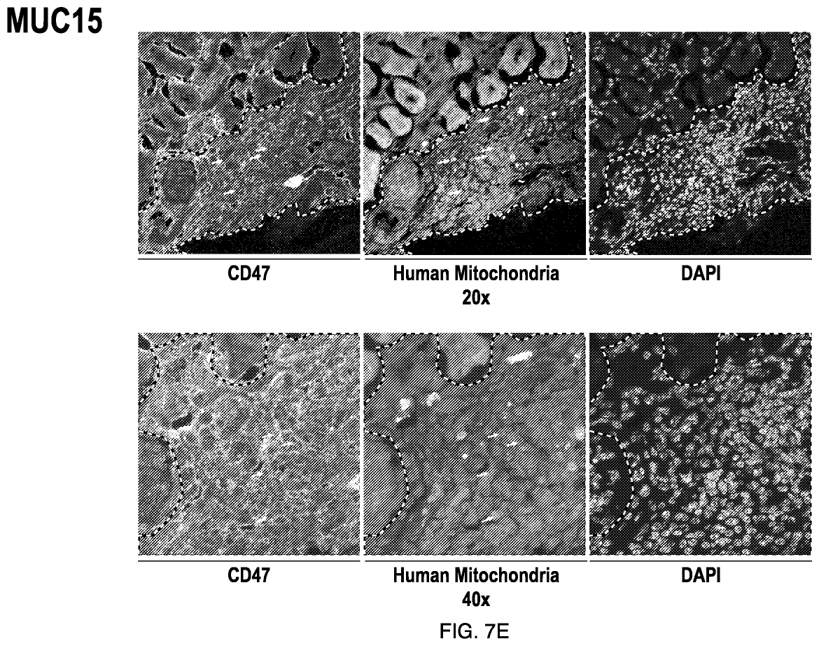

Kidneys transplanted with EBs derived from the genetically modified cells were harvested, cryoprotected via sucrose gradient, and embedded in Tissue-Tek Optimal Cutting Temperature Compound (OCT) (Sakura Finetek). Kidneys were serially sectioned at 5 microns with a Leica CM3050S and adhered onto Superfrost Plus slides (Fisherbrand). Blocking and permeabilization buffer containing 5% normal donkey serum (Jackson Immunoresearch), 0.1% Triton X-100 (Sigma-Aldrich), and 100 mM glycine (Sigma-Aldrich) was prepared. Sections were incubated for 30 minutes at room temperature and primary antibodies were prepared in blocking buffer to previously established dilutions. Kidney sections were incubated overnight at 4° C. with primary antibody specific for mouse CD47 (R&D) and human mitochondria (113-1, R&D). Slides were washed with PBS and secondary antibodies prepared in blocking buffer (details are included in table below). Slides were incubated for 1 hour at room temperature in the dark. Slides were wash with PBS and mounted with ProLong Gold with DAPI (Invitrogen). Coverslips (Epredia) were allowed to cure overnight and imaged via confocal microscopy on a Zeiss LSM 800. The resulting images were analyzed with ImageJ. Exemplary IHC images of kidney sections from mice transplanted with EBs derived from the TEX10-overexpressing cells, the GPC4-overexpressing cells, the SIGLEC14-overexpressing cells, and the MUC15-overexpressing cells are shown in FIGS. 7B, 7C, 7D, and 7E, respectively. IHC images of kidney sections from mice injected with EBs derived from the parental cell line are shown in FIG. 7A.

Cell lines that were modified to overexpress TEX10, GPC4, SIGLEC14, or MUC15 had larger grafts when compared to the parental BX-1 line (FIG. 4). The cell line overexpressing TEX10 had a strong mitochondrial and CD47 signal, and the graft spread laterally under the kidney capsule. The morphology of the GPC4-overexpressing cells in the graft showed a more elongated shape when compared to the other grafts, and both CD47 and mitochondrial staining were present. The graft of the SIGLEC14-overexpressing cell line spread out more underneath the capsule, and there was strong CD47 and mitochondrial signal present in the graft. The MUC15-overexpressing cell line had deeper infiltration than the previous grafts. There was bright CD47 signal, and the mitochondrial signal was present.

These results suggest that stem cells modified to overexpress MUC15, SIGLEC14, TEX10, or GPC4 can evade immune rejection.

### Example 6. Evaluation of Gene Combinations for Stem Cell Modification

Stem cells modified to express certain genes or gene combinations are evaluated for their potential in evading immune rejections.

The HM-KO hESC line is modified to overexpress the gene or gene combination, as shown in TABLE 1 above. The desired gene or gene combination is transduced into HM-KO cells via one or more lentiviral vectors. E.g., if more than one gene is introduced into HM-KO cells, these genes may be constructed in a single lentiviral vector or divided up (in any way) and constructed in multiple lentiviral vectors. Further, if more than one lentiviral vector is used to deliver the desired genes, HM-KO cells may be transduced with the vectors all at once or in multiple cycles.

Complement deposition, cytotoxicity, and phagocytosis assays are performed to evaluate whether stem cells modified to express the gene or gene combination can evade the immune system in vitro. Attenuated complement deposition, cytotoxicity, and/or phagocytosis compared to wild-type (WT) or HM-KO hESCs would suggest that expression of that gene or gene combination improves the ability of cells to evade the immune system.

To evaluate whether stems cells modified to express the gene or gene combination have the ability evade the immune system in vivo, such cells are transplanted into immunocompetent mice. The survival of these transplanted cells, e.g., the ability to develop as teratomas, is monitored. Additionally, the modified stem cells are differentiated into tissues, which are then transplanted into immunocompetent mice. The survival and function of the tissues are monitored.

Development of teratomas in mice transplanted with cell lines overexpressing a gene or gene combination would suggest that expression of that gene or gene combination improves the ability of cells to evade the immune system, the same for if the transplanted tissues are able to survive and/or functional.

### Example 7. Evaluation of Additional Immune Evasion Genes

The genes MUC15, SIGLEC14, TEX10, and GPC4, are further evaluated for their ability to improve the ability of cells to evade the immune system.

The HM-KO-Complement and Phagocytosis (noNK) line (i.e., BX1) is genetically modified to overexpress combinations of MUC15, SIGLEC14, TEX10, and/or GPC4 to yield modified stem cells, as shown in TABLE 3. The modification may be achieved, e.g., with the method described in Example 2 or Example 4.

TABLE 3
Cell line information

| | | Additional |
|---|---|---|
| Cell Line | Base Overexpressed Genes | Overexpressed Gene(s) |
| BX1.12 | CD46/Crry, CD55, CD59, CD47 | MUC15, SIGLEC14 |
| BX1.13 | CD46/Crry, CD55, CD59, CD47 | MUC15, TEX10 |
| BX1.14 | CD46/Crry, CD55, CD59, CD47 | MUC15, GPC4 |
| BX1.23 | CD46/Crry, CD55, CD59, CD47 | SIGLEC14, TEX10 |
| BX1.24 | CD46/Crry, CD55, CD59, CD47 | SIGLEC14, GPC4 |
| BX1.34 | CD46/Crry, CD55, CD59, CD47 | TEX10, GPC4 |
| BX1.123 | CD46/Crry, CD55, CD59, CD47 | MUC15, SIGLEC14, TEX10 |
| BX1.124 | CD46/Crry, CD55, CD59, CD47 | MUC15, SIGLEC14, GPC4 |
| BX1.134 | CD46/Crry, CD55, CD59, CD47 | MUC15, TEX10, GPC4 |
| BX1.234 | CD46/Crry, CD55, CD59, CD47 | SIGLEC14, TEX10, GPC4 |
| BX1.1234 | CD46/Crry, CD55, CD59, CD47 | MUC15, SIGLEC14, TEX10, GPC4 |

Complement deposition, cytotoxicity, and phagocytosis assays are performed on stem cells from cell lines BX1.1 through BX1.4 (TABLE 2) and/or on the cell lines listed in TABLE 3 to evaluate whether stem cells modified to express the gene or gene combination have the ability to evade the immune system in vitro. Attenuated complement deposition, cytotoxicity, and/or phagocytosis compared to wild-type (WT) or HM-KO hESCs would suggest that expression of that gene or gene combination improves the ability of cells to evade the immune system.

To evaluate whether expression of the additional genes (MUC15, SIGLEC14, TEX10, GPC4, or a combination thereof) improves the ability of cells to evade the immune system in vivo, cells from cell lines BX1.1 through BX1.4 (TABLE 2) or from the cell lines listed in TABLE 3 are transplanted into immunocompetent mice. The survival of these cells after transplantation, e.g., the ability to develop as teratomas, is monitored. The modified stem cells are also differentiated into tissues, which are then transplanted into immunocompetent mice. The survival and function of the tissues are monitored.

Development of teratomas in mice transplanted with cell lines overexpressing a gene or combination of genes would suggest that expression of that gene or combination of genes improves the ability of cells to evade the immune system, the same for if the transplanted tissues are able to survive and/or functional.

### Example 8. Treatment of Type 1 Diabetes with Modified Cells

Stem cells from cell lines BX1.1 through BX1.4 (TABLE 2) or from cell lines listed in TABLE 3 are differentiated into pancreatic beta cells or beta-like cells. The differentiated cells are implanted into a mammalian subject (e.g., a murine subject useful as a model of type 1 diabetes or a human subject having or at risk of having type 1 diabetes). Where appropriate, differentiated cells derived from stem cells lacking one or more of the genetic modifications of any one of cells lines in TABLE 2 or TABLE 3 are administered to a subject as a control. Insulin and glucose levels in the subject and/or symptoms of diabetes in the subject are measured in the weeks and/or months following transplantation of the differentiated cells. It is contemplated that glucose levels in the subject and/or one or more symptoms of diabetes in the subject will be improved following transplantation of the experimental differentiated cells, e.g., as compared to pre-transplantation glucose levels or symptoms in the subject, and/or as compared to those of an appropriate control subject that is either administered a control treatment or that is not administered the experimental differentiated cells.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

SEQUENCE LISTING

| SEQ | |
|---|---|
| ID NO | Sequence |
| 1 | ATGGGCATAATACAATCTATTCTTGCCACATCAAG |
| | GGATTGTTATTCCTTTAAAAAAAAACCAATACCAA |
| | AGAAGCCTACAATGTTGGCCTTAGCCAAAATTCTG |
| | TTGATTTCAACGTTGTTTTATTCACTTCTATCGGG |
| | GAGCCATGGAAAAGAAAATCAAGACATAAACACAA |
| | CACAGAACATTGCAGAAGTTTTTAAAACAATGGAA |
| | AATAAACCTATTTCTTTGGAAAGTGAAGCAAACTT |
| | AAACTCAGATAAAGAAAATATAACCACCTCAAATC |
| | TCAAGGCGAGTCATTCCCCTCCTTTGAATCTACCC |
| | AACAACAGCCACGGAATAACAGATTTCTCCAGTAA |
| | CTCATCAGCAGAGCATTCTTTGGGCAGTCTAAAAC |
| | CCACATCTACCATTTCCACAAGCCCTCCCTTGATC |
| | CATAGCTTTGTTTCTAAAGTGCCTTGGAATGCACC |
| | TATAGCAGATGAAGATCTTTTGCCCATCTCAGCAC |
| | ATCCCAATGCTACACCTGCTCTGTCTTCAGAAAAC |
| | TTCACTTGGTCTTTGGTCAATGACACCGTGAAAAC |
| | TCCTGATAACAGTTCCATTACAGTTAGCATCCTCT |
| | CTTCAGAACCAACTTCTCCATCTGTGACCCCCTTG |
| | ATAGTGGAACCAAGTGGATGGCTTACCACAAACAG |
| | TGATAGCTTCACTGGGTTTACCCCTTATCAAGAAA |
| | AAACAACTCTACAGCCTACCTTAAAATTCACCAAT |
| | AATTCAAAACTCTTTCCAAATACGTCAGATCCCCA |
| | AAAAGAAAATAGAAATACAGGAATAGTATTCGGGG |
| | CCATTTTAGGTGCTATTCTGGGTGTCTCATTGCTT |
| | ACTCTTGTGGGCTACTTGTTGTGTGGAAAAAGGAA |
| | AACGGATTCATTTTCCCATCGGCGACTTTATGACG |
| | ACAGAAATGAACCAGTTCTGCGATTAGACAATGCA |
| | CCGGAACCTTATGATGTGAGTTTTGGGAATTCTAG |
| | CTACTACAATCCAACTTTGAATGATTCAGCCATGC |
| | CAGAAAGTGAAGAAAATGCACGTGATGGCATTCCT |
| | ATGGATGACATACCTCCACTTCGTACTTCTGTATA |
| | G |
| 2 | MGIIQSILATSRDCYSFKKKPIPKKPTMLALAKIL |
| | LISTLFYSLLSGSHGKENQDINTTQNIAEVFKTME |
| | NKPISLESEANLNSDKENITTSNLKASHSPPLNLP |
| | NNSHGITDESSNSSAEHSLGSLKPTSTISTSPPLI |
| | HSFVSKVPWNAPIADEDLLPISAHPNATPALSSEN |
| | FTWSLVNDTVKTPDNSSITVSILSSEPTSPSVTPL |
| | IVEPSGWLTTNSDSFTGFTPYQEKTTLQPTLKFIN |
| | NSKLFPNTSDPQKENRNTGIVFGAILGAILGVSLL |
| | TLVGYLLCGKRKTDSFSHRRLYDDRNEPVLRLDNA |
| | PEPYDVSFGNSSYYNPTLNDSAMPESEENARDGIP |
| | MDDIPPLRTSV |
| 3 | ATGGGCATAATACAATCTATTCTTGCCACATCAAG |
| | GGATTGTTATTCCTTTAAAAAAAAACCAATACCAA |
| | AGAAGCCTACAATGTTGGCCTTAGCCAAAATTCTG |
| | TTGATTTCAACGTTGTTTTATTCACTTCTATCGGG |
| | GAGCCATGGAAAAGAAAATCAAGACATAAACACAA |
| | CACAGAACATTGCAGAAGTTTTTAAAACAATGGAA |
| | AATAAACCTATTTCTTTGGAAAGTGAAGCAAACTT |
| | AAACTCAGATAAAGAAAATATAACCACCTCAAATC |
| | TCAAGGCGAGTCATTCCCCTCCTTTGAATCTACCC |
| | AACAACAGCCACGGAATAACAGATTTCTCCAGTAA |
| | CTCATCAGCAGAGCATTCTTTGGGCAGTCTAAAAC |
| | CCACATCTACCATTTCCACAAGCCCTCCCTTGATC |
| | CATAGCTTTGTTTCTAAAGTGCCTTGGAATGCACC |
| | TATAGCAGATGAAGATCTTTTGCCCATCTCAGCAC |
| | ATCCCAATGCTACACCTGCTCTGTCTTCAGAAAAC |
| | TTCACTTGGTCTTTGGTCAATGACACCGTGAAAAC |
| | TCCTGATAACAGTTCCATTACAGTTAGCATCCTCT |
| | CTTCAGAACCAACTTCTCCATCTGTGACCCCCTTG |
| | ATAGTGGAACCAAGTGGATGGCTTACCACAAACAG |
| | TGATAGCTTCACTGGGTTTACCCCTTATCAAGAAA |
| | AAACAACTCTACAGCCTACCTTAAAATTCACCAAT |
| | AATTCAAAACTCTTTCCAAATACGTCAGATCCCCA |
| | AAAAGTTCTGCGATTAGACAATGCACCGGAACCTT |
| | ATGATGTGAGTTTTGGGAATTCTAGCTACTACAAT |
| | CCAACTTTGAATGATTCAGCCATGCCAGAAAGTGA |
| | AGAAAATGCACGTGATGGCATTCCTATGGATGACA |
| | TACCTCCACTTCGTACTTCTGTATAG |
| 4 | MGIIQSILATSRDCYSFKKKPIPKKPTMLALAKIL |
| | LISTLFYSLLSGSHGKENQDINTTQNIAEVEKTME |
| | NKPISLESEANLNSDKENITTSNLKASHSPPLNLP |
| | NNSHGITDESSNSSAEHSLGSLKPTSTISTSPPLI |
| | HSFVSKVPWNAPIADEDLLPISAHPNATPALSSEN |
| | FTWSLVNDTVKTPDNSSITVSILSSEPTSPSVTPL |
| | IVEPSGWLTTNSDSFTGFTPYQEKTTLQPTLKFIN |
| | NSKLFPNTSDPQKVLRLDNAPEPYDVSFGNSSYYN |
| | PTLNDSAMPESEENARDGIPMDDIPPLRTSV |
| 5 | ATGTTGGCCTTAGCCAAAATTCTGTTGATTTCAAC |
| | GTTGTTTTATTCACTTCTATCGGGGAGCCATGGAA |
| | AAGAAAATCAAGACATAAACACAACACAGAACATT |
| | GCAGAAGTTTTTAAAACAATGGAAAATAAACCTAT |
| | TTCTTTGGAAAGTGAAGCAAACTTAAACTCAGATA |
| | AAGAAAATATAACCACCTCAAATCTCAAGGCGAGT |
| | CATTCCCCTCCTTTGAATCTACCCAACAACAGCCA |
| | CGGAATAACAGATTTCTCCAGTAACTCATCAGCAG |
| | AGCATTCTTTGGGCAGTCTAAAACCCACATCTACC |
| | ATTTCCACAAGCCCTCCCTTGATCCATAGCTTTGT |
| | TTCTAAAGTGCCTTGGAATGCACCTATAGCAGATG |
| | AAGATCTTTTGCCCATCTCAGCACATCCCAATGCT |
| | ACACCTGCTCTGTCTTCAGAAAACTTCACTTGGTC |
| | TTTGGTCAATGACACCGTGAAAACTCCTGATAACA |
| | GTTCCATTACAGTTAGCATCCTCTCTTCAGAACCA |
| | ACTTCTCCATCTGTGACCCCCTTGATAGTGGAACC |
| | AAGTGGATGGCTTACCACAAACAGTGATAGCTTCA |
| | CTGGGTTTACCCCTTATCAAGAAAAAACAACTCTA |
| | CAGCCTACCTTAAAATTCACCAATAATTCAAAACT |
| | CTTTCCAAATACGTCAGATCCCCAAAAAGAAAATA |
| | GAAATACAGGAATAGTATTCGGGGCCATTTTAGGT |
| | GCTATTCTGGGTGTCTCATTGCTTACTCTTGTGGG |
| | CTACTTGTTGTGTGGAAAAAGGAAAACGGATTCAT |
| | TTTCCCATCGGCGACTTTATGACGACAGAAATGAA |
| | CCAGTTCTGCGATTAGACAATGCACCGGAACCTTA |
| | TGATGTGAGTTTTGGGAATTCTAGCTACTACAATC |
| | CAACTTTGAATGATTCAGCCATGCCAGAAAGTGAA |
| | GAAAATGCACGTGATGGCATTCCTATGGATGACAT |
| | ACCTCCACTTCGTACTTCTGTATAG |
| 6 | MLALAKILLISTLFYSLLSGSHGKENQDINTTQNI |
| | AEVFKTMENKPISLESEANLNSDKENITTSNLKAS |
| | HSPPLNLPNNSHGITDFSSNSSAEHSLGSLKPTST |
| | ISTSPPLIHSFVSKVPWNAPIADEDLLPISAHPNA |
| | TPALSSENFTWSLVNDTVKTPDNSSITVSILSSEP |
| | TSPSVTPLIVEPSGWLTTNSDSFTGFTPYQEKTTL |
| | QPTLKFINNSKLFPNTSDPQKENRNTGIVEGAILG |
| | AILGVSLLTLVGYLLCGKRKTDSFSHRRLYDDRNE |
| | PVLRLDNAPEPYDVSFGNSSYYNPTLNDSAMPESE |
| | ENARDGIPMDDIPPLRTSV |
| 7 | KENQDINTTQNIAEVEKTMENKPISLESEANLNSD |
| | KENITTSNLKASHSPPLNLPNNSHGITDESSNSSA |
| | EHSLGSLKPTSTISTSPPLIHSFVSKVPWNAPIAD |
| | EDLLPISAHPNATPALSSENFTWSLVNDTVKTPDN |
| | SSITVSILSSEPTSPSVTPLIVEPSGWLTTNSDSF |
| | TGFTPYQEKTTLQPTLKFINNSKLFPNTSDPQKEN |
| | RNTGIVEGAILGAILGVSLLTLVGYLLCGKRKTDS |
| | FSHRRLYDDRNEPVLRLDNAPEPYDVSFGNSSYYN |
| | PTLNDSAMPESEENARDGIPMDDIPPLRTSV |
| 8 | ATGCTGCCCCTGCTGCTGCTGCCCCTGCTGTGGGG |
| | GGGGTCCCTGCAGGAGAAGCCAGTGTACGAGCTGC |
| | AAGTGCAGAAGTCGGTGACGGTGCAGGAGGGCCTG |
| | TGCGTCCTTGTGCCCTGCTCCTTCTCTTACCCCTG |
| | GAGATCCTGGTATTCCTCTCCCCCACTCTACGTCT |
| | ACTGGTTCCGGGACGGGGAGATCCCATACTACGCT |
| | GAGGTTGTGGCCACAAACAACCCAGACAGAAGAGT |
| | GAAGCCAGAGACCCAGGGCCGATTCCGCCTCCTTG |
| | GGGATGTCCAGAAGAAGAACTGCTCCCTGAGCATC |
| | GGAGATGCCAGAATGGAGGACACGGGAAGCTATTT |
| | CTTCCGCGTGGAGAGAGGAAGGGATGTAAAATATA |
| | GCTACCAACAGAATAAGCTGAACTTGGAGGTGACA |
| | GCCCTGATAGAGAAACCCGACATCCACTTTCTGGA |
| | GCCTCTGGAGTCCGGCCGCCCCACAAGGCTGAGCT |
| | GCAGCCTTCCAGGATCCTGTGAAGCGGGACCACCT |
| | CTCACATTCTCCTGGACGGGGAATGCCCTCAGCCC |
| | CCTGGACCCCGAGACCACCCGCTCCTCGGAGCTCA |
| | CCCTCACCCCCAGGCCCGAGGACCATGGCACCAAC |
| | CTCACCTGTCAGGTGAAACGCCAAGGAGCTCAGGT |
| | GACCACGGAGAGAACTGTCCAGCTCAATGTCTCCT |
| | ATGCTCCACAGAACCTCGCCATCAGCATCTTCTTC |
| | AGAAATGGCACAGGCACAGCCCTGCGGATCCTGAG |
| | CAATGGCATGTCGGTGCCCATCCAGGAGGGCCAGT |
| | CCCTGTTCCTCGCCTGCACAGTTGACAGCAACCCC |
| | CCTGCCTCACTGAGCTGGTTCCGGGAGGGAAAAGC |
| | CCTCAATCCTTCCCAGACCTCAATGTCTGGGACCC |
| | TGGAGCTGCCTAACATAGGAGCTAGAGAGGGAGGG |
| | GAATTCACCTGCCGGGTTCAGCATCCGCTGGGCTC |
| | CCAGCACCTGTCCTTCATCCTTTCTGTGCAGAGAA |
| | GCTCCTCTTCCTGCATATGTGTAACTGAGAAACAG |
| | CAGGGCTCCTGGCCCCTCGTCCTCACCCTGATCAG |
| | GGGGGCTCTCATGGGGGCTGGCTTCCTCCTCACCT |
| | ATGGCCTCACCTGGATCTACTATACCAGGTGTGGA |
| | GGCCCCCAGCAGAGCAGGGCTGAGAGGCCTGGCTG |
| | A |
| 9 | MLPLLLLPLLWGGSLQEKPVYELQVQKSVTVQEGL |
| | CVLVPCSFSYPWRSWYSSPPLYVYWERDGEIPYYA |
| | EVVATNNPDRRVKPETQGRFRLLGDVQKKNCSLSI |
| | GDARMEDTGSYFFRVERGRDVKYSYQQNKLNLEVT |
| | ALIEKPDIHFLEPLESGRPTRLSCSLPGSCEAGPP |
| | LTFSWTGNALSPLDPETTRSSELTLTPRPEDHGTN |
| | LTCQVKRQGAQVTTERTVQLNVSYAPQNLAISIFF |
| | RNGTGTALRILSNGMSVPIQEGQSLFLACTVDSNP |
| | PASLSWFREGKALNPSQTSMSGTLELPNIGAREGG |
| | EFTCRVQHPLGSQHLSFILSVQRSSSSCICVTEKQ |
| | QGSWPLVLTLIRGALMGAGFLLTYGLTWIYYTRCG |
| | GPQQSRAERPG |
| 10 | EKPVYELQVQKSVTVQEGLCVLVPCSFSYPWRSWY |
| | SSPPLYVYWERDGEIPYYAEVVATNNPDRRVKPET |
| | QGRFRLLGDVQKKNCSLSIGDARMEDTGSYFFRVE |
| | RGRDVKYSYQQNKLNLEVTALIEKPDIHFLEPLES |
| | GRPTRLSCSLPGSCEAGPPLTFSWTGNALSPLDPE |
| | TTRSSELTLTPRPEDHGTNLTCQVKRQGAQVTTER |
| | TVQLNVSYAPQNLAISIFFRNGTGTALRILSNGMS |
| | VPIQEGQSLFLACTVDSNPPASLSWFREGKALNPS |
| | QTSMSGTLELPNIGAREGGEFTCRVQHPLGSQHLS |
| | FILSVQRSSSSCICVTEKQQGSWPLVLTLIRGALM |
| | GAGFLLTYGLTWIYYTRCGGPQQSRAERPG |
| 11 | ATGACTAAAAAAAGAAAACGCCAACATGATTTTCA |
| | AAAAGTAAAATTGAAAGTTGGTAAAAAGAAGCCCA |
| | AGTTACAAAATGCTACTCCTACAAACTTTAAAACA |
| | AAGACTATACATCTGCCTGAGCAACTCAAAGAGGA |
| | TGGAACACTTCCAACAAACAATAGAAAACTTAACA |
| | TAAAGGATTTGCTGTCACAGATGCATCACTACAAT |
| | GCTGGGGTTAAACAAAGTGCTCTTCTTGGACTTAA |
| | AGACCTTTTGTCTCAATACCCATTTATAATTGATG |
| | CACACCTTTCAAACATATTAAGTGAAGTGACTGCT |
| | GTGTTTACAGATAAAGATGCTAATGTACGATTAGC |
| | AGCAGTTCAACTTCTTCAATTCCTGGCCCCCAAAA |
| | TACGAGCTGAACAAATTTCTCCATTTTTTCCTTTG |
| | GTAAGTGCCCATCTCTCTAGTGCCATGACTCACAT |
| | TACTGAAGGAATTCAGGAGGACTCTTTAAAAGTTT |
| | TGGACATTCTGCTGGAACAGTACCCAGCTCTAATT |
| | ACTGGCCGTAGCAGCATATTGCTTAAGAATTTTGT |
| | AGAACTTATTTCTCATCAGCAGCTGTCCAAAGGAC |
| | TGATAAATAGAGACAGATCCCAGTCCTGGATACTT |
| | TCTGTAAATCCTAATCGGAGACTCACTTCTCAGCA |
| | ATGGAGGCTGAAAGTCTTAGTGAGACTCAGTAAAT |
| | TCCTTCAGGCCTTGGCAGATGGATCCAGTAGGTTG |
| | AGAGAAAGTGAAGGACTTCAGGAACAGAAAGAAAA |
| | TCCCCATGCCACTAGCAACTCCATTTTTATCAACT |
| | GGAAGGAACATGCCAACGACCAGCAACACATCCAG |
| | GTTTATGAAAATGGGGGTTCACAGCCAAATGTCAG |
| | TTCACAGTTCAGGCTACGGTATCTGGTTGGAGGAC |
| | TGAGTGGTGTGGATGAAGGCCTGTCATCTACTGAA |
| | AACCTGAAAGGATTTATTGAGATAATAATTCCATT |
| | GCTAATTGAATGCTGGGTTGAAGCTGTACCTCCAC |
| | AACTAGCTACTCCTGTTGGGAATGGTATAGAACGA |
| | GAACCTCTACAGGTTATGCAGCAAGTTCTTAATAT |
| | TATTTCCCTTCTGTGGAAACTCTCTAAACAACAGG |
| | ATGAAACCCATAAATTGGAGTCATGGCTTCGAAAG |
| | AACTACCTTATTGATTTTAAACACCATTTTATGAG |
| | TCGTTTTCCATATGTCTTAAAAGAAATAACCAAGC |
| | ACAAAAGGAAAGAGCCAAATAAAAGCATCAAGCAT |
| | TGCACAGTTCTCTCCAATAACATAGATCATCTCTT |
| | ACTGAATTTAACACTGTCTGATATCATGGTCTCCC |
| | TGGCAAATGCGTCAACTTTGCAGAAGGATTGCAGT |
| | TGGATAGAAATGATAAGGAAATTTGTAACAGAGAC |
| | CCTTGAAGATGGCTCTAGGCTAAATAGTAAGCAAC |
| | TGAACAGATTGCTGGGAGTATCCTGGAGGTTAATG |
| | CAAATACAGCCAAACAGAGAGGACACAGAGACTCT |
| | TATTAAGGCAGTTTATACATTATATCAGCAGAGGG |
| | GCCTTATCCTTCCAGTTCGGACTTTGTTATTGAAG |
| | TTTTTCAGTAAAATCTATCAGACAGAAGAACTGAG |
| | ATCTTGTAGATTCAGATATCGTAGTAAAGTGTTAT |
| | CCCGTTGGCTGGCTGGCTTACCATTGCAACTTGCT |
| | CATCTTGGCTCCCGAAATCCTGAGCTCTCTACACA |
| | GCTTATCGATATCATTCATACCGCTGCAGCACGAG |
| | CAAATAAAGAATTACTAAAAAGTTTACAAGCTACT |
| | GCCCTCCGAATTTATGATCCACAAGAAGGTGCTGT |
| | GGTGGTTCTCCCTGCAGACTCTCAGCAGCGTTTGG |
| | TTCAGCTTGTATATTTCCTACCCAGTCTGCCGGCT |
| | GATTTGCTTTCTCGGTTAAGTCGTTGCTGTATTAT |
| | GGGAAGACTCAGTTCAAGTTTGGCTGCCATGCTTA |
| | TCGGGATACTGCACATGAGATCATCATTTTCTGGG |
| | TGGAAGTATTCAGCTAAAGACTGGTTGATGAGTGA |
| | TGTAGACTATTTCAGCTTCTTATTTTCCACACTTA |
| | CAGGGTTTTCGAAAGAGGAGTTGACTTGGCTTCAG |
| | AGCCTTCGAGGAGTTCCTCATGTCATCCAGACACA |
| | GCTTTCCCCTGTGCTTCTCTACCTTACAGATTTGG |
| | ATCAATTTTTACACCACTGGGATGTAACAGAGGCA |
| | GTTTTTCACAGTTTATTGGTTATTCCTGCCCGAAG |
| | TCAGAACTTTGACATCTTGCAAAGTGCCATCAGTA |
| | AGCATTTGGTTGGGTTGACTGTAATTCCTGACAGC |
| | ACGGCTGGCTGTGTTTTTGGTGTTATCTGTAAGCT |
| | CCTGGATCATACTTGTGTAGTTAGTGAGACTCTAC |
| | TGCCATTTCTGGCTTCTTGTTGCTACAGTCTTCTT |
| | TATTTTCTGCTCACTATAGAGAAAGGGGAAGCAGA |
| | ACATCTAAGAAAGAGGGACAAGCTGTGGGGGGTCT |
| | GTGTCTCCATCCTGGCTCTCTTGCCTCGAGTCCTC |
| | AGGTTGATGCTGCAGAGCCTGCGGGTGAACAGAGT |
| | TGGGCCTGAGGAGCTGCCTGTTGTGGGCCAGCTGC |
| | TTCGACTGCTGCTTCAGCATGCACCCCTCAGGACT |
| | CATATGTTGACCAATGCGATCTTGGTGCAGCAGAT |
| | CATCAAGAATATCACGACATTGAAGAGTGGAAGTG |
| | TTCAGGAACAGTGGCTCACAGACTTACATTACTGC |
| | TTCAACGTGTATATCACTGGGCATCCCCAAGGGCC |
| | CAGTGCACTGGCTACAGTGTATTGA |
| 12 | MTKKRKRQHDFQKVKLKVGKKKPKLQNATPTNFKT |
| | KTIHLPEQLKEDGTLPTNNRKLNIKDLLSQMHHYN |
| | AGVKQSALLGLKDLLSQYPFIIDAHLSNILSEVTA |
| | VFTDKDANVRLAAVQLLQFLAPKIRAEQISPFFPL |
| | VSAHLSSAMTHITEGIQEDSLKVLDILLEQYPALI |
| | TGRSSILLKNFVELISHQQLSKGLINRDRSQSWIL |
| | SVNPNRRLTSQQWRLKVLVRLSKFLQALADGSSRL |
| | RESEGLQEQKENPHATSNSIFINWKEHANDQQHIQ |
| | VYENGGSQPNVSSQFRLRYLVGGLSGVDEGLSSTE |
| | NLKGFIEIIIPLLIECWVEAVPPQLATPVGNGIER |
| | EPLQVMQQVLNIISLLWKLSKQQDETHKLESWLRK |
| | NYLIDFKHHEMSREPYVLKEITKHKRKEPNKSIKH |
| | CTVLSNNIDHLLLNLTLSDIMVSLANASTLQKDCS |
| | WIEMIRKFVTETLEDGSRLNSKQLNRLLGVSWRLM |
| | QIQPNREDTETLIKAVYTLYQQRGLILPVRTLLLK |
| | FFSKIYQTEELRSCRFRYRSKVLSRWLAGLPLQLA |
| | HLGSRNPELSTQLIDIIHTAAARANKELLKSLQAT |
| | ALRIYDPQEGAVVVLPADSQQRLVQLVYFLPSLPA |
| | DLLSRLSRCCIMGRLSSSLAAMLIGILHMRSSFSG |
| | WKYSAKDWLMSDVDYFSFLFSTLTGFSKEELTWLQ |
| | SLRGVPHVIQTQLSPVLLYLTDLDQFLHHWDVTEA |
| | VFHSLLVIPARSQNFDILQSAISKHLVGLTVIPDS |
| | TAGCVFGVICKLLDHTCVVSETLLPFLASCCYSLL |
| | YFLLTIEKGEAEHLRKRDKLWGVCVSILALLPRVL |
| | RLMLQSLRVNRVGPEELPVVGQLLRLLLQHAPLRT |
| | HMLTNAILVQQIIKNITTLKSGSVQEQWLTDLHYC |
| | ENVYITGHPQGPSALATVY |
| 13 | ATGTCGAGAATGACTAAAAAAAGAAAACGCCAACA |
| | TGATTTTCAAAAAGTAAAATTGAAAGTTGGTAAAA |
| | AGAAGCCCAAGTTACAAAATGCTACTCCTACAAAC |
| | TTTAAAACAAAGACTATACATCTGCCTGAGCAACT |
| | CAAAGAGGATGGAACACTTCCAACAAACAATAGAA |
| | AACTTAACATAAAGGATTTGCTGTCACAGATGCAT |
| | CACTACAATGCTGGGGTTAAACAAAGTGCTCTTCT |
| | TGGACTTAAAGACCTTTTGTCTCAATACCCATTTA |
| | TAATTGATGCACACCTTTCAAACATATTAAGTGAA |
| | GTGACTGCTGTGTTTACAGATAAAGATGCTAATGT |
| | ACGATTAGCAGCAGTTCAACTTCTTCAATTCCTGG |
| | CCCCCAAAATACGAGCTGAACAAATTTCTCCATTT |
| | TTTCCTTTGGTAAGTGCCCATCTCTCTAGTGCCAT |
| | GACTCACATTACTGAAGGAATTCAGGAGGACTCTT |
| | TAAAAGTTTTGGACATTCTGCTGGAACAGTACCCA |
| | GCTCTAATTACTGGCCGTAGCAGCATATTGCTTAA |
| | GAATTTTGTAGAACTTATTTCTCATCAGCAGCTGT |
| | CCAAAGGACTGATAAATAGAGACAGATCCCAGTCC |
| | TGGATACTTTCTGTAAATCCTAATCGGAGACTCAC |
| | TTCTCAGCAATGGAGGCTGAAAGTCTTAGTGAGAC |
| | TCAGTAAATTCCTTCAGGCCTTGGCAGATGGATCC |
| | AGTAGGTTGAGAGAAAGTGAAGGACTTCAGGAACA |
| | GAAAGAAAATCCCCATGCCACTAGCAACTCCATTT |
| | TTATCAACTGGAAGGAACATGCCAACGACCAGCAA |
| | CACATCCAGGTTTATGAAAATGGGGGTTCACAGCC |
| | AAATGTCAGTTCACAGTTCAGGCTACGGTATCTGG |
| | TTGGAGGACTGAGTGGTGTGGATGAAGGCCTGTCA |
| | TCTACTGAAAACCTGAAAGGATTTATTGAGATAAT |
| | AATTCCATTGCTAATTGAATGCTGGGTTGAAGCTG |
| | TACCTCCACAACTAGCTACTCCTGTTGGGAATGGT |
| | ATAGAACGAGAACCTCTACAGGTTATGCAGCAAGT |
| | TCTTAATATTATTTCCCTTCTGTGGAAACTCTCTA |
| | AACAACAGGATGAAACCCATAAATTGGAGTCATGG |
| | CTTCGAAAGAACTACCTTATTGATTTTAAACACCA |
| | TTTTATGAGTCGTTTTCCATATGTCTTAAAAGAAA |
| | TAACCAAGCACAAAAGGAAAGAGCCAAATAAAAGC |
| | ATCAAGCATTGCACAGTTCTCTCCAATAACATAGA |
| | TCATCTCTTACTGAATTTAACACTGTCTGATATCA |
| | TGGTCTCCCTGGCAAATGCGTCAACTTTGCAGAAG |
| | GATTGCAGTTGGATAGAAATGATAAGGAAATTTGT |
| | AACAGAGACCCTTGAAGATGGCTCTAGGCTAAATA |
| | GTAAGCAACTGAACAGATTGCTGGGAGTATCCTGG |
| | AGGTTAATGCAAATACAGCCAAACAGAGAGGACAC |
| | AGAGACTCTTATTAAGGCAGTTTATACATTATATC |
| | AGCAGAGGGGCCTTATCCTTCCAGTTCGGACTTTG |
| | TTATTGAAGTTTTTCAGTAAAATCTATCAGACAGA |
| | AGAACTGAGATCTTGTAGATTCAGATATCGTAGTA |
| | AAGTGTTATCCCGTTGGCTGGCTGGCTTACCATTG |
| | CAACTTGCTCATCTTGGCTCCCGAAATCCTGAGCT |
| | CTCTACACAGCTTATCGATATCATTCATACCGCTG |
| | CAGCACGAGCAAATAAAGAATTACTAAAAAGTTTA |
| | CAAGCTACTGCCCTCCGAATTTATGATCCACAAGA |
| | AGGTGCTGTGGTGGTTCTCCCTGCAGACTCTCAGC |
| | AGCGTTTGGTTCAGCTTGTATATTTCCTACCCAGT |
| | CTGCCGGCTGATTTGCTTTCTCGGTTAAGTCGTTG |
| | CTGTATTATGGGAAGACTCAGTTCAAGTTTGGCTG |
| | CCATGCTTATCGGGATACTGCACATGAGATCATCA |
| | TTTTCTGGGTGGAAGTATTCAGCTAAAGACTGGTT |
| | GATGAGTGATGTAGACTATTTCAGCTTCTTATTTT |
| | CCACACTTACAGGGTTTTCGAAAGAGGAGTTGACT |
| | TGGCTTCAGAGCCTTCGAGGAGTTCCTCATGTCAT |
| | CCAGACACAGCTTTCCCCTGTGCTTCTCTACCTTA |
| | CAGATTTGGATCAATTTTTACACCACTGGGATGTA |
| | ACAGAGGCAGTTTTTCACAGTTTATTGGTTATTCC |
| | TGCCCGAAGTCAGAACTTTGACATCTTGCAAAGTG |
| | CCATCAGTAAGCATTTGGTTGGGTTGACTGTAATT |
| | CCTGACAGCACGGCTGGCTGTGTTTTTGGTGTTAT |
| | CTGTAAGCTCCTGGATCATACTTGTGTAGTTAGTG |
| | AGACTCTACTGCCATTTCTGGCTTCTTGTTGCTAC |
| | AGTCTTCTTTATTTTCTGCTCACTATAGAGAAAGG |
| | GGAAGCAGAACATCTAAGAAAGAGGTTGATGCTGC |
| | AGAGCCTGCGGGTGAACAGAGTTGGGCCTGAGGAG |
| | CTGCCTGTTGTGGGCCAGCTGCTTCGACTGCTGCT |
| | TCAGCATGCACCCCTCAGGACTCATATGTTGACCA |
| | ATGCGATCTTGGTGCAGCAGATCATCAAGAATATC |
| | ACGACATTGAAGAGTGGAAGTGTTCAGGAACAGTG |
| | GCTCACAGACTTACATTACTGCTTCAACGTGTATA |
| | TCACTGGGCATCCCCAAGGGCCCAGTGCACTGGCT |
| | ACAGTGTATTGA |
| 14 | MSRMTKKRKRQHDFQKVKLKVGKKKPKLQNATPTN |
| | FKTKTIHLPEQLKEDGTLPTNNRKLNIKDLLSQMH |
| | HYNAGVKQSALLGLKDLLSQYPFIIDAHLSNILSE |
| | VTAVFTDKDANVRLAAVQLLQFLAPKIRAEQISPF |
| | FPLVSAHLSSAMTHITEGIQEDSLKVLDILLEQYP |
| | ALITGRSSILLKNEVELISHQQLSKGLINRDRSQS |
| | WILSVNPNRRLTSQQWRLKVLVRLSKFLQALADGS |
| | SRLRESEGLQEQKENPHATSNSIFINWKEHANDQQ |
| | HIQVYENGGSQPNVSSQFRLRYLVGGLSGVDEGLS |
| | STENLKGFIEIIIPLLIECWVEAVPPQLATPVGNG |
| | IEREPLQVMQQVLNIISLLWKLSKQQDETHKLESW |
| | LRKNYLIDFKHHEMSRFPYVLKEITKHKRKEPNKS |
| | IKHCTVLSNNIDHLLLNLTLSDIMVSLANASTLQK |
| | DCSWIEMIRKFVTETLEDGSRLNSKQLNRLLGVSW |
| | RLMQIQPNREDTETLIKAVYTLYQQRGLILPVRTL |
| | LLKFFSKIYQTEELRSCRFRYRSKVLSRWLAGLPL |
| | QLAHLGSRNPELSTQLIDIIHTAAARANKELLKSL |
| | QATALRIYDPQEGAVVVLPADSQQRLVQLVYFLPS |
| | LPADLLSRLSRCCIMGRLSSSLAAMLIGILHMRSS |
| | FSGWKYSAKDWLMSDVDYFSFLFSTLTGFSKEELT |
| | WLQSLRGVPHVIQTQLSPVLLYLTDLDQFLHHWDV |
| | TEAVEHSLLVIPARSQNEDILQSAISKHLVGLTVI |
| | PDSTAGCVFGVICKLLDHTCVVSETLLPFLASCCY |
| | SLLYFLLTIEKGEAEHLRKRLMLQSLRVNRVGPEE |
| | LPVVGQLLRLLLQHAPLRTHMLTNAILVQQIIKNI |
| | TTLKSGSVQEQWLTDLHYCENVYITGHPQGPSALA |
| | TVY |
| 15 | ATGGCACGGTTCGGCTTGCCCGCGCTTCTCTGCAC |
| | CCTGGCAGTGCTCAGCGCCGCGCTGCTGGCTGCCG |
| | AGCTCAAGTCGAAAAGTTGCTCGGAAGTGCGACGT |
| | CTTTACGTGTCCAAAGGCTTCAACAAGAACGATGC |
| | CCCCCTCCACGAGATCAACGGTGATCATTTGAAGA |
| | TCTGTCCCCAGGGTTCTACCTGCTGCTCTCAAGAG |
| | ATGGAGGAGAAGTACAGCCTGCAAAGTAAAGATGA |
| | TTTCAAAAGTGTGGTCAGCGAACAGTGCAATCATT |
| | TGCAAGCTGTCTTTGCTTCACGTTACAAGAAGTTT |
| | GATGAATTCTTCAAAGAACTACTTGAAAATGCAGA |
| | GAAATCCCTGAATGATATGTTTGTGAAGACATATG |
| | GCCATTTATACATGCAAAATTCTGAGCTATTTAAA |
| | GATCTCTTCGTAGAGTTGAAACGTTACTACGTGGT |
| | GGGAAATGTGAACCTGGAAGAAATGCTAAATGACT |
| | TCTGGGCTCGCCTCCTGGAGCGGATGTTCCGCCTG |
| | GTGAACTCCCAGTACCACTTTACAGATGAGTATCT |
| | GGAATGTGTGAGCAAGTATACGGAGCAGCTGAAGC |
| | CCTTCGGAGATGTCCCTCGCAAATTGAAGCTCCAG |
| | GTTACTCGTGCTTTTGTAGCAGCCCGTACTTTCGC |
| | TCAAGGCTTAGCGGTTGCGGGAGATGTCGTGAGCA |
| | AGGTCTCCGTGGTAAACCCCACAGCCCAGTGTACC |
| | CATGCCCTGTTGAAGATGATCTACTGCTCCCACTG |
| | CCGGGGTCTCGTGACTGTGAAGCCATGTTACAACT |
| | ACTGCTCAAACATCATGAGAGGCTGTTTGGCCAAC |
| | CAAGGGGATCTCGATTTTGAATGGAACAATTTCAT |
| | AGATGCTATGCTGATGGTGGCAGAGAGGCTAGAGG |
| | GTCCTTTCAACATTGAATCGGTCATGGATCCCATC |
| | GATGTGAAGATTTCTGATGCTATTATGAACATGCA |
| | GGATAATAGTGTTCAAGTGTCTCAGAAGGTTTTCC |
| | AGGGATGTGGACCCCCCAAGCCCCTCCCAGCTGGA |
| | CGAATTTCTCGTTCCATCTCTGAAAGTGCCTTCAG |
| | TGCTCGCTTCAGACCACATCACCCCGAGGAACGCC |
| | CAACCACAGCAGCTGGCACTAGTTTGGACCGACTG |
| | GTTACTGATGTCAAGGAGAAACTGAAACAGGCCAA |
| | GAAATTCTGGTCCTCCCTTCCGAGCAACGTTTGCA |
| | ACGATGAGAGGATGGCTGCAGGAAACGGCAATGAG |
| | GATGACTGTTGGAATGGGAAAGGCAAAAGCAGGTA |
| | CCTGTTTGCAGTGACAGGAAATGGATTAGCCAACC |
| | AGGGCAACAACCCAGAGGTCCAGGTTGACACCAGC |
| | AAACCAGACATACTGATCCTTCGTCAAATCATGGC |
| | TCTTCGAGTGATGACCAGCAAGATGAAGAATGCAT |
| | ACAATGGGAACGACGTGGACTTCTTTGATATCAGT |
| | GATGAAAGTAGTGGAGAAGGAAGTGGAAGTGGCTG |
| | TGAGTATCAGCAGTGCCCTTCAGAGTTTGACTACA |
| | ATGCCACTGACCATGCTGGGAAGAGTGCCAATGAG |
| | AAAGCCGACAGTGCTGGTGTCCGTCCTGGGGCACA |
| | GGCCTACCTCCTCACTGTCTTCTGCATCTTGTTCC |
| | TGGTTATGCAGAGAGAGTGGAGATAA |
| 16 | MARFGLPALLCTLAVLSAALLAAELKSKSCSEVRR |
| | LYVSKGENKNDAPLHEINGDHLKICPQGSTCCSQE |
| | MEEKYSLQSKDDFKSVVSEQCNHLQAVFASRYKKE |
| | DEFFKELLENAEKSLNDMFVKTYGHLYMQNSELFK |
| | DLFVELKRYYVVGNVNLEEMLNDFWARLLERMFRL |
| | VNSQYHFTDEYLECVSKYTEQLKPFGDVPRKLKLQ |
| | VTRAFVAARTFAQGLAVAGDVVSKVSVVNPTAQCT |
| | HALLKMIYCSHCRGLVTVKPCYNYCSNIMRGCLAN |
| | QGDLDFEWNNFIDAMLMVAERLEGPFNIESVMDPI |
| | DVKISDAIMNMQDNSVQVSQKVFQGCGPPKPLPAG |
| | RISRSISESAFSARFRPHHPEERPTTAAGTSLDRL |
| | VTDVKEKLKQAKKEWSSLPSNVCNDERMAAGNGNE |
| | DDCWNGKGKSRYLFAVTGNGLANQGNNPEVQVDTS |
| | KPDILILRQIMALRVMTSKMKNAYNGNDVDFFDIS |
| | DESSGEGSGSGCEYQQCPSEFDYNATDHAGKSANE |
| | KADSAGVRPGAQAYLLTVFCILFLVMQREWR |
| 17 | ALLAAELKSKSCSEVRRLYVSKGENKNDAPLHEIN |
| | GDHLKICPQGSTCCSQEMEEKYSLQSKDDFKSVVS |
| | EQCNHLQAVFASRYKKEDEFFKELLENAEKSLNDM |
| | FVKTYGHLYMQNSELFKDLFVELKRYYVVGNVNLE |
| | EMLNDFWARLLERMFRLVNSQYHFTDEYLECVSKY |
| | TEQLKPFGDVPRKLKLQVTRAFVAARTFAQGLAVA |
| | GDVVSKVSVVNPTAQCTHALLKMIYCSHCRGLVTV |
| | KPCYNYCSNIMRGCLANQGDLDFEWNNFIDAMLMV |
| | AERLEGPFNIESVMDPIDVKISDAIMNMQDNSVQV |
| | SQKVFQGCGPPKPLPAGRISRSISESAFSARFRPH |
| | HPEERPTTAAGTSLDRLVTDVKEKLKQAKKFWSSL |
| | PSNVCNDERMAAGNGNEDDCWNGKGKSRYLFAVTG |
| | NGLANQGNNPEVQVDTSKPDILILRQIMALRVMTS |
| | KMKNAYNGNDVDFFDISDESSGEGSGSGCEYQQCP |
| | SEFDYNATDHAGKSANEKADS |
| 18 | MWPLVAALLLGSACCGSAQLLFNKTKSVEFTFCND |
| | TVVIPCFVTNMEAQNTTEVYVKWKFKGRDIYTFDG |
| | ALNKSTVPTDESSAKIEVSQLLKGDASLKMDKSDA |
| | VSHTGNYTCEVTELTREGETIIELKYRVVSWFSPN |
| | ENILIVIFPIFAILLFWGQFGIKTLKYRSGGMDEK |
| | TIALLVAGLVITVIVIVGAILFVPGEYSLKNATGL |
| | GLIVTSTGILILLHYYVESTAIGLTSFVIAILVIQ |
| | VIAYILAVVGLSLCIAACIPMHGPLLISGLSILAL |
| | AQLLGLVYMKFVASNQKTIQPPRKAVEEPLNAFKE |
| | SKGMMNDE |
| 19 | MWPLVAALLLGSACCGSAQLLENKTKSVEFTFCND |
| | TVVIPCFVTNMEAQNTTEVYVKWKFKGRDIYTFDG |
| | ALNKSTVPTDESSAKIEVSQLLKGDASLKMDKSDA |
| | VSHTGNYTCEVTELTREGETIIELKYRVVSWFSPN |
| | ENILIVIFPIFAILLEWGQFGIKTLKYRSGGMDEK |
| | TIALLVAGLVITVIVIVGAILFVPGEYSLKNATGL |
| | GLIVISTGILILLHYYVESTAIGLTSFVIAILVIQ |
| | VIAYILAVVGLSLCIAACIPMHGPLLISGLSILAL |
| | AQLLGLVYMKFVASNQKTIQPPRNN |
| 20 | MWPLVAALLLGSACCGSAQLLFNKTKSVEFTFCND |
| | TVVIPCFVTNMEAQNTTEVYVKWKFKGRDIYTFDG |
| | ALNKSTVPTDESSAKIEVSQLLKGDASLKMDKSDA |
| | VSHTGNYTCEVTELTREGETIIELKYRVVSWESPN |
| | ENILIVIFPIFAILLFWGQFGIKTLKYRSGGMDEK |
| | TIALLVAGLVITVIVIVGAILFVPGEYSLKNATGL |
| | GLIVTSTGILILLHYYVFSTAIGLTSFVIAILVIQ |
| | VIAYILAVVGLSLCIAACIPMHGPLLISGLSILAL |
| | AQLLGLVYMKFVASNQKTIQPPRKAVEEPLNE |
| 21 | MWPLVAALLLGSACCGSAQLLENKTKSVEFTFCND |
| | TVVIPCFVTNMEAQNTTEVYVKWKFKGRDIYTFDG |
| | ALNKSTVPTDESSAKIEVSQLLKGDASLKMDKSDA |
| | VSHTGNYTCEVTELTREGETIIELKYRVVSWESPN |
| | ENILIVIFPIFAILLFWGQFGIKTLKYRSGGMDEK |
| | TIALLVAGLVITVIVIVGAILFVPGEYSLKNATGL |
| | GLIVTSTGILILLHYYVFSTAIGLTSFVIAILVIQ |
| | VIAYILAVVGLSLCIAACIPMHGPLLISGLSILAL |
| | AQLLGLVYMKFVE |
| 22 | QLLFNKTKSVEFTFCNDTVVIPCFVTNMEAQNTTE |
| | VYVKWKFKGRDIYTEDGALNKSTVPTDESSAKIEV |
| | SQLLKGDASLKMDKSDAVSHTGNYTCEVTELTREG |
| | ETIIELKYRVVSWFSPNENILIVIFPIFAILLFWG |
| | QFGIKTLKYRSGGMDEKTIALLVAGLVITVIVIVG |
| | AILFVPGEYSLKNATGLGLIVTSTGILILLHYYVF |
| | STAIGLTSFVIAILVIQVIAYILAVVGLSLCIAAC |
| | IPMHGPLLISGLSILALAQLLGLVYMKEVASNQKT |
| | IQPPRKAVEEPLNAFKESKGMMNDE |
| 23 | QLLFNKTKSVEFTFCNDTVVIPCFVTNMEAQNTTE |
| | VYVKWKFKGRDIYTFDGALNKSTVPTDESSAKIEV |
| | SQLLKGDASLKMDKSDAVSHTGNYTCEVTELTREG |
| | ETIIELKYRVVSWESPNENILIVIFPIFAILLFWG |
| | QFGIKTLKYRSGGMDEKTIALLVAGLVITVIVIVG |
| | AILFVPGEYSLKNATGLGLIVTSTGILILLHYYVE |
| | STAIGLTSFVIAILVIQVIAYILAVVGLSLCIAAC |
| | IPMHGPLLISGLSILALAQLLGLVYMKFVASNQKT |
| | IQPPRNN |
| 24 | QLLFNKTKSVEFTFCNDTVVIPCFVTNMEAQNTTE |
| | VYVKWKFKGRDIYTFDGALNKSTVPTDESSAKIEV |
| | SQLLKGDASLKMDKSDAVSHTGNYTCEVTELTREG |
| | ETIIELKYRVVSWFSPNENILIVIFPIFAILLFWG |
| | QFGIKTLKYRSGGMDEKTIALLVAGLVITVIVIVG |
| | AILFVPGEYSLKNATGLGLIVTSTGILILLHYYVF |
| | STAIGLTSEVIAILVIQVIAYILAVVGLSLCIAAC |
| | IPMHGPLLISGLSILALAQLLGLVYMKFVASNQKT |
| | IQPPRKAVEEPLNE |
| 25 | QLLFNKTKSVEFTFCNDTVVIPCFVTNMEAQNTTE |
| | VYVKWKFKGRDIYTFDGALNKSTVPTDESSAKIEV |
| | SQLLKGDASLKMDKSDAVSHTGNYTCEVTELTREG |
| | ETIIELKYRVVSWFSPNENILIVIFPIFAILLFWG |
| | QFGIKTLKYRSGGMDEKTIALLVAGLVITVIVIVG |
| | AILFVPGEYSLKNATGLGLIVTSTGILILLHYYVE |
| | STAIGLTSFVIAILVIQVIAYILAVVGLSLCIAAC |
| | IPMHGPLLISGLSILALAQLLGLVYMKEVE26MGR |
| | AMVARLGLGLLLLALLLPTQIYSSETTTGTSSNSS |
| | QSTSNSGLAPNPTNATTKAAGGALQSTASLFVVSL |
| | SLLHLYS |
| 27 | SETTTGTSSNSSQSTSNSGLAPNPTNATTKAAG |
| 28 | MVGRFCPESPPGFVRVAATSAVSLDPPSGEPRPGC |
| | GYPGPRSAASRVYGCTAPARETGGWAWETLAGAGA |
| | KKIYSSETTTGTSSNSSQSTSNSGLAPNPTNATTK |
| | AAGGALQSTASLFVVSLSLLHLYS |
| 29 | MTVARPSVPAALPLLGELPRLLLLVLLCLPAVWGD |
| | CGLPPDVPNAQPALEGRTSFPEDTVITYKCEESFV |
| | KIPGEKDSVICLKGSQWSDIEEFCNRSCEVPTRLN |
| | SASLKQPYITQNYFPVGTVVEYECRPGYRREPSLS |
| | PKLTCLQNLKWSTAVEFCKKKSCPNPGEIRNGQID |
| | VPGGILFGATISFSCNTGYKLFGSTSSFCLISGSS |
| | VQWSDPLPECREIYCPAPPQIDNGIIQGERDHYGY |
| | RQSVTYACNKGFTMIGEHSIYCTVNNDEGEWSGPP |
| | PECRGKSLTSKVPPTVQKPTTVNVPTTEVSPTSQK |
| | TTTKTTTPNAQATRSTPVSRTTKHFHETTPNKGSG |
| | TTSGTTRLLSGHTCFTLTGLLGTLVTMGLLT |
| 30 | DCGLPPDVPNAQPALEGRTSFPEDTVITYKCEESF |
| | VKIPGEKDSVICLKGSQWSDIEEFCNRSCEVPTRL |
| | NSASLKQPYITQNYFPVGTVVEYECRPGYRREPSL |
| | SPKLTCLQNLKWSTAVEFCKKKSCPNPGEIRNGQI |
| | DVPGGILFGATISFSCNTGYKLFGSTSSFCLISGS |
| | SVQWSDPLPECREIYCPAPPQIDNGIIQGERDHYG |
| | YRQSVTYACNKGFTMIGEHSIYCTVNNDEGEWSGP |
| | PPECRGKSLTSKVPPTVQKPTTVNVPTTEVSPTSQ |
| | KTTTKTTTPNAQATRSTPVSRTTKHFHETTPNKGS |
| | GTTS |
| 31 | MGIQGGSVLFGLLLVLAVFCHSGHSLQCYNCPNPT |
| | ADCKTAVNCSSDEDACLITKAGLQVYNKCWKFEHC |
| | NFNDVTTRLRENELTYYCCKKDLCNFNEQLENGGT |
| | SLSEKTVLLLVTPFLAAAWSLHP |
| 32 | LQCYNCPNPTADCKTAVNCSSDEDACLITKAGLQV |
| | YNKCWKFEHCNENDVTTRLRENELTYYCCKKDLCN |
| | ENEQLEN |
| 33 | MEPPGRRECPFPSWRFPGLLLAAMVLLLYSFSDAC |
| | EEPPTFEAMELIGKPKPYYEIGERVDYKCKKGYFY |
| | IPPLATHTICDRNHTWLPVSDDACYRETCPYIRDP |
| | LNGQAVPANGTYEFGYQMHFICNEGYYLIGEEILY |
| | CELKGSVAIWSGKPPICEKVLCTPPPKIKNGKHTF |
| | SEVEVFEYLDAVTYSCDPAPGPDPFSLIGESTIYC |
| | GDNSVWSRAAPECKVVKCRFPVVENGKQISGFGKK |
| | FYYKATVMFECDKGFYLDGSDTIVCDSNSTWDPPV |
| | PKCLKVLPPSSTKPPALSHSVSTSSTTKSPASSAS |
| | GPRPTYKPPVSNYPGYPKPEEGILDSLDVWVIAVI |
| | VIAIVVGVAVICVVPYRYLQRRKKKGTYLTDETHR |
| | EVKFTSL |
| 34 | CEEPPTFEAMELIGKPKPYYEIGERVDYKCKKGYF |
| | YIPPLATHTICDRNHTWLPVSDDACYRETCPYIRD |
| | PLNGQAVPANGTYEFGYQMHFICNEGYYLIGEEIL |
| | YCELKGSVAIWSGKPPICEKVLCTPPPKIKNGKHT |
| | FSEVEVFEYLDAVTYSCDPAPGPDPFSLIGESTIY |
| | CGDNSVWSRAAPECKVVKCRFPVVENGKQISGFGK |
| | KFYYKATVMFECDKGFYLDGSDTIVCDSNSTWDPP |
| | VPKCLKVLPPSSTKPPALSHSVSTSSTTKSPASSA |
| | SGPRPTYKPPVSNYPGYPKPEEGILDSLDVWVIAV |
| | IVIAIVVGVAVICVVPYRYLQRRKKKGTYLTDETH |
| | REVKFTSL |
| 35 | MVVMAPRTLFLLLSGALTLTETWAGSHSMRYFSAA |
| | VSRPGRGEPRFIAMGYVDDTQFVRFDSDSACPRME |
| | PRAPWVEQEGPEYWEEETRNTKAHAQTDRMNLQTL |
| | RGYYNQSEASSHTLQWMIGCDLGSDGRLLRGYEQY |
| | AYDGKDYLALNEDLRSWTAADTAAQISKRKCEAAN |
| | VAEQRRAYLEGTCVEWLHRYLENGKEMLQRADPPK |
| | THVTHHPVEDYEATLRCWALGFYPAEIILTWQRDG |
| | EDQTQDVELVETRPAGDGTFQKWAAVVVPSGEEQR |
| | YTCHVQHEGLPEPLMLRWKQSSLPTIPIMGIVAGL |
| | VVLAAVVTGAAVAAVLWRKKSSD |
| 36 | GSHSMRYFSAAVSRPGRGEPRFIAMGYVDDTQFVR |
| | EDSDSACPRMEPRAPWVEQEGPEYWEEETRNTKAH |
| | AQTDRMNLQTLRGYYNQSEASSHTLQWMIGCDLGS |
| | DGRLLRGYEQYAYDGKDYLALNEDLRSWTAADTAA |
| | QISKRKCEAANVAEQRRAYLEGTCVEWLHRYLENG |
| | KEMLQRADPPKTHVTHHPVEDYEATLRCWALGFYP |
| | AEIILTWQRDGEDQTQDVELVETRPAGDGTFQKWA |
| | AVVVPSGEEQRYTCHVQHEGLPEPLMLRWKQSSLP |
| | TIPIMGIVAGLVVLAAVVTGAAVAAVLWRKKSSD |
| 37 | MVDGTLLLLLSEALALTQTWAGSHSLKYFHTSVSR |
| | PGRGEPRFISVGYVDDTQFVRFDNDAASPRMVPRA |
| | PWMEQEGSEYWDRETRSARDTAQIFRVNLRTLRGY |
| | YNQSEAGSHTLQWMHGCELGPDGRFLRGYEQFAYD |
| | GKDYLTLNEDLRSWTAVDTAAQISEQKSNDASEAE |
| | HQRAYLEDTCVEWLHKYLEKGKETLLHLEPPKTHV |
| | THHPISDHEATLRCWALGFYPAEITLTWQQDGEGH |
| | TQDTELVETRPAGDGTFQKWAAVVVPSGEEQRYTC |
| | HVQHEGLPEPVTLRWKPASQPTIPIVGIIAGLVLL |
| | GSVVSGAVVAAVIWRKKSSGGKGGSYSKAEWSDSA |
| | QGSESHSL |
| 38 | GSHSLKYFHTSVSRPGRGEPRFISVGYVDDTQFVR |
| | EDNDAASPRMVPRAPWMEQEGSEYWDRETRSARDT |
| | AQIFRVNLRTLRGYYNQSEAGSHTLQWMHGCELGP |
| | DGRFLRGYEQFAYDGKDYLTLNEDLRSWTAVDTAA |
| | QISEQKSNDASEAEHQRAYLEDTCVEWLHKYLEKG |
| | KETLLHLEPPKTHVTHHPISDHEATLRCWALGFYP |
| | AEITLTWQQDGEGHTQDTELVETRPAGDGTFQKWA |
| | AVVVPSGEEQRYTCHVQHEGLPEPVTLRWKPASQP |
| | TIPIVGIIAGLVLLGSVVSGAVVAAVIWRKKSSGG |
| | KGGSYSKAEWSDSAQGSESHSL |
| 39 | MGASSPRSPEPVGPPAPGLPFCCGGSLLAVVVLLA |
| | LPVAWGQCNAPEWLPFARPTNLTDEFEFPIGTYLN |
| | YECRPGYSGRPFSIICLKNSVWTGAKDRCRRKSCR |
| | NPPDPVNGMVHVIKGIQFGSQIKYSCTKGYRLIGS |
| | SSATCIISGDTVIWDNETPICDRIPCGLPPTITNG |
| | DFISTNRENFHYGSVVTYRCNPGSGGRKVFELVGE |
| | PSIYCTSNDDQVGIWSGPAPQCIIPNKCTPPNVEN |
| | GILVSDNRSLFSLNEVVEFRCQPGFVMKGPRRVKC |
| | QALNKWEPELPSCSRVCQPPPDVLHAERTQRDKDN |
| | ESPGQEVFYSCEPGYDLRGAASMRCTPQGDWSPAA |
| | PTCEVKSCDDFMGQLLNGRVLFPVNLQLGAKVDFV |
| | CDEGFQLKGSSASYCVLAGMESLWNSSVPVCEQIF |
| | CPSPPVIPNGRHTGKPLEVFPFGKTVNYTCDPHPD |
| | RGTSFDLIGESTIRCTSDPQGNGVWSSPAPRCGIL |
| | GHCQAPDHFLFAKLKTQTNASDEPIGTSLKYECRP |
| | EYYGRPFSITCLDNLVWSSPKDVCKRKSCKTPPDP |
| | VNGMVHVITDIQVGSRINYSCTTGHRLIGHSSAEC |
| | ILSGNAAHWSTKPPICQRIPCGLPPTIANGDFIST |
| | NRENFHYGSVVTYRCNPGSGGRKVFELVGEPSIYC |
| | TSNDDQVGIWSGPAPQCIIPNKCTPPNVENGILVS |
| | DNRSLFSLNEVVEFRCQPGFVMKGPRRVKCQALNK |
| | WEPELPSCSRVCQPPPDVLHAERTQRDKDNFSPGQ |
| | EVFYSCEPGYDLRGAASMRCTPQGDWSPAAPTCEV |
| | KSCDDFMGQLLNGRVLFPVNLQLGAKVDFVCDEGF |
| | QLKGSSASYCVLAGMESLWNSSVPVCEQIFCPSPP |
| | VIPNGRHTGKPLEVFPFGKAVNYTCDPHPDRGTSF |
| | DLIGESTIRCTSDPQGNGVWSSPAPRCGILGHCQA |
| | PDHFLFAKLKTQTNASDEPIGTSLKYECRPEYYGR |
| | PFSITCLDNLVWSSPKDVCKRKSCKTPPDPVNGMV |
| | HVITDIQVGSRINYSCTTGHRLIGHSSAECILSGN |
| | TAHWSTKPPICQRIPCGLPPTIANGDFISTNRENF |
| | HYGSVVTYRCNLGSRGRKVFELVGEPSIYCTSNDD |
| | QVGIWSGPAPQCIIPNKCTPPNVENGILVSDNRSL |
| | ESLNEVVEFRCQPGFVMKGPRRVKCQALNKWEPEL |
| | PSCSRVCQPPPEILHGEHTPSHQDNFSPGQEVFYS |
| | CEPGYDLRGAASLHCTPQGDWSPEAPRCAVKSCDD |
| | FLGQLPHGRVLFPLNLQLGAKVSFVCDEGFRLKGS |
| | SVSHCVLVGMRSLWNNSVPVCEHIFCPNPPAILNG |
| | RHTGTPSGDIPYGKEISYTCDPHPDRGMTENLIGE |
| | STIRCTSDPHGNGVWSSPAPRCELSVRAGHCKTPE |
| | QFPFASPTIPINDFEFPVGTSLNYECRPGYFGKMF |
| | SISCLENLVWSSVEDNCRRKSCGPPPEPENGMVHI |
| | NTDTQFGSTVNYSCNEGFRLIGSPSTTCLVSGNNV |
| | TWDKKAPICEIISCEPPPTISNGDFYSNNRTSFHN |
| | GTVVTYQCHTGPDGEQLFELVGERSIYCTSKDDQV |
| | GVWSSPPPRCISTNKCTAPEVENAIRVPGNRSFFT |
| | LTEIIRFRCQPGFVMVGSHTVQCQTNGRWGPKLPH |
| | CSRVCQPPPEILHGEHTLSHQDNESPGQEVFYSCE |
| | PSYDLRGAASLHCTPQGDWSPEAPRCTVKSCDDFL |
| | GQLPHGRVLLPLNLQLGAKVSFVCDEGFRLKGRSA |
| | SHCVLAGMKALWNSSVPVCEQIFCPNPPAILNGRH |
| | TGTPFGDIPYGKEISYACDTHPDRGMTFNLIGESS |
| | IRCTSDPQGNGVWSSPAPRCELSVPAACPHPPKIQ |
| | NGHYIGGHVSLYLPGMTISYICDPGYLLVGKGFIF |
| | CTDQGIWSQLDHYCKEVNCSFPLEMNGISKELEMK |
| | KVYHYGDYVTLKCEDGYTLEGSPWSQCQADDRWDP |
| | PLAKCTSRTHDALIVGTLSGTIFFILLIIFLSWII |
| | LKHRKGNNAHENPKEVAIHLHSQGGSSVHPRTLQT |
| | NEENSRVLP |
| 40 | QCNAPEWLPFARPTNLTDEFEFPIGTYLNYECRPG |
| | YSGRPFSIICLKNSVWTGAKDRCRRKSCRNPPDPV |
| | NGMVHVIKGIQFGSQIKYSCTKGYRLIGSSSATCI |
| | ISGDTVIWDNETPICDRIPCGLPPTITNGDFISTN |
| | RENFHYGSVVTYRCNPGSGGRKVFELVGEPSIYCT |
| | SNDDQVGIWSGPAPQCIIPNKCTPPNVENGILVSD |
| | NRSLESLNEVVEFRCQPGFVMKGPRRVKCQALNKW |
| | EPELPSCSRVCQPPPDVLHAERTQRDKDNFSPGQE |
| | VFYSCEPGYDLRGAASMRCTPQGDWSPAAPTCEVK |
| | SCDDEMGQLLNGRVLFPVNLQLGAKVDFVCDEGFQ |
| | LKGSSASYCVLAGMESLWNSSVPVCEQIFCPSPPV |
| | IPNGRHTGKPLEVEPFGKTVNYTCDPHPDRGTSFD |
| | LIGESTIRCTSDPQGNGVWSSPAPRCGILGHCQAP |
| | DHFLFAKLKTQTNASDFPIGTSLKYECRPEYYGRP |
| | FSITCLDNLVWSSPKDVCKRKSCKTPPDPVNGMVH |
| | VITDIQVGSRINYSCTTGHRLIGHSSAECILSGNA |
| | AHWSTKPPICQRIPCGLPPTIANGDFISTNRENFH |
| | YGSVVTYRCNPGSGGRKVFELVGEPSIYCTSNDDQ |
| | VGIWSGPAPQCIIPNKCTPPNVENGILVSDNRSLF |
| | SLNEVVEFRCQPGFVMKGPRRVKCQALNKWEPELP |
| | SCSRVCQPPPDVLHAERTQRDKDNFSPGQEVFYSC |
| | EPGYDLRGAASMRCTPQGDWSPAAPTCEVKSCDDE |
| | MGQLLNGRVLFPVNLQLGAKVDFVCDEGFQLKGSS |
| | ASYCVLAGMESLWNSSVPVCEQIFCPSPPVIPNGR |
| | HTGKPLEVEPFGKAVNYTCDPHPDRGTSFDLIGES |
| | TIRCTSDPQGNGVWSSPAPRCGILGHCQAPDHELF |
| | AKLKTQTNASDFPIGTSLKYECRPEYYGRPFSITC |
| | LDNLVWSSPKDVCKRKSCKTPPDPVNGMVHVITDI |
| | QVGSRINYSCTTGHRLIGHSSAECILSGNTAHWST |
| | KPPICQRIPCGLPPTIANGDFISTNRENFHYGSVV |
| | TYRCNLGSRGRKVFELVGEPSIYCTSNDDQVGIWS |
| | GPAPQCIIPNKCTPPNVENGILVSDNRSLFSLNEV |
| | VEFRCQPGFVMKGPRRVKCQALNKWEPELPSCSRV |
| | CQPPPEILHGEHTPSHQDNESPGQEVFYSCEPGYD |
| | LRGAASLHCTPQGDWSPEAPRCAVKSCDDFLGQLP |
| | HGRVLFPLNLQLGAKVSFVCDEGFRLKGSSVSHCV |
| | LVGMRSLWNNSVPVCEHIFCPNPPAILNGRHTGTP |
| | SGDIPYGKEISYTCDPHPDRGMTFNLIGESTIRCT |
| | SDPHGNGVWSSPAPRCELSVRAGHCKTPEQFPFAS |
| | PTIPINDFEFPVGTSLNYECRPGYFGKMFSISCLE |
| | NLVWSSVEDNCRRKSCGPPPEPENGMVHINTDTQF |
| | GSTVNYSCNEGERLIGSPSTTCLVSGNNVTWDKKA |
| | PICEIISCEPPPTISNGDFYSNNRTSFHNGTVVTY |
| | QCHTGPDGEQLFELVGERSIYCTSKDDQVGVWSSP |
| | PPRCISTNKCTAPEVENAIRVPGNRSFFTLTEIIR |
| | FRCQPGFVMVGSHTVQCQTNGRWGPKLPHCSRVCQ |
| | PPPEILHGEHTLSHQDNESPGQEVFYSCEPSYDLR |
| | GAASLHCTPQGDWSPEAPRCTVKSCDDFLGQLPHG |
| | RVLLPLNLQLGAKVSFVCDEGFRLKGRSASHCVLA |
| | GMKALWNSSVPVCEQIFCPNPPAILNGRHTGTPFG |
| | DIPYGKEISYACDTHPDRGMTFNLIGESSIRCTSD |
| | PQGNGVWSSPAPRCELSVPAACPHPPKIQNGHYIG |
| | GHVSLYLPGMTISYICDPGYLLVGKGFIFCTDQGI |
| | WSQLDHYCKEVNCSFPLFMNGISKELEMKKVYHYG |
| | DYVTLKCEDGYTLEGSPWSQCQADDRWDPPLAKCT |
| | SRTHDALIVGTLSGTIFFILLIIFLSWIILKHRKG |
| | NNAHENPKEVAIHLHSQGGSSVHPRTLQTNEENSR |
| | VLP |

What is claimed is:

1. A modified mammalian cell in which a gene selected from the group consisting of SIGLEC14, MUC15, GPC4, TEX10, and combinations thereof is overexpressed.

2. The modified mammalian cell of claim 1, wherein at least two genes selected from the group consisting of SIGLEC14, MUC15, GPC4, and TEX10 are overexpressed.

3. The modified mammalian cell of claim 2, wherein at least three genes selected from the group consisting of SIGLEC14, MUC15, GPC4, and TEX10 are overexpressed.

4. The modified mammalian cell of claim 3, wherein SIGLEC14, MUC15, GPC4, and TEX10 are overexpressed.

5. The modified mammalian cell of any one of claims 1-4, further characterized by overexpression of:
(a) one or more genes associated with inhibition of macrophage phagocytosis;
(b) one or more genes associated with inhibition of complement activation and deposition;
(c) one or more genes associated with inhibition of natural killer (NK) cell activation; or
(d) any combination of (a), (b), and (c).

6. The mammalian cell of claim 5, wherein the one or more genes associated with inhibition of macrophage phagocytosis comprises CD47.

7. The mammalian cell of claim 5 or 6, wherein the one or more genes associated with complement activation and deposition comprise a gene selected from the group consisting of CD55, CD59, CD46, and combinations thereof.

8. The mammalian cell of any one of claims 5-7, wherein the one or more genes associated with inhibition of NK cell activation comprise a gene selected from the group consisting of HLA-G, HLA-E, and a combination thereof.

9. The mammalian cell of claim 5, characterized by:
(a) overexpression of one or more genes associated with inhibition of macrophage phagocytosis;
(b) overexpression of one or more genes associated with inhibition of complement activation and deposition; and
(c) overexpression of one or more genes associated with inhibition of NK cell activation.

10. The modified mammalian cell of claim 9, characterized by:
(a) overexpression of CD47;
(b) overexpression of CD55, CD59, and CD46; and
(c) overexpression of HLA-G and of HLA-E.

11. The modified mammalian cell of claim 5, characterized by:
(a) overexpression of one or more genes associated with inhibition of macrophage phagocytosis;
(b) overexpression of one or more genes associated with inhibition of complement activation and deposition; and
(c) normal expression of HLA-G and of HLA-E.

12. The modified mammalian cell of claim 11, characterized by:
(a) overexpression of CD47;
(b) overexpression of CD55, CD59, and CD46; and
(c) normal expression of HLA-G and of HLA-E.

13. The modified mammalian cell of claim 5, characterized by:
(a) normal expression of CD47;
(b) overexpression of one or more genes associated with inhibition of complement activation and deposition; and
(c) overexpression of one or more genes associated with inhibition of NK cell activation.

14. The modified mammalian cell of claim 13, characterized by:
(a) normal expression of CD47;
(b) overexpression of CD55, CD59, and CD46; and
(c) overexpression of HLA-G and of HLA-E.

15. The modified mammalian cell of claim 5, characterized by:
(a) overexpression of one or more genes associated with inhibition of macrophage phagocytosis;
(b) normal expression of CD55, CD59, and CD46; and
(c) overexpression of one or more genes associated with inhibition of NK cell activation.

16. The modified mammalian cell of claim 15, characterized by:
(a) overexpression of CD47;
(b) normal expression of CD55, CD59, and CD46; and
(c) overexpression of HLA-G and of HLA-E.

17. The modified mammalian cell of claim 5, characterized by:
(a) overexpression of one or more genes associated with inhibition of macrophage phagocytosis;
(b) normal expression of CD55, CD59, and CD46; and
(c) normal expression of HLA-G and of HLA-E.

18. The modified mammalian cell of claim 17, characterized by:
(a) overexpression of CD47;
(b) normal expression of CD55, CD59, and CD46; and
(c) normal expression of HLA-G and of HLA-E.

19. The modified mammalian cell of claim 5, characterized by:
(a) normal expression of CD47;
(b) overexpression of one or more genes associated with inhibition of complement activation and deposition; and
(c) normal expression of HLA-G and of HLA-E.

20. The modified mammalian cell of claim 19, characterized by:
(a) normal expression of CD47;
(b) overexpression of CD55, CD59, and CD46; and
(c) normal expression of HLA-G and of HLA-E.

21. The modified mammalian cell of any one of claims 1-20, wherein each overexpressed gene is overexpressed by at least 1.5-fold relative to expression in a control cell.

22. The modified mammalian cell of any one of claims 1-21, comprising one or more exogenous nucleic acid expression constructs, wherein each exogenous nucleic acid expression construct (1) encodes a gene and (2) comprises a promoter operably linked to the gene.

23. The method of claim 22, wherein the exogenous nucleic acid expression construct is a lentiviral construct.

24. The method of claim 22, wherein the exogenous nucleic acid expression construct is an adeno-associated virus (AAV) construct.

25. The modified mammalian cell of any one of claims 1-24, comprising one or more exogenous gene activation systems, wherein each exogenous gene activation system is capable of driving expression of an endogenous gene.

26. The modified mammalian cell of claim 25, wherein the gene activation system is a CRISPR activation system.

27. The mammalian cell of claim 26, wherein said CRISPR activation system comprises:
(a) an endonuclease-inactive Cas9 fused to a transcriptional activator; and
(b) a guide RNA (gRNA) that is capable of hybridizing to an endogenous gene.

28. The modified mammalian cell of any one of claims 1-27, further characterized by abrogated expression of one or more genes associated with T-cell activation.

29. The modified mammalian cell of claim 28, wherein the one or more genes associated with T-cell activation are selected from the group consisting of B2M, TAP1, CD74, CIITA, and combinations thereof.

30. The modified mammalian cell of any one of claims 1-29, further characterized by abrogated expression of one or more genes associated with NK cell activation.

31. The modified mammalian cell of claim 30, wherein the one or more genes associated with NK cell activation comprise one or more NKG2D ligands.

32. The modified mammalian cell of claim 31, wherein the one or more NKG2D ligands are selected from the group consisting of MICA, MICB, and a combination thereof.

33. The modified mammalian cell of any one of claims 1-32, characterized by abrogated expression of B2M, TAP1, CD74, CIITA, MICA, and MICB.

34. The modified mammalian cell of any one of claims 1-33, which is a human cell.

35. The modified mammalian cell of any one of claims 1-34, wherein the mammalian cell is a stem cell.

36. The modified mammalian stem cell of claim 35, wherein the stem cell is an induced pluripotent stem cell (iPSC).

37. The modified mammalian stem cell of claim 35, wherein the stem cell is an embryonic stem cell (ESC).

38. The modified mammalian cell of any one of claims 1-37, which is capable of evading immune rejection as determined by an in vitro assay.

39. The modified mammalian cell of any one of claims 1-38, which is capable of evading immune rejection as determined by an in vivo assay.

40. A cell or tissue that is differentiated from the modified mammalian cell of any one of claims 1-39.

41. A method comprising: administering the modified mammalian cell of any one of claims 1-39 or the cell or tissue of claim 40 to a mammalian subject.

42. The method of claim 41, wherein the mammalian subject is deficient in a cell type, and
(1) the method comprises administering a modified mammalian stem cell of any one of claims 1-39, and the modified mammalian stem cell is capable of developing into said cell type; or
(2) the method comprises administering the cell or tissue of claim 40, and
(i) the cell or tissue is capable of developing into said cell type or
(ii) the cell or tissue comprises cells of said cell type.

43. The method of claim 41 or 42, wherein the mammalian subject has been diagnosed with or is at risk for diabetes.

44. The method of any one of claims 41-43, wherein the mammalian subject is human.


SEQUENCE LISTING (40 sequences)

SEQ ID NO: 1 - DNA, 1086 bp, Homo sapiens
  source 1..1086: mol_type = genomic DNA
atgggcataatacaatctattcttgccacatcaagggattgttattcctttaaaaaaaaaccaataccaaagaagcctacaatgttggccttagccaaaattctgttgatttcaacgttgttttattcacttctatcggggagccatggaaaagaaaatcaagacataaacacaacacagaacattgcagaagtttttaaaacaatggaaaataaacctatttctttggaaagtgaagcaaacttaaactcagataaagaaaatataaccacctcaaatctcaaggcgagtcattcccctcctttgaatctacccaacaacagccacggaataacagatttctccagtaactcatcagcagagcattctttgggcagtctaaaacccacatctaccatttccacaagccctcccttgatccatagctttgtttctaaagtgccttggaatgcacctatagcagatgaagatcttttgcccatctcagcacatcccaatgctacacctgctctgtcttcagaaaacttcacttggtctttggtcaatgacaccgtgaaaactcctgataacagttccattacagttagcatcctctcttcagaaccaacttctccatctgtgacccccttgatagtggaaccaagtggatggcttaccacaaacagtgatagcttcactgggtttaccccttatcaagaaaaaacaactctacagcctaccttaaaattcaccaataattcaaaactctttccaaatacgtcagatccccaaaaagaaaatagaaatacaggaatagtattcggggccattttaggtgctattctgggtgtctcattgcttactcttgtgggctacttgttgtgtggaaaaaggaaaacggattcattttcccatcggcgactttatgacgacagaaatgaaccagttctgcgattagacaatgcaccggaaccttatgatgtgagttttgggaattctagctactacaatccaactttgaatgattcagccatgccagaaagtgaagaaaatgcacgtgatggcattcctatggatgacatacctccacttcgtacttctgtatag

SEQ ID NO: 2 - protein, 361 aa, Homo sapiens
MGIIQSILATSRDCYSFKKKPIPKKPTMLALAKILLISTLFYSLLSGSHGKENQDINTTQNIAEVFKTMENKPISLESEANLNSDKENITTSNLKASHSPPLNLPNNSHGITDFSSNSSAEHSLGSLKPTSTISTSPPLIHSFVSKVPWNAPIADEDLLPISAHPNATPALSSENFTWSLVNDTVKTPDNSSITVSILSSEPTSPSVTPLIVEPSGWLTTNSDSFTGFTPYQEKTTLQPTLKFTNNSKLFPNTSDPQKENRNTGIVFGAILGAILGVSLLTLVGYLLCGKRKTDSFSHRRLYDDRNEPVLRLDNAPEPYDVSFGNSSYYNPTLNDSAMPESEENARDGIPMDDIPPLRTSV

SEQ ID NO: 3 - DNA, 936 bp, Homo sapiens
  source 1..936: mol_type = genomic DNA
atgggcataatacaatctattcttgccacatcaagggattgttattcctttaaaaaaaaaccaataccaaagaagcctacaatgttggccttagccaaaattctgttgatttcaacgttgttttattcacttctatcggggagccatggaaaagaaaatcaagacataaacacaacacagaacattgcagaagtttttaaaacaatggaaaataaacctatttctttggaaagtgaagcaaacttaaactcagataaagaaaatataaccacctcaaatctcaaggcgagtcattcccctcctttgaatctacccaacaacagccacggaataacagatttctccagtaactcatcagcagagcattctttgggcagtctaaaacccacatctaccatttccacaagccctcccttgatccatagctttgtttctaaagtgccttggaatgcacctatagcagatgaagatcttttgcccatctcagcacatcccaatgctacacctgctctgtcttcagaaaacttcacttggtctttggtcaatgacaccgtgaaaactcctgataacagttccattacagttagcatcctctcttcagaaccaacttctccatctgtgacccccttgatagtggaaccaagtggatggcttaccacaaacagtgatagcttcactgggtttaccccttatcaagaaaaaacaactctacagcctaccttaaaattcaccaataattcaaaactctttccaaatacgtcagatccccaaaaagttctgcgattagacaatgcaccggaaccttatgatgtgagttttgggaattctagctactacaatccaactttgaatgattcagccatgccagaaagtgaagaaaatgcacgtgatggcattcctatggatgacatacctccacttcgtacttctgtatag

SEQ ID NO: 4 - protein, 311 aa, Homo sapiens
MGIIQSILATSRDCYSFKKKPIPKKPTMLALAKILLISTLFYSLLSGSHGKENQDINTTQNIAEVFKTMENKPISLESEANLNSDKENITTSNLKASHSPPLNLPNNSHGITDFSSNSSAEHSLGSLKPTSTISTSPPLIHSFVSKVPWNAPIADEDLLPISAHPNATPALSSENFTWSLVNDTVKTPDNSSITVSILSSEPTSPSVTPLIVEPSGWLTTNSDSFTGFTPYQEKTTLQPTLKFTNNSKLFPNTSDPQKVLRLDNAPEPYDVSFGNSSYYNPTLNDSAMPESEENARDGIPMDDIPPLRTSV

SEQ ID NO: 5 - DNA, 1005 bp, Homo sapiens
  source 1..1005: mol_type = genomic DNA
atgttggccttagccaaaattctgttgatttcaacgttgttttattcacttctatcggggagccatggaaaagaaaatcaagacataaacacaacacagaacattgcagaagtttttaaaacaatggaaaataaacctatttctttggaaagtgaagcaaacttaaactcagataaagaaaatataaccacctcaaatctcaaggcgagtcattcccctcctttgaatctacccaacaacagccacggaataacagatttctccagtaactcatcagcagagcattctttgggcagtctaaaacccacatctaccatttccacaagccctcccttgatccatagctttgtttctaaagtgccttggaatgcacctatagcagatgaagatcttttgcccatctcagcacatcccaatgctacacctgctctgtcttcagaaaacttcacttggtctttggtcaatgacaccgtgaaaactcctgataacagttccattacagttagcatcctctcttcagaaccaacttctccatctgtgacccccttgatagtggaaccaagtggatggcttaccacaaacagtgatagcttcactgggtttaccccttatcaagaaaaaacaactctacagcctaccttaaaattcaccaataattcaaaactctttccaaatacgtcagatccccaaaaagaaaatagaaatacaggaatagtattcggggccattttaggtgctattctgggtgtctcattgcttactcttgtgggctacttgttgtgtggaaaaaggaaaacggattcattttcccatcggcgactttatgacgacagaaatgaaccagttctgcgattagacaatgcaccggaaccttatgatgtgagttttgggaattctagctactacaatccaactttgaatgattcagccatgccagaaagtgaagaaaatgcacgtgatggcattcctatggatgacatacctccacttcgtacttctgtatag

SEQ ID NO: 6 - protein, 334 aa, Homo sapiens
MLALAKILLISTLFYSLLSGSHGKENQDINTTQNIAEVFKTMENKPISLESEANLNSDKENITTSNLKASHSPPLNLPNNSHGITDFSSNSSAEHSLGSLKPTSTISTSPPLIHSFVSKVPWNAPIADEDLLPISAHPNATPALSSENFTWSLVNDTVKTPDNSSITVSILSSEPTSPSVTPLIVEPSGWLTTNSDSFTGFTPYQEKTTLQPTLKFTNNSKLFPNTSDPQKENRNTGIVFGAILGAILGVSLLTLVGYLLCGKRKTDSFSHRRLYDDRNEPVLRLDNAPEPYDVSFGNSSYYNPTLNDSAMPESEENARDGIPMDDIPPLRTSV

SEQ ID NO: 7 - protein, 311 aa, Homo sapiens
KENQDINTTQNIAEVFKTMENKPISLESEANLNSDKENITTSNLKASHSPPLNLPNNSHGITDFSSNSSAEHSLGSLKPTSTISTSPPLIHSFVSKVPWNAPIADEDLLPISAHPNATPALSSENFTWSLVNDTVKTPDNSSITVSILSSEPTSPSVTPLIVEPSGWLTTNSDSFTGFTPYQEKTTLQPTLKFTNNSKLFPNTSDPQKENRNTGIVFGAILGAILGVSLLTLVGYLLCGKRKTDSFSHRRLYDDRNEPVLRLDNAPEPYDVSFGNSSYYNPTLNDSAMPESEENARDGIPMDDIPPLRTSV

SEQ ID NO: 8 - DNA, 1191 bp, Homo sapiens
  source 1..1191: mol_type = genomic DNA
atgctgcccctgctgctgctgcccctgctgtggggggggtccctgcaggagaagccagtgtacgagctgcaagtgcagaagtcggtgacggtgcaggagggcctgtgcgtccttgtgccctgctccttctcttacccctggagatcctggtattcctctcccccactctacgtctactggttccgggacggggagatcccatactacgctgaggttgtggccacaaacaacccagacagaagagtgaagccagagacccagggccgattccgcctccttggggatgtccagaagaagaactgctccctgagcatcggagatgccagaatggaggacacgggaagctatttcttccgcgtggagagaggaagggatgtaaaatatagctaccaacagaataagctgaacttggaggtgacagccctgatagagaaacccgacatccactttctggagcctctggagtccggccgccccacaaggctgagctgcagccttccaggatcctgtgaagcgggaccacctctcacattctcctggacggggaatgccctcagccccctggaccccgagaccacccgctcctcggagctcaccctcacccccaggcccgaggaccatggcaccaacctcacctgtcaggtgaaacgccaaggagctcaggtgaccacggagagaactgtccagctcaatgtctcctatgctccacagaacctcgccatcagcatcttcttcagaaatggcacaggcacagccctgcggatcctgagcaatggcatgtcggtgcccatccaggagggccagtccctgttcctcgcctgcacagttgacagcaacccccctgcctcactgagctggttccgggagggaaaagccctcaatccttcccagacctcaatgtctgggaccctggagctgcctaacataggagctagagagggaggggaattcacctgccgggttcagcatccgctgggctcccagcacctgtccttcatcctttctgtgcagagaagctcctcttcctgcatatgtgtaactgagaaacagcagggctcctggcccctcgtcctcaccctgatcaggggggctctcatgggggctggcttcctcctcacctatggcctcacctggatctactataccaggtgtggaggcccccagcagagcagggctgagaggcctggctga

SEQ ID NO: 9 - protein, 396 aa, Homo sapiens
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLTLIRGALMGAGFLLTYGLTWIYYTRCGGPQQSRAERPG

SEQ ID NO: 10 - protein, 380 aa, Homo sapiens
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLTLIRGALMGAGFLLTYGLTWIYYTRCGGPQQSRAERPG

SEQ ID NO: 11 - DNA, 2790 bp, Homo sapiens
  source 1..2790: mol_type = genomic DNA
atgactaaaaaaagaaaacgccaacatgattttcaaaaagtaaaattgaaagttggtaaaaagaagcccaagttacaaaatgctactcctacaaactttaaaacaaagactatacatctgcctgagcaactcaaagaggatggaacacttccaacaaacaatagaaaacttaacataaaggatttgctgtcacagatgcatcactacaatgctggggttaaacaaagtgctcttcttggacttaaagaccttttgtctcaatacccatttataattgatgcacacctttcaaacatattaagtgaagtgactgctgtgtttacagataaagatgctaatgtacgattagcagcagttcaacttcttcaattcctggcccccaaaatacgagctgaacaaatttctccattttttcctttggtaagtgcccatctctctagtgccatgactcacattactgaaggaattcaggaggactctttaaaagttttggacattctgctggaacagtacccagctctaattactggccgtagcagcatattgcttaagaattttgtagaacttatttctcatcagcagctgtccaaaggactgataaatagagacagatcccagtcctggatactttctgtaaatcctaatcggagactcacttctcagcaatggaggctgaaagtcttagtgagactcagtaaattccttcaggccttggcagatggatccagtaggttgagagaaagtgaaggacttcaggaacagaaagaaaatccccatgccactagcaactccatttttatcaactggaaggaacatgccaacgaccagcaacacatccaggtttatgaaaatgggggttcacagccaaatgtcagttcacagttcaggctacggtatctggttggaggactgagtggtgtggatgaaggcctgtcatctactgaaaacctgaaaggatttattgagataataattccattgctaattgaatgctgggttgaagctgtacctccacaactagctactcctgttgggaatggtatagaacgagaacctctacaggttatgcagcaagttcttaatattatttcccttctgtggaaactctctaaacaacaggatgaaacccataaattggagtcatggcttcgaaagaactaccttattgattttaaacaccattttatgagtcgttttccatatgtcttaaaagaaataaccaagcacaaaaggaaagagccaaataaaagcatcaagcattgcacagttctctccaataacatagatcatctcttactgaatttaacactgtctgatatcatggtctccctggcaaatgcgtcaactttgcagaaggattgcagttggatagaaatgataaggaaatttgtaacagagacccttgaagatggctctaggctaaatagtaagcaactgaacagattgctgggagtatcctggaggttaatgcaaatacagccaaacagagaggacacagagactcttattaaggcagtttatacattatatcagcagaggggccttatccttccagttcggactttgttattgaagtttttcagtaaaatctatcagacagaagaactgagatcttgtagattcagatatcgtagtaaagtgttatcccgttggctggctggcttaccattgcaacttgctcatcttggctcccgaaatcctgagctctctacacagcttatcgatatcattcataccgctgcagcacgagcaaataaagaattactaaaaagtttacaagctactgccctccgaatttatgatccacaagaaggtgctgtggtggttctccctgcagactctcagcagcgtttggttcagcttgtatatttcctacccagtctgccggctgatttgctttctcggttaagtcgttgctgtattatgggaagactcagttcaagtttggctgccatgcttatcgggatactgcacatgagatcatcattttctgggtggaagtattcagctaaagactggttgatgagtgatgtagactatttcagcttcttattttccacacttacagggttttcgaaagaggagttgacttggcttcagagccttcgaggagttcctcatgtcatccagacacagctttcccctgtgcttctctaccttacagatttggatcaatttttacaccactgggatgtaacagaggcagtttttcacagtttattggttattcctgcccgaagtcagaactttgacatcttgcaaagtgccatcagtaagcatttggttgggttgactgtaattcctgacagcacggctggctgtgtttttggtgttatctgtaagctcctggatcatacttgtgtagttagtgagactctactgccatttctggcttcttgttgctacagtcttctttattttctgctcactatagagaaaggggaagcagaacatctaagaaagagggacaagctgtggggggtctgtgtctccatcctggctctcttgcctcgagtcctcaggttgatgctgcagagcctgcgggtgaacagagttgggcctgaggagctgcctgttgtgggccagctgcttcgactgctgcttcagcatgcacccctcaggactcatatgttgaccaatgcgatcttggtgcagcagatcatcaagaatatcacgacattgaagagtggaagtgttcaggaacagtggctcacagacttacattactgcttcaacgtgtatatcactgggcatccccaagggcccagtgcactggctacagtgtattga

SEQ ID NO: 12 - protein, 929 aa, Homo sapiens
MTKKRKRQHDFQKVKLKVGKKKPKLQNATPTNFKTKTIHLPEQLKEDGTLPTNNRKLNIKDLLSQMHHYNAGVKQSALLGLKDLLSQYPFIIDAHLSNILSEVTAVFTDKDANVRLAAVQLLQFLAPKIRAEQISPFFPLVSAHLSSAMTHITEGIQEDSLKVLDILLEQYPALITGRSSILLKNFVELISHQQLSKGLINRDRSQSWILSVNPNRRLTSQQWRLKVLVRLSKFLQALADGSSRLRESEGLQEQKENPHATSNSIFINWKEHANDQQHIQVYENGGSQPNVSSQFRLRYLVGGLSGVDEGLSSTENLKGFIEIIIPLLIECWVEAVPPQLATPVGNGIEREPLQVMQQVLNIISLLWKLSKQQDETHKLESWLRKNYLIDFKHHFMSRFPYVLKEITKHKRKEPNKSIKHCTVLSNNIDHLLLNLTLSDIMVSLANASTLQKDCSWIEMIRKFVTETLEDGSRLNSKQLNRLLGVSWRLMQIQPNREDTETLIKAVYTLYQQRGLILPVRTLLLKFFSKIYQTEELRSCRFRYRSKVLSRWLAGLPLQLAHLGSRNPELSTQLIDIIHTAAARANKELLKSLQATALRIYDPQEGAVVVLPADSQQRLVQLVYFLPSLPADLLSRLSRCCIMGRLSSSLAAMLIGILHMRSSFSGWKYSAKDWLMSDVDYFSFLFSTLTGFSKEELTWLQSLRGVPHVIQTQLSPVLLYLTDLDQFLHHWDVTEAVFHSLLVIPARSQNFDILQSAISKHLVGLTVIPDSTAGCVFGVICKLLDHTCVVSETLLPFLASCCYSLLYFLLTIEKGEAEHLRKRDKLWGVCVSILALLPRVLRLMLQSLRVNRVGPEELPVVGQLLRLLLQHAPLRTHMLTNAILVQQIIKNITTLKSGSVQEQWLTDLHYCFNVYITGHPQGPSALATVY

SEQ ID NO: 13 - DNA, 2742 bp, Homo sapiens
  source 1..2742: mol_type = genomic DNA
atgtcgagaatgactaaaaaaagaaaacgccaacatgattttcaaaaagtaaaattgaaagttggtaaaaagaagcccaagttacaaaatgctactcctacaaactttaaaacaaagactatacatctgcctgagcaactcaaagaggatggaacacttccaacaaacaatagaaaacttaacataaaggatttgctgtcacagatgcatcactacaatgctggggttaaacaaagtgctcttcttggacttaaagaccttttgtctcaatacccatttataattgatgcacacctttcaaacatattaagtgaagtgactgctgtgtttacagataaagatgctaatgtacgattagcagcagttcaacttcttcaattcctggcccccaaaatacgagctgaacaaatttctccattttttcctttggtaagtgcccatctctctagtgccatgactcacattactgaaggaattcaggaggactctttaaaagttttggacattctgctggaacagtacccagctctaattactggccgtagcagcatattgcttaagaattttgtagaacttatttctcatcagcagctgtccaaaggactgataaatagagacagatcccagtcctggatactttctgtaaatcctaatcggagactcacttctcagcaatggaggctgaaagtcttagtgagactcagtaaattccttcaggccttggcagatggatccagtaggttgagagaaagtgaaggacttcaggaacagaaagaaaatccccatgccactagcaactccatttttatcaactggaaggaacatgccaacgaccagcaacacatccaggtttatgaaaatgggggttcacagccaaatgtcagttcacagttcaggctacggtatctggttggaggactgagtggtgtggatgaaggcctgtcatctactgaaaacctgaaaggatttattgagataataattccattgctaattgaatgctgggttgaagctgtacctccacaactagctactcctgttgggaatggtatagaacgagaacctctacaggttatgcagcaagttcttaatattatttcccttctgtggaaactctctaaacaacaggatgaaacccataaattggagtcatggcttcgaaagaactaccttattgattttaaacaccattttatgagtcgttttccatatgtcttaaaagaaataaccaagcacaaaaggaaagagccaaataaaagcatcaagcattgcacagttctctccaataacatagatcatctcttactgaatttaacactgtctgatatcatggtctccctggcaaatgcgtcaactttgcagaaggattgcagttggatagaaatgataaggaaatttgtaacagagacccttgaagatggctctaggctaaatagtaagcaactgaacagattgctgggagtatcctggaggttaatgcaaatacagccaaacagagaggacacagagactcttattaaggcagtttatacattatatcagcagaggggccttatccttccagttcggactttgttattgaagtttttcagtaaaatctatcagacagaagaactgagatcttgtagattcagatatcgtagtaaagtgttatcccgttggctggctggcttaccattgcaacttgctcatcttggctcccgaaatcctgagctctctacacagcttatcgatatcattcataccgctgcagcacgagcaaataaagaattactaaaaagtttacaagctactgccctccgaatttatgatccacaagaaggtgctgtggtggttctccctgcagactctcagcagcgtttggttcagcttgtatatttcctacccagtctgccggctgatttgctttctcggttaagtcgttgctgtattatgggaagactcagttcaagtttggctgccatgcttatcgggatactgcacatgagatcatcattttctgggtggaagtattcagctaaagactggttgatgagtgatgtagactatttcagcttcttattttccacacttacagggttttcgaaagaggagttgacttggcttcagagccttcgaggagttcctcatgtcatccagacacagctttcccctgtgcttctctaccttacagatttggatcaatttttacaccactgggatgtaacagaggcagtttttcacagtttattggttattcctgcccgaagtcagaactttgacatcttgcaaagtgccatcagtaagcatttggttgggttgactgtaattcctgacagcacggctggctgtgtttttggtgttatctgtaagctcctggatcatacttgtgtagttagtgagactctactgccatttctggcttcttgttgctacagtcttctttattttctgctcactatagagaaaggggaagcagaacatctaagaaagaggttgatgctgcagagcctgcgggtgaacagagttgggcctgaggagctgcctgttgtgggccagctgcttcgactgctgcttcagcatgcacccctcaggactcatatgttgaccaatgcgatcttggtgcagcagatcatcaagaatatcacgacattgaagagtggaagtgttcaggaacagtggctcacagacttacattactgcttcaacgtgtatatcactgggcatccccaagggcccagtgcactggctacagtgtattga

SEQ ID NO: 14 - protein, 913 aa, Homo sapiens
MSRMTKKRKRQHDFQKVKLKVGKKKPKLQNATPTNFKTKTIHLPEQLKEDGTLPTNNRKLNIKDLLSQMHHYNAGVKQSALLGLKDLLSQYPFIIDAHLSNILSEVTAVFTDKDANVRLAAVQLLQFLAPKIRAEQISPFFPLVSAHLSSAMTHITEGIQEDSLKVLDILLEQYPALITGRSSILLKNFVELISHQQLSKGLINRDRSQSWILSVNPNRRLTSQQWRLKVLVRLSKFLQALADGSSRLRESEGLQEQKENPHATSNSIFINWKEHANDQQHIQVYENGGSQPNVSSQFRLRYLVGGLSGVDEGLSSTENLKGFIEIIIPLLIECWVEAVPPQLATPVGNGIEREPLQVMQQVLNIISLLWKLSKQQDETHKLESWLRKNYLIDFKHHFMSRFPYVLKEITKHKRKEPNKSIKHCTVLSNNIDHLLLNLTLSDIMVSLANASTLQKDCSWIEMIRKFVTETLEDGSRLNSKQLNRLLGVSWRLMQIQPNREDTETLIKAVYTLYQQRGLILPVRTLLLKFFSKIYQTEELRSCRFRYRSKVLSRWLAGLPLQLAHLGSRNPELSTQLIDIIHTAAARANKELLKSLQATALRIYDPQEGAVVVLPADSQQRLVQLVYFLPSLPADLLSRLSRCCIMGRLSSSLAAMLIGILHMRSSFSGWKYSAKDWLMSDVDYFSFLFSTLTGFSKEELTWLQSLRGVPHVIQTQLSPVLLYLTDLDQFLHHWDVTEAVFHSLLVIPARSQNFDILQSAISKHLVGLTVIPDSTAGCVFGVICKLLDHTCVVSETLLPFLASCCYSLLYFLLTIEKGEAEHLRKRLMLQSLRVNRVGPEELPVVGQLLRLLLQHAPLRTHMLTNAILVQQIIKNITTLKSGSVQEQWLTDLHYCFNVYITGHPQGPSALATVY

SEQ ID NO: 15 - DNA, 1671 bp, Homo sapiens
  source 1..1671: mol_type = genomic DNA
atggcacggttcggcttgcccgcgcttctctgcaccctggcagtgctcagcgccgcgctgctggctgccgagctcaagtcgaaaagttgctcggaagtgcgacgtctttacgtgtccaaaggcttcaacaagaacgatgcccccctccacgagatcaacggtgatcatttgaagatctgtccccagggttctacctgctgctctcaagagatggaggagaagtacagcctgcaaagtaaagatgatttcaaaagtgtggtcagcgaacagtgcaatcatttgcaagctgtctttgcttcacgttacaagaagtttgatgaattcttcaaagaactacttgaaaatgcagagaaatccctgaatgatatgtttgtgaagacatatggccatttatacatgcaaaattctgagctatttaaagatctcttcgtagagttgaaacgttactacgtggtgggaaatgtgaacctggaagaaatgctaaatgacttctgggctcgcctcctggagcggatgttccgcctggtgaactcccagtaccactttacagatgagtatctggaatgtgtgagcaagtatacggagcagctgaagcccttcggagatgtccctcgcaaattgaagctccaggttactcgtgcttttgtagcagcccgtactttcgctcaaggcttagcggttgcgggagatgtcgtgagcaaggtctccgtggtaaaccccacagcccagtgtacccatgccctgttgaagatgatctactgctcccactgccggggtctcgtgactgtgaagccatgttacaactactgctcaaacatcatgagaggctgtttggccaaccaaggggatctcgattttgaatggaacaatttcatagatgctatgctgatggtggcagagaggctagagggtcctttcaacattgaatcggtcatggatcccatcgatgtgaagatttctgatgctattatgaacatgcaggataatagtgttcaagtgtctcagaaggttttccagggatgtggaccccccaagcccctcccagctggacgaatttctcgttccatctctgaaagtgccttcagtgctcgcttcagaccacatcaccccgaggaacgcccaaccacagcagctggcactagtttggaccgactggttactgatgtcaaggagaaactgaaacaggccaagaaattctggtcctcccttccgagcaacgtttgcaacgatgagaggatggctgcaggaaacggcaatgaggatgactgttggaatgggaaaggcaaaagcaggtacctgtttgcagtgacaggaaatggattagccaaccagggcaacaacccagaggtccaggttgacaccagcaaaccagacatactgatccttcgtcaaatcatggctcttcgagtgatgaccagcaagatgaagaatgcatacaatgggaacgacgtggacttctttgatatcagtgatgaaagtagtggagaaggaagtggaagtggctgtgagtatcagcagtgcccttcagagtttgactacaatgccactgaccatgctgggaagagtgccaatgagaaagccgacagtgctggtgtccgtcctggggcacaggcctacctcctcactgtcttctgcatcttgttcctggttatgcagagagagtggagataa

SEQ ID NO: 16 - protein, 556 aa, Homo sapiens
MARFGLPALLCTLAVLSAALLAAELKSKSCSEVRRLYVSKGFNKNDAPLHEINGDHLKICPQGSTCCSQEMEEKYSLQSKDDFKSVVSEQCNHLQAVFASRYKKFDEFFKELLENAEKSLNDMFVKTYGHLYMQNSELFKDLFVELKRYYVVGNVNLEEMLNDFWARLLERMFRLVNSQYHFTDEYLECVSKYTEQLKPFGDVPRKLKLQVTRAFVAARTFAQGLAVAGDVVSKVSVVNPTAQCTHALLKMIYCSHCRGLVTVKPCYNYCSNIMRGCLANQGDLDFEWNNFIDAMLMVAERLEGPFNIESVMDPIDVKISDAIMNMQDNSVQVSQKVFQGCGPPKPLPAGRISRSISESAFSARFRPHHPEERPTTAAGTSLDRLVTDVKEKLKQAKKFWSSLPSNVCNDERMAAGNGNEDDCWNGKGKSRYLFAVTGNGLANQGNNPEVQVDTSKPDILILRQIMALRVMTSKMKNAYNGNDVDFFDISDESSGEGSGSGCEYQQCPSEFDYNATDHAGKSANEKADSAGVRPGAQAYLLTVFCILFLVMQREWR

SEQ ID NO: 17 - protein, 511 aa, Homo sapiens
ALLAAELKSKSCSEVRRLYVSKGFNKNDAPLHEINGDHLKICPQGSTCCSQEMEEKYSLQSKDDFKSVVSEQCNHLQAVFASRYKKFDEFFKELLENAEKSLNDMFVKTYGHLYMQNSELFKDLFVELKRYYVVGNVNLEEMLNDFWARLLERMFRLVNSQYHFTDEYLECVSKYTEQLKPFGDVPRKLKLQVTRAFVAARTFAQGLAVAGDVVSKVSVVNPTAQCTHALLKMIYCSHCRGLVTVKPCYNYCSNIMRGCLANQGDLDFEWNNFIDAMLMVAERLEGPFNIESVMDPIDVKISDAIMNMQDNSVQVSQKVFQGCGPPKPLPAGRISRSISESAFSARFRPHHPEERPTTAAGTSLDRLVTDVKEKLKQAKKFWSSLPSNVCNDERMAAGNGNEDDCWNGKGKSRYLFAVTGNGLANQGNNPEVQVDTSKPDILILRQIMALRVMTSKMKNAYNGNDVDFFDISDESSGEGSGSGCEYQQCPSEFDYNATDHAGKSANEKADS

SEQ ID NO: 18 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 19 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 20 - protein, 312 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 21 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 22 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 23 - protein, 287 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 24 - protein, 294 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 25 - protein, 275 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 26 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 27 - protein, 33 aa, Homo sapiens
SETTTGTSSNSSQSTSNSGLAPNPTNATTKAAG

SEQ ID NO: 28 - protein, 129 aa, Homo sapiens
MVGRFCPESPPGFVRVAATSAVSLDPPSGEPRPGCGYPGPRSAASRVYGCTAPARETGGWAWETLAGAGAKKIYSSETTTGTSSNSSQSTSNSGLAPNPTNATTKAAGGALQSTASLFVVSLSLLHLYS

SEQ ID NO: 29 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 30 - protein, 319 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 31 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 32 - protein, 77 aa, Homo sapiens
LQCYNCPNPTADCKTAVNCSSDFDACLITKAGLQVYNKCWKFEHCNFNDVTTRLRENELTYYCCKKDLCNFNEQLEN

SEQ ID NO: 33 - protein, 392 aa, Homo sapiens [sequence omitted]

SEQ ID NO: 34 - protein, 358 aa, Homo sapiens
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 35 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 36 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 37 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 38 - protein, 337 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 39 - protein, 2039 aa, Homo sapiens
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 40 - protein, 1998 aa, Homo sapiens
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP